(19) United States
(12) Patent Application Publication
(10) Pub. No.: US 2015/0247190 A1
(43) Pub. Date: Sep. 3, 2015

(54) METHODS AND SYSTEMS FOR MICROFLUIDICS IMAGING AND ANALYSIS

(71) Applicant: CALIFORNIA INSTITUTE OF TECHNOLOGY, Pasadena, CA (US)

(72) Inventors: Rustem F. Ismagilov, Altadena, CA (US); David A. Selck, Albambra, CA (US); Mikhail Karymov, San Gabriel, CA (US); Bing Sun, Qingdao (CN)

(21) Appl. No.: 14/433,602

(22) PCT Filed: Oct. 4, 2013
(86) PCT No.: PCT/US13/63594

§ 371 Date: Apr. 3, 2015

Related U.S. Application Data
(60) Provisional application No. 61/710,454, filed on Oct. 5, 2012.

(51) Int. Cl. C12Q 1/68; G01N 21/64; C12Q 1/70

(52) U.S. Cl. CPC C12Q 1/6851; C12Q 1/703; G01N 21/6428; G01N 21/6456; C12Q 2600/16; G01N 2021/6439; G01N 2021/6471; G01N 2201/061; G01N 2201/062; G01N 2201/12; G06F 19/16

(57) ABSTRACT

Disclosed herein are methods and devices for assessing sample for the presence of a disease or organism using images from devices such as a consumer cell phones.

### CROSS-REFERENCE

This application claims the benefit of U.S. Provisional Application No. 61/710,454, filed Oct. 5, 2012, which application is incorporated herein by reference in its entirety.

### STATEMENT AS TO FEDERALLY SPONSORED RESEARCH

This invention was made with government support under DARPA Cooperative Agreement HR0011-11-2-0006, NIH Grant R01EB012946 awarded by the National Institute of Biomedical Imaging and Bioengineering, and by the NIH Director's Pioneer Award program, part of the NIH Roadmap for Medical Research (5DP1OD003584). The government has certain rights in the invention.

### BACKGROUND OF THE INVENTION

Currently, many important quantitative diagnostic/detection tools are available only in complex laboratory settings. In the laboratory, two methods that are commonly used to quantify molecules: kinetic analysis and single-molecule counting. Kinetic analysis is the most common method, and includes tests such as real-time polymerase chain reaction (rt-PCR), in which the fluorescence readout of a PCR reaction is measured as a function of the cycle number and the acquired curves are compared to known concentrations to determine the specific sample concentration. While good results can be obtained with this type of analysis, complex and expensive laboratory equipment must be used in highly controlled environments.

With the development of consumer electronics, such as cell phones, it has become possible to use such devices as diagnostic/detection platforms. These devices are especially attractive in limited-resource settings, where there are limitations on trained personnel, infrastructure, medical instruments, and access to resources such as electricity and refrigeration. With the development of wireless telecommunication infrastructure and cloud-based technology, mobile communication devices could be used for imaging, processing, and communicating diagnostic/detection data in remote settings.

Several challenges exist for using consumer electronics for diagnostics or detection. Currently, many cell phone assays are based on analysis of lateral flow immune-chromatographic data. These tests can suffer from lack of accuracy and reliability due to analog ratiometric nature of the results. Variability between devices also creates challenges for using consumer electronics for diagnostic or detection purposes. Each user's phone may have different hardware and/or software which creates challenges for reliability and repeatability. Additionally, as the devices are used outside the controlled environment of the laboratory environmental differences such as changes in humidity or temperature can alter the ability of a consumer electronic to used with accuracy and precision.

### SUMMARY OF THE INVENTION

In one aspect, the invention provides a method for generating sample data comprising: i) emitting a set of photons from a light source in a short burst, the burst lasting from about 5/1,000,000 of a second to about one second, wherein at least a portion of the photons contact the sample; ii) collecting at least one photon with an image sensor to create sample data, wherein the collected photon had contacted the sample; iii) processing the sample data to create a binary quantification of nucleic acids in the sample; iv) analyzing the binary quantification of nucleic acids to generate a conclusion description relating to the sample.

In some embodiments, the quantification of nucleic acids in the sample is used to detect a non-nucleic acid component of the sample. In some embodiments, the non-nucleic acid component is selected from the group comprising cells, proteins and viruses.

In some embodiments, the collected photon was one of the photons emitted from the light source in a short burst.

In some embodiments, the photons comprise photons in the visible spectrum.

In some embodiments, the photons comprise photons in the UV spectrum.

In some embodiments, the light source is a camera flash or flash bulb.

In some embodiments, the light source is a Xenon flash.

In some embodiments, the light source is a light emitting diode (LED).

In some embodiments, the image sensor is a CMOS.

In some embodiments, the image sensor is a CCD.

In some embodiments, the intensity of the set of photons emitted is not constant during the length of time of the short burst.

In some embodiments, the data associated with the sample is an image or set of images that capture(s) a change in optical properties of the sample relative to a previous time point or a standard sample.

In some embodiments, the data associated with the sample is an image or set of images that capture(s) the presence or absence of fluorescence data. In some embodiments, the fluorescence data is the result of photons emitted from a fluorescent dye. In some embodiments, the fluorescent dye is SYTO9. In some embodiments, the fluorescent dye is calcein.

In some embodiments, the data associated with the sample is an image or set of images that capture(s) the presence or absence of colorimetric data.

In some embodiments, the data associated with the sample is an image or set of images that capture(s) the presence or absence of translucence data.

In some embodiments, the data associated with the sample is an image or set of images that capture(s) the presence or absence of translucence versus color data.

In some embodiments, the data associated with the sample is an image or set of images that capture(s) the presence or absence of opacity data.

In some embodiments, the data associated with the sample is single image captured completely simultaneously.

In some embodiments, the data associated with the sample comprises measurements from greater than one spatially-isolated compartment each of the compartments comprising a portion of the sample.

In some embodiments, processing the data further comprises utilizing size discrimination, shape discrimination, comparison to a standard or set of standards, or comparison by color within a single image to create a digital quantification of nucleic acids in the sample.

In some embodiments, processing the data further comprises: i) examining the data associated with sample and measuring for each at least one of the following characteristic thresholds a-e: a) at least one alignment feature is present and/or in the correct orientation; b) the data associated with the sample comprises an image in focus; c) the data associated with the image ensure proper usage of assay; d) the image comprises a graphical depiction of the intended sample; e) the dimensions of the sample match the intended dimensions; and f) the sample was distributed in a single container over a series of containers as intended; and ii) if one or more of the characteristic thresholds was not met, then adjusting the parameters required to exceed all characteristic thresholds and repeating all steps of the method described herein until an unmet characteristic thresholds is met.

In some embodiments, the data processing is done with a local computer.

In some embodiments, the data processing is done by transferring the data to a different device to be processed.

In some embodiments, at least one of the emitted photons that contacted the sample is of a shifted wavelength due to fluorescence.

In some embodiments, the conclusion description is a description of disease. In some embodiments, the conclusion description describes the presence or absence of genetic disorder. In some embodiments, the conclusion description is a quantification of a viral load. In some embodiments, the conclusion description is a diagnosis of a presence or absence of a viral infection. In some embodiments, the conclusion description is a quantification of at least one species of bacterium. In some embodiments, the conclusion description is a diagnosis of a presence or absence of a bacterial infection. In some embodiments, the conclusion description is the quantification of a gene in the sample.

In some embodiments of the method, the conclusion description is determining the presence or absence of a gene or nucleic acid sequence in the sample. In some embodiments, conclusion description is determining the presence or absence of a gene in the sample.

In some embodiments, conclusion description is determining the presence or absence of a DNA or RNA sequence in the sample. In some embodiments, conclusion description is determining the presence or absence of a mutation in a gene or a mutation in a nucleic acid sequence in the sample.

In some embodiments, conclusion description is the quantification of a mutation in a gene or nucleic acid sequence in the sample. In some embodiments of the methods described herein, the gene or nucleic acid sequence is plant derived. In some embodiments, the gene or nucleic acid sequence is human derived. In some embodiments, wherein the gene or nucleic acid sequence is virus derived. In some embodiments, wherein the gene or nucleic acid sequence is bacterium derived.

In some embodiments, the method further comprises displaying and/or associating in non-transitory computer readable media database the conclusion description and other information.

In some embodiments, the other information is information about an organism from which the sample was collected. In some embodiments, the other information comprises patient name, age, weight, height, time of sample collection, type of sample, GPS location data pertaining to sample collection and/or data collection, or medical records.

In some embodiments, the method further comprises displaying the conclusion description. In some embodiments, the conclusion description is displayed to the user. In some embodiments, the conclusion description is sent to a different device.

In some embodiments, the sample comprises at least one nucleic acid. In some embodiments, the nucleic acid is obtained from a human.

In some embodiments, the nucleic acid is obtained from a plant or plant seed. In some embodiments, the nucleic acid is obtained from an animal. In some embodiments, the nucleic acid is obtained from a bacterium. In some embodiments, the nucleic acid is obtained from a virus. In some embodiments, the nucleic acid is synthetic. In some embodiments, the nucleic acid is derived from an unknown source.

In some embodiments, the sample further comprises a machine-readable label such as a barcode. In some embodiments, the label comprising encoded information relating to the sample shape, sample size, sample type, sample orientation, organism from which the sample was obtained, number of samples in proximity to the label, or instructions for further data analysis.

In some embodiments, the sample undergoes a nucleic acid amplification reaction prior to contacting the photons. In some embodiments, the nucleic acid amplification reaction is a loop mediated amplification (LAMP) reaction. In some embodiments, the nucleic acid amplification reaction is a PCR reaction. In some embodiments, the method is performed at about or at a temperature range of 55-65° C. In some embodiments, at least a portion of the sample is partitioned into an array comprising at least 2 or more containers, wherein the image comprises optical data from the location of each container. In some embodiments, the optical data is a fluorescent signal or a lack of a fluorescent signal. In some embodiments, the array is a SlipChip. In some embodiments, the nucleic acid that is amplified is RNA.

In some embodiments, the analysis of the digital quantification of nucleic acids within a sample yields a consistent conclusion description for the sample for at least one of the reaction parameters selected from the group consisting of: i) reaction occurs in a temperature range between 57° C. and 63° C.; ii) reaction time between 15 min and 1.5 hours; iii) humidity is between 0% and 100%; and iv) background light is between 0 and 6 lux. In some embodiments, the consistent conclusion description for the sample for at least two of the reaction parameters. In some embodiments, the consistent conclusion description for the sample for at least three of the reaction parameters. In some embodiments, the consistent conclusion description for the sample for four of the reaction parameters. In some embodiments, wherein the image sensor is part of a cell phone or tablet computer.

In some embodiments, the method further comprises at least one of the following steps: detection of a fluorescent region using a cell phone; detection of a fluorescent region using a mobile handheld device; detection of a fluorescent region corresponding to an amplification product from a single molecule; exciting fluorescence using a compact flash integrated with a mobile communication device; transmitting an image and/or a processed image and/or resulting data to a centralized computer; background correction of an image using a combination of color channels; enhancement of fluorescent regions by using one or more filtering algorithms; shape detection using one or more shapes to determine image fidelity; shape detection using one or more shapes to determine the region to be analyzed; shape detection using one or more algorithms to determine positive regions; processing and/or analyzing images and/or data on the centralized computer; optionally archiving the images and/or data; transmitting information back to the mobile device; transmitting an image and/or a processed image and/or resulting data the user; transmitting an image and/or a processed image and/or resulting data to a third party; applying Poisson statistical analysis to quantify the number of fluorescent and non-fluorescent regions; applying Poisson statistical analysis to quantify concentration based on the number of fluorescent and non-fluorescent regions.

In some embodiments, the light source has a light intensity of at least greater or equal to 100,000 lux.

In some embodiments, the light is emitted from a mobile phone containing a built-in camera or is a tablet containing a built-in camera.

In some embodiments, the light it filtered.

In some embodiments, the filter comprises a set of filters.

In some embodiments, the set of filters comprises at least one, two, three, four filters or any combination thereof. In some embodiments, the filters comprises a fluorescent filter. In some embodiments, the fluorescent filter comprises a dichroic filter and/or a long-pass filter. In some embodiments, the dichroic filter can be greater than 85% transmission about or at 390-480 nm and less than 1% about or at 540-750 nm. In some embodiments, the long-pass filter can have blocking of greater than 5 OD and transmission of greater than 90% at wavelengths about or at 530-750 nm.

In some embodiments, the analysis process can take less than one minute.

In some embodiments, the analysis process performs a background correction of an image using a data collected from a second color channel. In some embodiments, the software algorithm can apply Poisson statistical analysis to quantify the number of fluorescent and non-fluorescent regions.

In some embodiments, the data analysis takes place locally, through a cloud-based service, through a centralized computer located remotely or any combination thereof.

In some embodiments, the method is providing an application for detecting nucleic acids.

In some embodiments, the portable digital device is tilted at an angled position when taking a picture.

In another aspect, the invention provides a device for generating sample data comprising: i) a light source that emits a set of photons in a short burst, the burst lasting from about 5/1,000,000 seconds to about one second, wherein at least a portion of the photons contact the sample; ii) an image sensor not in alignment with the light source that collects at least a portion of the photons that contacted the sample to create data associated with the sample; iii) a processor configured to process the sample data to create a binary quantification of nucleic acids in the sample or a wireless connection to transmit the sample data to a different device configured to create a binary quantification of nucleic acids in the sample; and iv) a processor configured to analyze the binary quantification of nucleic acids to generate a conclusion description relating to the sample.

In some embodiments, the device further comprises a filter. In some embodiments, the set of filters comprises at least one, two, three, four filters or any combination thereof. In some embodiments, the filters comprises a fluorescent filter. In some embodiments, the fluorescent filter comprises a dichroic filter and/or a long-pass filter. In some embodiments, the dichroic filter can be greater than 85% transmission about or at 390-480 nm and less than 1% about or at 540-750 nm. In some embodiments, the long-pass filter can have blocking of greater than 5 OD and transmission of greater than 90% at wavelengths about or at 530-750 nm.

In some embodiments the device comprises a screen to display the conclusion description.

In some embodiments of the device, the light source is a camera flash.

In some embodiments of the device, the image sensor is CCD or CMOS.

In yet another aspect, the invention provides a kit comprising a container comprising: i) a plurality of small containers; ii) components of a nucleic acid amplification reaction; iii) and instructions for use. In some embodiments, the plurality of small containers is a SlipChip.

In some embodiments, the kit further comprises a machine-readable label such as a barcode. In some embodiments, the label comprising encoded information relating to the sample shape, sample size, sample type, sample orientation, organism from which the sample was obtained, number of samples in proximity to the label, or instructions for further data analysis. In some embodiments of the kit, the components of a nucleic acid amplification reaction are located within at least one of the small containers. In some embodiments the kits described herein, further comprise a device described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

The present application incorporates the following applications by reference in their entireties for any and all purposes: U.S. Application 61/516,628, “Digital Isothermal Quantification of Nucleic Acids Via Simultaneous Chemical Initiation of Recombinase Polymerase Amplification (RPA) Reactions on Slip Chip,” filed on Apr. 5, 2011; U.S. Application 61/518,601, “Quantification of Nucleic Acids With Large Dynamic Range Using Multivolume Digital Reverse Transcription PCR (RT-PCR) On A Rotational Slip Chip Tested With Viral Load,” filed on May 9, 2011; U.S. application Ser. No. 13/257,811, “Slip Chip Device and Methods,” filed on Sep. 20, 2011; international application PCT/US2010/028361, “Slip Chip Device and Methods,” filed on Mar. 23, 2010; U.S. Application 61/262,375, “Slip Chip Device and Methods,” filed on Nov. 18, 2009; U.S. Application 61/162,922, “Sip Chip Device and Methods,” filed on Mar. 24, 2009; U.S. Application 61/340,872, “Slip Chip Device and Methods,” filed on Mar. 22, 2010; U.S. application Ser. No. 13/440,371, “Analysis Devices, Kits, And Related Methods For Digital Quantification Of Nucleic Acids And Other Analytes,” filed on Apr. 5, 2012; and U.S. application Ser. No. 13/467,482, “Multivolume Devices, Kits, and Related Methods for Quantification and Detection of Nucleic Acids and Other Analytes,” filed on May 9, 2012.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates the robustness of quantification in digital vs. kinetic formats. Cartoons for the curves in the kinetic format are drawn to resemble a specific case of real-time nucleic acid amplification. FIG. 1a compares digital and kinetic formats under ideal conditions. In a digital format, individual molecules are separated into compartments and amplified, requiring only an end-point readout. The original concentration (C) of the analyte can be calculated by the equation on the left (where wₚ=the number of positive wells, wₜ=the total number of wells, and wᵥ=the volume of each well). In a kinetic format, the analyte is amplified in a bulk culture and the progress of amplification, measured as intensity, is monitored as a function of time. The original concentration is determined by comparing the reaction trace to standard curves from solutions of known concentration. FIG. 1b illustrates the effects of kinetic variation (shown as differences in amplification temperature) in digital and real-time formats. In a digital format, the variance in the kinetic rate of amplification would potentially not affect the end-point readout. In a real-time format, the kinetic rate determines the reaction curve and thus the relative concentration; therefore, it is known to be not robust. FIG. 1c illustrates the effects of time variance (shown as readout time) in digital and real-time formats. Since digital requires only end-point readout, the exact knowledge of time would not be necessarily required and the output should be robust to variation in reaction time beyond the optimal reaction time. In a real-time format, precise knowledge of time and sufficient time points are required in order to accurately quantify concentration; therefore, it is known to be not robust to variation in reaction time. FIG. 1d illustrates the effects of imaging in digital and real-time formats. In a real-time format, imaging conditions with increased noise or decreased sensitivity can affect quantitative ability by producing reaction traces that cannot be compared to standards; therefore, it is known to be not robust to variation in imaging conditions.

FIG. 2 illustrates an evaluation of the robustness of real-time RT-LAMP versus digital RT-LAMP with respect to changes in temperature, time, and imaging conditions. FIG. 2a-b illustrate the results of real-time RT-LAMP experiments (2a) and digital RT-LAMP experiments (2b) for two concentrations across a 6-degree temperature range. Imaging was performed with a microscope. FIG. 2c illustrates the number of positive counts from dRT-LAMP experiments for two concentrations at various reaction times. FIG. 2d illustrates a plot comparing the data obtained from imaging with a microscope in part (2b), data obtained from imaging dRT-LAMP with a cell phone in a shoebox, and data obtained from imaging dRT-LAMP in dim lighting (˜3 lux) across a 6-degree temperature range. P-values denote statistical significance of all data for each concentration at a given imaging condition, irrespective of temperature (the null hypothesis being that the two concentrations were equivalent). FIG. 2e illustrates cropped and enlarged images of a dRT-LAMP reaction imaged with a microscope (top) and its corresponding line scan indicating fluorescence output from the region marked in white (bottom). FIG. 2f illustrates a cell phone and shoe box (top) and its corresponding line scan indicating fluorescence output from the region marked in white (bottom). FIG. 2g illustrates a cell phone in dim lighting (top) and its corresponding line scan indicating fluorescence output from the region marked in white (bottom). The number of positives in each dRT-LAMP experiment imaged with a cell phone was counted manually. Error bars represent standard deviation.

FIG. 3 illustrates cell phone imaging of multiplexed PCR on a SlipChip device using five different primer sets and a single template. FIG. 3a illustrates a schematic drawing of a SlipChip device that has been pre-loaded with primers. FIG. 3b illustrates a schematic drawing showing the arrangement of the five primer sets on the device: 1=E. coli nlp gene, 2=P. aeruginosa vic gene, 3=C. albicans calb gene, 4=Pseudomonas 16S, 5=S. aureus nuc gene. FIG. 3c illustrates a cell phone image of a SlipChip after loading it with S. aureus genomic DNA and performing PCR amplification. Wells containing the primer for S. aureus increased in fluorescence to form the designed pattern. The intensity levels of the image have been adjusted and the image has been smoothed to enhance printed visibility.

FIG. 4 illustrates images of the device with PCR reaction outcomes taken by a cell phone before (top left) and after (top right) image processing and line scans showing gray values as a function of distance in pixels for before image processing (bottom left) and after image processing (bottom right).

FIG. 5 illustrates the robustness of digital dRT-LAMP amplification imaged with a microscope to thresholding. FIG. 5a illustrates a graph showing the number of positive reactions observed when imaging the dRT-LAMP reactions with a microscope compared to the threshold value used to calculate the number of positives. FIG. 5b illustrates a plot of the p-values generated by comparing the two concentrations at threshold values between 90 and 240. The minimum p-value is observed at a threshold of 190.

FIG. 6 illustrates the image analysis workflow used to count molecules via digital amplification with a SlipChip and a cell phone. FIG. 6a illustrates a cell phone and a device labeled with four dark circles that the imaging processing algorithm uses to confirm that the entire device has been imaged. FIG. 6b illustrates a cartoon representation of a cloud-based server that analyzes photographs taken by the user, archives the raw data, and sends the results to the appropriate party. FIG. 6c illustrates screenshots of a cell phone screen showing email messages received by a pre-specified recipient after analysis of successful (top left) and unsuccessful (bottom left) imaging and the successful image that was analyzed (top right) and unsuccessful image that was analyzed (bottom right). FIG. 6d illustrates a graph comparing the raw positive counts processed from a cell phone (y-axis) and thresholding performed with an epifluorescence microscope (x-axis).

FIG. 7 illustrates schematic drawings and images showing the operation of SlipChip for two-step dRT-LAMP. FIG. 7a illustrates the top and bottom plates of the SlipChip before assembly. FIG. 7b illustrates an assembled SlipChip after loading of RT solution. FIG. 7c illustrates RT solution containing RNA molecules confined to individual wells after slipping. FIG. 7d illustrates loading of LAMP reagent mixture after RT reaction has completed. FIG. 7e illustrates LAMP reagent mixture confined to individual wells after slipping again. FIG. 7f illustrates reaction initiated after slipping to mix RT and LAMP wells.

FIG. 8a illustrates the concentration of HIV viral RNA (copies/mL) measured with dRT-LAMP using different protocols and the same template concentration. i) one-step dRT-LAMP; ii) two-step dRT-LAMP, all primers in RT step, AMV RT; iii) two-step, BIP in RT step, AMV RT; iv) two-step, BIP in RT step, Superscript III; v) two-step, BIP in RT step, AMV RT, with RNase H; vi) two-step, BIP in RT step, Superscript III, with RNase H; vii) two-step, BIP in RT step, Superscript III, with RNase H, 0.5× calcein. FIG. 8b illustrates quantification results of HIV viral RNA (copies/mL) with the second step performed at different temperatures. FIG. 8c illustrates quantification results of HIV viral RNA (copies/mL) on a plastic SlipChip at two concentrations, with comparisons to results obtained on a glass device. (n=2 in all experiments, error bars represent standard deviation.)

FIG. 9 illustrates quantification of HIV viral RNA purified from patient samples using dRT-LAMP and dRT-PCR. For sample #4, quantification results using dRT-LAMP with corrected primers are shown in the rightmost column of the figure. (n=2 in all experiments, error bars represent standard deviation.)

### DETAILED DESCRIPTION OF THE INVENTION

Unless defined otherwise, all technical and scientific terms used herein have the same meaning as is commonly understood by one of skill in the art to which the claimed subject matter belongs. All patents, patent applications, published applications and publications, GENBANK sequences, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it is understood that such identifiers can change and particular information on the internet can come and go, but equivalent information is known and can be readily accessed, such as by searching the internet and/or appropriate databases. Reference thereto evidences the availability and public dissemination of such information.

As used herein, the singular forms “a,” “an” and “the” include plural referents unless the context clearly dictates otherwise. In this application, the use of the singular includes the plural unless specifically stated otherwise. As used herein, the use of “or” means “and/or” unless stated otherwise. Furthermore, use of the term “including” as well as other forms (e.g., “include”, “includes”, and “included”) is not limiting.

As used herein, ranges and amounts can be expressed as “about” a particular value or range. About also includes the exact amount. Hence “about 10 degrees” means “about 10 degrees” and also “10 degrees.” Generally, the term “about” can include an amount that would be expected to be within experimental error.

Disclosed herein are methods, devices and systems related to detection of diseases or organisms. The detection can be detection of a signal generated by an assay, for example, an assay to detect a nucleic acid associated with a disease or organism. In some embodiments the signal is detected by a consumer grade camera, for example a camera on a cell phone.

The term “organism” refers to any organisms or microorganism, including bacteria, yeast, fungi, viruses, protists (protozoan, micro-algae), archaebacteria, plants and eukaryotes. The term “organism” refers to living matter and viruses comprising nucleic acid that can be detected and identified by the methods of the invention. Organisms include, but are not limited to, bacteria, archaea, prokaryotes, eukaryotes, viruses, protozoa, mycoplasma, fungi, plants and nematodes. Different organisms can be different strains, different varieties, different species, different genera, different families, different orders, different classes, different phyla, and/or different kingdoms. Organisms may be isolated from environmental sources including soil extracts, marine sediments, freshwater sediments, hot springs, ice shelves, extraterrestrial samples, crevices of rocks, clouds, attached to particulates from aqueous environments, and may be involved in symbiotic relationships with multicellular organisms. Examples of such organisms include, but are not limited to Streptomyces species and uncharacterized/unknown species from natural sources.

Organisms can include genetically engineered organisms or genetically modified organisms.

Organisms can include transgenic plants. Organisms can include genetically modified crops. Any organism can be genetically modified. Examples of organisms which can be genetically modified include plantains, yams, sorghum, sweet potatoes, soybeans, cassava, potatoes, rice, wheat, or corn.

Organisms can include bacterial pathogens such as: Aeromonas hydrophile and other species (spp.); Bacillus anthracis; Bacillus cereus; Botulinum neurotoxin producing species of Clostridium; Brucella abortus; Brucella melitensis; Brucella suis; Burkholderia mallei (formally Pseudomonas mallei); Burkholderia pseudomallei (formerly Pseudomonas pseudomallei); Campylobacter jejuni; Chlamydia psittaci; Clostridium botulinum; Clostridium botulinum; Clostridium perfringens; Coccidioides immitis; Coccidioides posadasii; Cowdria ruminantium (Heartwater); Coxiella burnetii; Enterovirulent Escherichia co//group (EEC Group) such as Escherichia coli—enterotoxigenic (ETEC), Escherichia coli—enteropathogenic (EPEC), Escherichia coli-O157:H7 enterohemorrhagic (EHEC), and Escherichia coli—enteroinvasive (EIEC); Ehrlichia spp. such as Ehrlichia chaffeensis; Francisella tularensis; Legionella pneumophilia; Liberobacter africanus; Liberobacter asiaticus; Listeria monocytogenes; miscellaneous enterics such as Klebsiella, Enterobacter, Proteus, Citrobacter, Aerobacter, Providencia, and Serratia; Mycobacterium bovis; Mycobacterium tuberculosis; Mycoplasma capricolum; Mycoplasma mycoides ssp mycoides; Peronosclerospora philippinensis; Phakopsora pachyrhizi; Plesiomonas shigelloides; Ralstonia solanacearum race 3, biovar 2; Rickettsia prowazekii; Rickettsia rickettsii; Salmonella spp.; Schlerophthora rayssiae varzeae; Shigella spp.; Staphylococcus aureus; Streptococcus; Synchytrium endobioticum; Vibrio cholerae non-O1; Vibrio cholerae O1; Vibrio parahaemolyticus and other Vibrios; Vibrio vulnificus; Xanthomonas oryzae; Xylella fastidiosa (citrus variegated chlorosis strain); Yersinia enterocolitica and Yersinia pseudotuberculosis; and Yersinia pestis.

Organisms can include viruses such as: African horse sickness virus; African swine fever virus; Akabane virus; Avian influenza virus (highly pathogenic); Bhanja virus; Blue tongue virus (Exotic); Camel pox virus; Cercopithecine herpesvirus 1; Chikungunya virus; Classical swine fever virus; Coronavirus (SARS); Crimean-Congo hemorrhagic fever virus; Dengue viruses; Dugbe virus; Ebola viruses; Encephalitic viruses such as Eastern equine encephalitis virus, Japanese encephalitis virus, Murray Valley encephalitis, and Venezuelan equine encephalitis virus; Equine morbillivirus; Flexal virus; Foot and mouth disease virus; Germiston virus; Goat pox virus; Hantaan or other Hanta viruses; Hendra virus; Issyk-kul virus; Koutango virus; Lassa fever virus; Louping ill virus; Lumpy skin disease virus; Lymphocytic choriomeningitis virus; Malignant catarrhal fever virus (Exotic); Marburg virus; Mayaro virus; Menangle virus; Monkeypox virus; Mucambo virus; Newcastle disease virus (WND); Nipah Virus; Norwalk virus group; Oropouche virus; Orungo virus; Peste Des Petits Ruminants virus; Piry virus; Plum Pox Potyvirus; Poliovirus; Potato virus; Powassan virus; Rift Valley fever virus; Rinderpest virus; Rotavirus; Semliki Forest virus; Sheep pox virus; South American hemorrhagic fever viruses such as Flexal, Guanarito, Junin, Machupo, and Sabia; Spondweni virus; Swine vesicular disease virus; Tickborne encephalitis complex (flavi) viruses such as Central European tickborne encephalitis, Far Eastern tick-borne encephalitis, Russian spring and summer encephalitis, Kyasanur forest disease, and Omsk hemorrhagic fever; Variola major virus (Smallpox virus); Variola minor virus (Alastrim); Vesicular stomatitis virus (Exotic); Wesselbron virus; West Nile virus; Yellow fever virus; and South American hemorrhagic fever viruses such as Junin, Machupo, Sabia, Flexal, and Guanarito.

Further examples of organisms include parasitic protozoa and worms, such as: Acanthamoeba and other free-living amoebae; Anisakis sp. and other related worms Ascaris lumbricoides and Trichuris trichiura; Cryptosporidium parvum; Cyclospora cayetanensis; Diphyllobothrium spp.; Entamoeba histolytica; Eustrongylides sp.; Giardia lamblia; Nanophyetus spp.; Shistosoma spp.; Toxoplasma gondii; Filarial nematodes and Trichinella. Further examples of analytes include allergens such as plant pollen and wheat gluten.

Further examples of organisms include fungi such as: Aspergillus spp.; Blastomyces dermatitidis; Candida; Coccidioides immitis; Coccidioides posadasii; Cryptococcus neoformans; Histoplasma capsulatum; Maize rust; Rice blast; Rice brown spot disease; Rye blast; Sporothrix schenckii; and wheat fungus. Further examples of organisms include worms such as C. Elegans and pathogenic worms or nematodes.

The term “disease” refers to any state, condition, or characteristic which may be considered abnormal to an organism. A disease can be a medical condition. A disease can be a disorder. A disease can be associated with a set of symptoms. A disease can be communicable. A disease can be non-communicable. The term disease can, in some embodiments, also include risk factors for a disease or a pre-disease.

A disease can be chronic. A disease can be acute. A disease can have flare-ups or reoccurrences. In some embodiments the methods, devices and systems provided herein can detect a disease state, for example an active phase of a disease or an amount of a viral load associated with a disease. In some embodiments, diseases caused by virus include HIV/AIDS, malaria, measles, diarrheal diseases and respiratory infections.

The disease can be a genetic. A genetic disease can be associated with a single gene. A genetic disease can be associated with multiple genes. A genetic disorder can be associated with a single nucleotide polymorphism. Some non-limiting examples of a genetic disorder include the following.

Genetic diseases that can be tested according to this invention include, but are not limited to: 21-Hydroxylase Deficiency, ABCC8-Related Hyperinsulinism, ARSACS, Achondroplasia, Achromatopsia, Adenosine Monophosphate Deaminase 1, Agenesis of Corpus Callosum with Neuronopathy, Alkaptonuria, Alpha-1-Antitrypsin Deficiency, Alpha-Mannosidosis, Alpha-Sarcoglycanopathy, Alpha-Thalassemia, Alzheimers, Angiotensin II Receptor, Type 1, Apolipoprotein E Genotyping, Argininosuccinicaciduria, Aspartylglycosaminuria, Ataxia with Vitamin E Deficiency, Ataxia-Telangiectasia, Autoimmune Polyendocrinopathy Syndrome Type 1, BRCA1 Hereditary Breast/Ovarian Cancer, BRCA2 Hereditary Breast/Ovarian Cancer, Bardet-Biedl Syndrome, Best Vitelliform Macular Dystrophy, Beta-Sarcoglycanopathy, Beta-Thalassemia, Biotinidase Deficiency, Blau Syndrome, Bloom Syndrome, CFTR-Related Disorders, CLN3-Related Neuronal Ceroid-Lipofuscinosis, CLN5-Related Neuronal Ceroid-Lipofuscinosis, CLN8-Related Neuronal Ceroid-Lipofuscinosis, Canavan Disease, Carnitine Palmitoyltransferase IA Deficiency, Carnitine Palmitoyltransferase II Deficiency, Cartilage-Hair Hypoplasia, Cerebral Cavernous Malformation, Choroideremia, Cohen Syndrome, Congenital Cataracts, Facial Dysmorphism, and Neuropathy, Congenital Disorder of Glycosylationla, Congenital Disorder of Glycosylation Ib, Congenital Finnish Nephrosis, Crohn Disease, Cystinosis, DFNA 9 (COCH), Diabetes and Hearing Loss, Early-Onset Primary Dystonia (DYT1), Epidermolysis Bullosa Junctional, Herlitz-Pearson Type, FANCC-Related Fanconi Anemia, FGFR1-Related Craniosynostosis, FGFR2-Related Craniosynostosis, FGFR3-Related Craniosynostosis, Factor V Leiden Thrombophilia, Factor V R2 Mutation Thrombophilia, Factor XI Deficiency, Factor XIII Deficiency, Familial Adenomatous Polyposis, Familial Dysautonomia, Familial Hypercholesterolemia Type B, Familial Mediterranean Fever, Free Sialic Acid Storage Disorders, Frontotemporal Dementia with Parkinsonism-17, Fumarase deficiency, GJB2-Related DFNA 3 Nonsyndromic Hearing Loss and Deafness, GJB2-Related DFNB 1 Nonsyndromic Hearing Loss and Deafness, GNE-Related Myopathies, Galactosemia, Gaucher Disease, Glucose-6-Phosphate Dehydrogenase Deficiency, Glutaricacidemia Type 1, Glycogen Storage Disease Type Ia, Glycogen Storage Disease Type Ib, Glycogen Storage Disease Type π, Glycogen Storage Disease Type HI, Glycogen Storage Disease Type V, Gracile Syndrome, HFE-Associated Hereditary Hemochromatosis, Haider AIMs, Hemoglobin S Beta-Thalassemia, Hereditary Fructose Intolerance, Hereditary Pancreatitis, Hereditary Thymine-Uraciluria, Hexosaminidase A Deficiency, Hidrotic Ectodermal Dysplasia 2, Homocystinuria Caused by Cystathionine Beta-Synthase Deficiency, Hyperkalemic Periodic Paralysis Type 1, Hyperornithinemia-Hyperammonemia-Homocitrullinuria Syndrome, Hyperoxaluria, Primary, Type 1, Hyperoxaluria, Primary, Type 2, Hypochondroplasia, Hypokalemic Periodic Paralysis Type 1, Hypokalemic Periodic Paralysis Type 2, Hypophosphatasia, Infantile Myopathy and Lactic Acidosis (Fatal and Non-Fatal Forms), Isovaleric Acidemias, Krabbe Disease, LGMD2I, Leber Hereditary Optic Neuropathy, Leigh Syndrome, French-Canadian Type, Long Chain 3-Hydroxyacyl-CoA Dehydrogenase Deficiency, MELAS, MERRF, MTHFR Deficiency, MTHFR Thermolabile Variant, MTRNR1-Related Hearing Loss and Deafness, MTTS1-Related Hearing Loss and Deafness, MYH-Associated Polyposis, Maple Syrup Urine Disease Type IA, Maple Syrup Urine Disease Type IB, McCune-Albright Syndrome, Medium Chain Acyl-Coenzyme A Dehydrogenase Deficiency, Megalencephalic Leukoencephalopathy with Subcortical Cysts, Metachromatic Leukodystrophy, Mitochondrial Cardiomyopathy, Mitochondrial DNA-Associated Leigh Syndrome and NARP, Mucolipidosis IV, Mucopolysaccharidosis Type I, Mucopolysaccharidosis Type IHA, Mucopolysaccharidosis Type Vπ, Multiple Endocrine Neoplasia Type 2, Muscle-Eye-Brain Disease, Nemaline Myopathy, Neurological phenotype, Niemann-Pick Disease Due to Sphingomyelinase Deficiency, Niemann-Pick Disease Type Cl, Nijmegen Breakage Syndrome, PPT1-Related Neuronal Ceroid-Lipofuscinosis, PROP1-related pituitary hormome deficiency, Pallister-Hall Syndrome, Paramyotonia Congenita, Pendred Syndrome, Peroxisomal Bifunctional Enzyme Deficiency, Pervasive Developmental Disorders, Phenylalanine Hydroxylase Deficiency, Plasminogen Activator Inhibitor I, Polycystic Kidney Disease, Autosomal Recessive, Prothrombin G20210A Thrombophilia, Pseudovitamin D Deficiency Rickets, Pycnodysostosis, Retinitis Pigmentosa, Autosomal Recessive, Bothnia Type, Rett Syndrome, Rhizomelic Chondrodysplasia Punctata Type 1, Short Chain Acyl-CoA Dehydrogenase Deficiency, Shwachman-Diamond Syndrome, Sjogren-Larsson Syndrome, Smith-Lemli-Opitz Syndrome, Spastic Paraplegia 13, Sulfate Transporter-Related Osteochondrodysplasia, TFR2-Related Hereditary Hemochromatosis, TPP1-Related Neuronal Ceroid-Lipofuscinosis, Thanatophoric Dysplasia, Transthyretin Amyloidosis, Trifunctional Protein Deficiency, Tyrosine Hydroxylase-Deficient DRD, Tyrosinemia Type I, Wilson Disease, X-Linked Juvenile Retinoschisis and Zellweger Syndrome Spectrum.

Disclosed herein are methods, devices and systems related to analysis of samples. A sample can be obtained from a patient or person and includes blood, feces, urine, saliva or other bodily fluid. Food samples may also be analyzed. Samples may be any composition potentially comprising an organism. Samples may be any composition potentially comprising a nucleic acid, for example a nucleic acid related to a disease or organism. Samples may be any composition comprising substances related to disease. Sources of samples include, but are not limited to, geothermal and hydrothermal fields, acidic soils, sulfotara and boiling mud pots, pools, hot-springs and geysers where the enzymes are neutral to alkaline, marine actinomycetes, metazoan, endo and ectosymbionts, tropical soil, temperate soil, arid soil, compost piles, manure piles, marine sediments, freshwater sediments, water concentrates, hypersaline and super-cooled sea ice, arctic tundra, Sargasso sea, open ocean pelagic, marine snow, microbial mats (such as whale falls, springs and hydrothermal vents), insect and nematode gut microbial communities, plant endophytes, epiphytic water samples, industrial sites and ex situ enrichments. Additionally, a sample may be isolated from eukaryotes, prokaryotes, myxobacteria (epothilone), air, water, sediment, soil or rock, a plant sample, a food sample, a gut sample, a salivary sample, a blood sample, a sweat sample, a urine sample, a spinal fluid sample, a tissue sample, a vaginal swab, a stool sample, an amniotic fluid sample, a fingerprint, aerosols, including aerosols produced by coughing, skin samples, tissues, including tissue from biopsies, and/or a buccal mouthwash sample.

Samples can be collected in a sample collection container. In some embodiments the sample collection container is coded with information that can be detected. For example a detector may recognize a barcode. The barcode can have information about where a sample was collected or from which individual a sample was collected. A detector may take this information and use it to process or transmit data generated regarding a sample. For example a camera-phone may take a photo of a sample collection container. The camera-phone can recognize a barcode on the container which identifies a patient. The camera-phone can then link date generated regarding the sample to the patient from which the sample was obtained. The linked data can then be transmitted to the patient or to the patient's physician. In some embodiments a single image is generated of the sample collection container and a sample analysis unit.

In some embodiments, methods of the invention comprises obtaining a sample from a subject. The sample can be obtained by the subject or by a medical professional. Examples of medical professionals include, but are not limited to, physicians, emergency medical technicians, nurses, first responders, psychologists, medical physics personnel, nurse practitioners, surgeons, dentists, and any other medical professional. The sample can be obtained from any bodily fluid, for example, amniotic fluid, aqueous humor, bile, lymph, breast milk, interstitial fluid, blood, blood plasma, cerumen (earwax), Cowper's fluid (pre-ejaculatory fluid), chyle, chyme, female ejaculate, menses, mucus, saliva, urine, vomit, tears, vaginal lubrication, sweat, serum, semen, sebum, pus, pleural fluid, cerebrospinal fluid, synovial fluid, intracellular fluid, and vitreous humour. In an example, the sample is obtained by a blood draw, where the medical professional draws blood from a subject, such as by a syringe. The bodily fluid can then be tested to determine the prevalence of the biomarker. Biological markers, also referred to herein as biomarkers, according to the present invention include without limitation drugs, prodrugs, pharmaceutical agents, drug metabolites, biomarkers such as expressed proteins and cell markers, antibodies, serum proteins, cholesterol, polysaccharides, nucleic acids, biological analytes, biomarker, gene, protein, or hormone, or any combination thereof. At a molecular level, the biomarkers can be polypeptide, glycoprotein, polysaccharide, lipid, nucleic acid, and a combination thereof.

Disclosed herein are methods, devices and systems which can employ light sources for the analysis of samples. The light source may emit photons in the visual spectrum. The light source may emit photons in the UV spectrum. The light source may emit photons in the IR spectrum. The light source may emit photons of any wavelength. In some embodiments, the light source is a Xenon light source. In some embodiments, the light source is an LED. In some embodiments the light source is not an arc lamp.

The light source can be a flash. The flash can be an air-gap flash. The flash can be an a multi-flash. In some embodiments a multiflash is used to create multiple images for subsequent analysis.

The light source can have a brief duration. The brief duration can be for example about 0.0001, about 0.001, about 0.01, about 0.1, or about 1 second.

The light source can produce an unstabilized light. Unstabilized light can be light that has a parameter changing over time. For example the intensity of the light emitted from the source may be changing over time. For example the wavelength of the light emitted from the source may be changing over time. In some embodiments photons are collected by an image detector during a time when the light source is producing unstabilized light. In some embodiments a sample is imaged using unstabalized light.

The light source, in some embodiments, can produce stabilized light. Stabilized light can be light that has a parameter that is not changing over time. For example a stabilized light can emit light with an intensity that is not significantly changing over time.

A light source can comprise ambient light. A light source can also be combined with ambient light. In some embodiments ambient light comprises less that 10%, less than 5%, less than 1%, or less than 0.1% of the photons reaching a sample prior to analysis.

The light source can be battery operated.

The light source can be not in line with the image sensor. For example the light source can be a flash located on a cell phone camera. In some embodiments the light source is not located between a sample and an image sensor. In some embodiments the light source is closer to the image sensor than it is to the sample. In some embodiments the light source is at least 10 times, 50 times, or 100 times, closer to the image sensor that it is to the sample.

The light source can be non-stabilized during data gathering. For example, a detector may be collecting photos as a parameter of the light source shifts. Examples of the shifting parameter can be light intensity or wavelength. The parameter can shift more than 1%, 5%, 10%, 20%, 30%, 40%, 50%, 60%, 70%, 80%, 90%, or 99%. In one example a detector is collecting data during a flash and until after the flash ends. In one example, a detector is collecting data before a flash begins and during the flash.

The light source can be contained within a separate device. In some embodiments, the separate device can be separately powered and capable of providing both excitation light to visualize an assay outcome, and heat to run an amplification. In some examples, the device can be self contained to block any unwanted external light. In some examples, the device may contain a specific location and/or holder to position the sample. In one example the device may contain a specific location and/or holder to position the imaging device. In one example the device may use LED, compact fluorescent lamp, mercury lamp, incandescent lamp, etc for example.

Filters can be placed between the light source and the sample. A single filter can be used. Multiple filters can also be used. In some instances the filter or filters are physically connected to the light source. In some embodiments the filter or filters are physically connected to an image sensor. The physical connection can be indirect, for example the filter can be connected to a housing which contains the image sensor.

A filter can be a bandpass filter. Optical Bandpass Filters can be used, e.g., to selectively transmit a portion of the spectrum while rejecting all other wavelengths. A filter can have, e.g., bandwidths of about 1000-1650 nm, 200-400 nm, 500-500 nm, 500-600 nm, or 20-70 nm. A filter can be Multi-Band Fluorescence Bandpass Filters.

A filter can be a longpass edge filter. Longpass Edge Filters can, e.g., transmit wavelengths greater than the cut-on wavelength of the filter.

A filter can be a shortpass edge filter. Shortpass Edge Filters can, e.g., transmit wavelengths shorter than the cut-off wavelength of the filter.

A filter can be a notch filter. A notch filter can, e.g., reject a portion of the spectrum, while transmitting all other wavelengths.

A filter can be a neutral density filter. A filter can be an imaging filter. A filter can be a diachronic or color filter. For example, dichroic filters 1F1B (Thorlabs, Newton, N.J.) can be placed in front of a flash light source. These filters can have for example >85% transmission for 390-480 nm and <1% for 540-750 nm, with a cut-off of 505±15 nm. The filters can be added to an objective lens. For example, green long-pass 5CGA-530 filters from Newport (Franklin, Mass.) can be added to an objective lens. These filters block, for example, have >5OD and high transmission of >90% at wavelengths over 530 nm.

The sample can be imaged in a container or sample containing device. The sample containing device can have a geometry that provides for an optimal imaging orientation. In some embodiments the image sensor is aligned optimally—such that the best possible image is captured of the sample. In some embodiments the image sensor is sub optimally orientated. For example the image sensor may be tilted or skewed with respect to the optimal alignment.

Software can be used to determine whether the degree of suboptimal alignment is within the tolerance of the device. For example an image of a suboptimally aligned device may be analyzed to determine whether the image is within a known tolerance of the device. In some embodiments, an accelerometer or gravity sensor within a cell phone, for example an iPhone, senses the alignment of the image sensor, and an image is collected when a tolerated image sensor alignment relative to the sample is achieved. In some embodiments the alignment is determined by generating a first image of a sample containing device of known size, shape or with indicators in a known orientation. The device can then calculate the geometry based on these known parameters and determine. The device can then determine whether the image sensor can successfully generate data. In some embodiments these tolerances are adjusted according to the amount of ambient light, the surface or the sample containing device, or based on the success or failure of previous imaging attempts. In some embodiments the user can input values which affect the tolerance calculations of the device. For example a user can increase a stringency which would cause the device to have a lower tolerance for sub optimal alignment.

The orientation of the device may also be altered to compensate for properties of the sample containing device. For example in some embodiments, a sample containing device is reflective, and can be tilted by about and or 0 degrees, about and or 10 degrees, about and or 20 degrees, about and or 30 degrees, about and or 40 degrees, about and or 50 degrees or about and or 60 degrees relative to an image sensor-device axis. This tilt can prevent direct reflection back to the objective and to force direct reflected light to go to the side due to tilt. In some embodiments the tilt is in multiple planes.

Additional components can be added to compensate for sub-optimal alignment, for example a black screen can be added on the side of the device to block the scattered light from flash from oversaturating the CMOS sensor.

Such geometry, and screens, combined with the filters described above, allows reaching signal to noise ratios of about 50. Signal to noise ratios can be calculated by the device and can be about 10, 20, 30, 40, 50, 60, 70, 80, or 90, depending on the particular application.

The sample containment device, in some embodiments, is not in physical communication with the image sensor. For example, the image sensor may be hand-held while the sample containment device is on a surface.

In some embodiments feedback is provided to a user to inform the user that the image sensor is positioned correctly for successful imaging. For example a phone based camera can detect a sample or sample carrier and provided feedback to a user when the sample or sample carrier is within a tolerate of the device. For example a “ready” signal may be sent to the user.

Photons that have interacted with the sample can be collected using an image sensor. The image sensor can comprise one or more sensors. The image sensor can comprise, for example, a CCD, CMOS, or a CCD/CMOS hybrid

The device can be configured for color separation. For example the image sensor can have multiple filtered pixels. A CCD can have, for example, a Bayer mask. Alternatives to the Bayer filter include various modifications of colors, various modifications of arrangement, and completely different technologies, such as color co-site sampling, the Foveon X3 sensor or dichroic mirrors. In some embodiments a three-CCD device is the image sensor.

The device can record a signal from a sample in one channel. Remaining channels can be used for other purposes, for example, a remaining channel can be used to measure background light or light variation across a sensor. This second channel measurement can be used for correction of the first sample collection channel. A third channel can be used for further corrections.

The device can be a commercially available cell phone with a cell phone camera. For example the device can be an iPhone.

The digital camera can have an image sensor made up of a plurality of pixels. For instance, the camera can have an image sensor with more than 1, 2, 3, 4, 5, 6, 7, 8, 10, 12, 14, 16, 18, 20, 22, 26, 30, 34, 38, 40, 44, 48, 52, 56, 60, 70, 80, 90, or 100 megapixels, for example. For instance, the camera can produce an image with more than 1, 2, 3, 4, 5, 6, 7, 8, 10, 12, 14, 16, 18, 20, 22, 26, 30, 34, 38, 40, 44, 48, 52, 56, 60, 70, 80, 90, or 100 megapixels, for example. In some embodiments, the camera can have an image sensor from about 6 megapixels to about 20 megapixels. In some embodiments, the camera can use a 41-megapixel sensor. The camera can use a 41-megapixel sensor with a pixel size of 1.4 μm.

In some embodiments the sensor is capable of being moved relative to sample. The image sensor may correct for movement of using software.

In some embodiments, the camera is a video camera. A video camera captures a plurality of images over time. In some embodiments, the video camera captures a plurality of images over time, and a subset of images are determined to be useful for further analysis. In some embodiments, a video camera captures a plurality of images, and a single image is selected for further analysis. The selection can be made by the user. The selection can be automated. The automated selection can be done by analysis of the contents of the image.

The image sensor can comprise one more lenses. The lens can be a lens typically found on a consumer digital camera or cell phone camera. For example a Carl Zeiss F2.4 8.02 mm lens. In some instances a second lens can be used.

The focal distances of a lens associate with an image sensor can be less than 100 cm, less than 90 cm, less than 80 cm, less than 70 cm, less than 60 cm, less than 50 cm, less than 40 cm, less than 30 cm, less than 20 cm, less than 10 cm, less than 5 cm, or less than 1 cm. For example a 0.67× magnetically mounted wide lens can be used. Using this objective images can be obtained, which auto-focus on the sample, at distances of 6.5 cm.

An image sensor can have an offset between a light source and a detector.

For example the image sensor can be the Nokia 808 PureView's 1/1.4″ CMOS sensor with a 41 MP resolution, outputting a maximum of 38 MP (at 4:3 aspect ratio); pixel size is 1.4 μm.

The image sensor can be a consumer digital camera or phone, for example a Nokia Pureview 808 cell phone. The image sensor can be a consumer digital portable computer or tablet. The image sensor can be a video camera. The image sensor can be included in a device such as a wristwatch. The image sensor can be an iPhone, Samsung Galaxy, or GoPro, for example.

Oversampling: for example images captured in the PureView modes are created by oversampling from the sensor's full resolution. Pixel oversampling bins many pixels to create a much larger effective pixel, thus increasing the total sensitivity of the pixel.

In some embodiments, a fluorescent dye is included in the assay. The fluorescent dye can be activated in the presence of nucleic acids. In some embodiments, the fluorescent dye is quenched in the presence of nucleic acid. Fluorescence is detected using an illumination source which provides excitation light at a wavelength absorbed by the fluorescent molecule, and a detection unit. The detection unit comprises a photosensor (such as a photomultiplier tube or charge-coupled device (CCD) array) to detect the emitted signal, and a mechanism (such as a wavelength-selective filter) to prevent the excitation light from being included in the photosensor output. The fluorescent molecules emit Stokes-shifted light in response to the excitation light, and this emitted light is collected by the detection unit. Stokes shift is the frequency difference or wavelength difference between emitted light and absorbed excitation light. A fluorescent dye can be any dye that is used in amplification reactions. A fluorescent dye can be a dye that binds single stranded DNA. A fluorescent dye can be a dye that binds double stranded DNA. A fluorescent dye can bind DNA or RNA. A fluorescent dye can be an intercalating dye. Some non-limiting examples of fluorescent dyes include, acridine dyes, cyanine dyes, fluorine dyes, oxazin dyes, phenanthridine dyes, rhodamine dyes, SYTO9, calcein, SYTO-13, SYTO-16, SYTO-64, SYTO-82, YO-PRO-1, SYTO-60, SYTO-62, SYTOX Orange, SYBR Green I, and TO-PRO-3, TaqMan dyes, Ethidium bromide, and EvaGreen, for example.

In some embodiments, the sample signal can be colormetric. The sample can change colors upon the amplification of a nucleic acid, for example. In some cases, a portion of the reaction medium can change colormetric properties that are sensed by the image sensor. The change of colormetric properties can be when a portion of the sample changes color in the presence of a specific or non-specific nucleic acid sequence. A change in colormetric properties can be a change in proportions of multiple colors. A change in colormetric properties can be a change in intensity of a color. In some embodiments, a colormetric signal can be detected when a portion of the reaction medium changes from clear to colored. In some embodiments, a colormetric signal can be detected when a portion of the reaction medium changes from one color to another. A color can be red, blue, green, purple, yellow, orange, indigo, violet, etc. A color of an object can be the set of wavelengths of visible light that are absorbed, reflected, and emitted by the object, for example. Additionally, colormetric signal can be the change of intensity of a color. A colormetric signal can be detected when a portion of the reaction medium changes from transparent to opaque or from opaque to transparent in the presence of a nucleic acid sequence, for example.

Reflected photons can be detected in some embodiments. Emitted photons can be detected in some embodiments. In some embodiments a combination of reflected and emitted photons are detected.

Multiplexed signal detection ensure that in multiplexed signal detection there is the ability to distinguish the amplification of many signals within the same volume as well as the ability to distinguish different signals from different volumes.

Electrochemiluminescence (ECL) emission is detected using a photosensor which is sensitive to the emission wavelength of the ECL species being employed. For example, transition metal-ligand complexes emit light at visible wavelengths, so conventional photodiodes and CCDs are employed as photosensors. An advantage of ECL is that, if ambient light is excluded, the ECL emission can be the only light present in the detection system, which improves sensitivity.

In some embodiments an electrochemiluminescence-based assay target detection obviates or reduces the need for an excitation light source, excitation optics, and/or optical filter elements, in turn, providing for a more compact and more inexpensive assay system. The absence of the requirement for the rejection of any excitation light also simplifies the detector circuitry, making the system even more inexpensive.

Nucleic acids can be detected from a sample. For example a cell phone camera can be used, in some embodiments, to detect nucleic acids of interested in a sample that had been loaded and on a SlipChip device.

The terms “nucleic acid” and “nucleic acid molecule” as used interchangeably herein, refer to a molecule comprised of nucleotides, i.e., ribonucleotides, deoxyribonucleotides, or both. The term includes monomers and polymers of ribonucleotides and deoxyribonucleotides, with the ribonucleotide and/or deoxyribonucleotides being connected together, in the case of the polymers, via 5′ to 3′ linkages. However, linkages may include any of the linkages known in the nucleic acid synthesis art including, for example, nucleic acids comprising 5′ to 2′ linkages. The nucleotides used in the nucleic acid molecule may be naturally occurring or may be synthetically produced analogues that are capable of forming base-pair relationships with naturally occurring base pairs. Examples of non-naturally occurring bases that are capable of forming base-pairing relationships include, but are not limited to, aza and deaza pyrimidine analogues, aza and deaza purine analogues, and other heterocyclic base analogues, wherein one or more of the carbon and nitrogen atoms of the purine and pyrimidine rings have been substituted by heteroatoms, e.g., oxygen, sulfur, selenium, phosphorus, and the like.

The term “oligonucleotide” as used herein refers to a nucleic acid molecule comprising multiple nucleotides. An oligonucleotide can comprise about 2 to about 300 nucleotides.

The term “modified oligonucleotide” as used herein refer to oligonucleotides with one or more chemical modifications at the molecular level of the natural molecular structures of all or any of the bases, sugar moieties, internucleoside phosphate linkages, as well as molecules having added substituents, such as diamines, cholesterol or other lipophilic groups, or a combination of modifications at these sites. The internucleoside phosphate linkages can be phosphodiester, phosphotriester, phosphoramidate, siloxane, carbonate, carboxymethylester, acetamidate, carbamate, thioether, bridged phosphoramidate, bridged methylene phosphonate, phosphorothioate, methylphosphonate; phosphorodithioate, bridged phosphorothioate and/or sulfone internucleotide linkages, or 3′-3′, 5′-2′ or 5′-5′ linkages, and combinations of such similar linkages (to produce mixed backbone modified oligonucleotides). The modifications can be internal (single or repeated) or at the end(s) of the oligonucleotide molecule and can include additions to the molecule of the internucleoside phosphate linkages, such as cholesteryl, diamine compounds with varying numbers of carbon residues between amino groups and terminal ribose, deoxyribose and phosphate modifications which cleave or cross-link to the opposite chains or to associated enzymes or other proteins. Electrophilic groups such as ribose-dialdehyde could covalently link with an epsilon amino group of the lysyl-residue of such a protein. A nucleophilic group such as n-ethylmaleimide tethered to an oligomer could covalently attach to the 5′ end of an mRNA or to another electrophilic site. The term “modified oligonucleotides” also includes oligonucleotides comprising modifications to the sugar moieties such as 2′-substituted ribonucleotides, or deoxyribonucleotide monomers, any of which are connected together via 5′ to 3′ linkages. Modified oligonucleotides may also be comprised of PNA or morpholino modified backbones where target specificity of the sequence is maintained. A modified oligonucleotide of the invention (1) does not have the structure of a naturally occurring oligonucleotide and (2) will hybridize to a natural oligonucleotide. Further, the modification preferably provides (3) higher binding affinity, (4) greater acid resistance, and (5) better stability against digestion with enzymes as compared to a natural oligonucleotide.

The term “oligonucleotide backbone” as used herein refers to the structure of the chemical moiety linking nucleotides in a molecule. The invention preferably comprises a backbone which is different from a naturally occurring backbone and is further characterized by holding bases in correct sequential order and (2) holding bases a correct distance between each other to allow a natural oligonucleotide to hybridize to it. This may include structures formed from any and all means of chemically linking nucleotides. A modified backbone as used herein includes modifications (relative to natural linkages) to the chemical linkage between nucleotides, as well as other modifications that may be used to enhance stability and affinity, such as modifications to the sugar structure. For example an a-anomer of deoxyribose may be used, where the base is inverted with respect to the natural b-anomer. In a preferred embodiment, the 2′-OH of the sugar group may be altered to 2′-O-alkyl or 2′-O-alkyl-n(O-alkyl), which provides resistance to degradation without comprising affinity.

The nucleic acids can be extracted before analysis. The exact protocol used to extract nucleic acids depends on the sample and the exact assay to be performed. For example, the protocol for extracting viral RNA will vary considerably from the protocol to extract genomic DNA. However, extracting nucleic acids from target cells usually involves a cell lysis step followed by nucleic acid purification. The cell lysis step disrupts the cell and nuclear membranes, releasing the genetic material. This is often accomplished using a lysis detergent, such as sodium dodecyl sulfate, which also denatures the large amount of proteins present in the cells.

The nucleic acids are then purified with an alcohol precipitation step, usually ice-cold ethanol or isopropanol, or via a solid phase purification step, typically on a silica matrix in a column, resin or on paramagnetic beads in the presence of high concentrations of a chaotropic salt, prior to washing and then elution in a low ionic strength buffer. An optional step prior to nucleic acid precipitation is the addition of a protease which digests the proteins in order to further purify the sample.

Other lysis methods include mechanical lysis via ultrasonic vibration and thermal lysis where the sample is heated to 94° C. to disrupt cell membranes.

The target DNA or RNA may be present in the extracted material in very small amounts, particularly if the target is of pathogenic origin. Nucleic acid amplification provides the ability to selectively amplify (that is, replicate) specific targets present in low concentrations to detectable levels.

In some embodiments, the assay is an amplification reaction assay. In some embodiments a cell phone camera is used to detect a amplified nucleic acid on a SlipChip device.

The most commonly used nucleic acid amplification technique is the polymerase chain reaction (PCR). The amplification reaction assay can be PCR. PCR is well known in this field and comprehensive description of this type of reaction is provided in E. van Pelt-Verkuil et al., Principles and Technical Aspects of PCR Amplification, Springer, 2008.

PCR is a powerful technique that amplifies a target DNA sequence against a background of complex DNA. If RNA is to be amplified (by PCR), it must be first transcribed into cDNA (complementary DNA) using an enzyme called reverse transcriptase. Afterwards, the resulting cDNA is amplified by PCR.

PCR is an exponential process that proceeds as long as the conditions for sustaining the reaction are acceptable. The components of the reaction are:

1. pair of primers—short single strands of DNA with around 10-30 nucleotides complementary to the regions flanking the target sequence
2. DNA polymerase—a thermostable enzyme that synthesizes DNA
3. deoxyribonucleoside triphosphates (dNTPs)—provide the nucleotides that are incorporated into the newly synthesized DNA strand
4. buffer—to provide the optimal chemical environment for DNA synthesis.

In embodiments using PCR, the components of the reaction can be in contact with sample. The components of the reaction can be added to a container that holds the sample. The components of the reaction can be present in a container, and the sample can be added. In some embodiments, a kit can comprise a plurality of small containers, at least one container holding the components of a PCR reaction. A kit can comprise a SlipChip and the components of the reaction.

PCR typically involves placing these reactants in a small tube (^{˜}10-50 microlitres) containing the extracted nucleic acids. The tube is placed in a thermal cycler; an instrument that subjects the reaction to a series of different temperatures for varying amounts of time. The standard protocol for each thermal cycle involves a denaturation phase, an annealing phase, and an extension phase. The extension phase is sometimes referred to as the primer extension phase. In addition to such three-step protocols, two-step thermal protocols can be employed, in which the annealing and extension phases are combined. The denaturation phase typically involves raising the temperature of the reaction to 90-95° C. to denature the DNA strands; in the annealing phase, the temperature is lowered to ^{˜}50-60° C. for the primers to anneal; and then in the extension phase the temperature is raised to the optimal DNA polymerase activity temperature of 60-72° C. for primer extension. This process is repeated cyclically around 20-40 times, the end result being the creation of millions of copies of the target sequence between the primers.

The amplification reaction assay can be a variant of PCR. The amplification reaction assay can be selected from the group of variants to the standard PCR protocol such as multiplex PCR, linker-primed PCR, direct PCR, tandem PCR, real-time PCR and reverse-transcriptase PCR, amongst others, which have been developed for molecular diagnostics.

The amplification reaction assay can be multiplex PCR. Multiplex PCR uses multiple primer sets within a single PCR mixture to produce amplicons of varying sizes that are specific to different DNA sequences. By targeting multiple genes at once, additional information may be gained from a single test-run that otherwise would require several experiments.

In some embodiments, a multiplexed PCR reaction is performed where a plurality of primer sets are added to a reaction mixture and each amplify their specified target within the same volume, for example. In other embodiments a sample is split into a plurality of smaller volumes into which single primer sets are introduced.

The amplification reaction assay can be linker-primed PCR, also known as ligation adaptor PCR. Linker-primed PCR is a method used to enable nucleic acid amplification of essentially all DNA sequences in a complex DNA mixture without the need for target-specific primers. The method firstly involves digesting the target DNA population with a suitable restriction endonuclease (enzyme). Double-stranded oligonucleotide linkers (also called adaptors) with a suitable overhanging end are then ligated to the ends of target DNA fragments using a ligase enzyme. Nucleic acid amplification is subsequently performed using oligonucleotide primers which are specific for the linker sequences. In this way, all fragments of the DNA source which are flanked by linker oligonucleotides can be amplified.

The amplification reaction assay can be direct PCR. Direct PCR describes a system whereby PCR is performed directly on a sample without any, or with minimal, nucleic acid extraction. With appropriate chemistry and sample concentration it is possible to perform PCR with minimal DNA purification, or direct PCR. Adjustments to the PCR chemistry for direct PCR include increased buffer strength, the use of polymerases which have high activity and processivity, and additives which chelate with potential polymerase inhibitors.

The amplification reaction assay can be tandem PCR. Tandem PCR utilizes two distinct rounds of nucleic acid amplification to increase the probability that the correct amplicon is amplified. One form of tandem PCR is nested PCR in which two pairs of PCR primers are used to amplify a single locus in separate rounds of nucleic acid amplification. The amplification reaction assay can be nested PCR. The first pair of primers hybridize to the nucleic acid sequence at regions external to the target nucleic acid sequence. The second pair of primers (nested primers) used in the second round of amplification bind within the first PCR product and produce a second PCR product containing the target nucleic acid, that can be shorter than the first one. The logic behind this strategy is that if the wrong locus were amplified by mistake during the first round of nucleic acid amplification, the probability is very low that it would also be amplified a second time by a second pair of primers and thus increases specificity.

The amplification reaction assay can be real-time PCR. The amplification reaction assay can be quantitative PCR. Real-time PCR, or quantitative PCR, is used to measure the quantity of a PCR product in real time. By using a fluorophore-containing probe or fluorescent dyes along with a set of standards in the reaction, it is possible to quantify the starting amount of nucleic acid in the sample. This is particularly useful in molecular diagnostics where treatment options may differ depending on the pathogen load in the sample.

The amplification reaction assay can be reverse-transcriptase PCR (RT-PCR). Reverse-transcriptase PCR (RT-PCR) is used to amplify DNA from RNA. Reverse transcriptase is an enzyme that reverse transcribes RNA into complementary DNA (cDNA), which is then amplified by PCR. RT-PCR can be used in expression profiling, to determine the expression of a gene or to identify the sequence of an RNA transcript, including transcription start and termination sites. It can be used to amplify RNA viruses such as human immunodeficiency virus or hepatitis C virus.

The amplification reaction assay can be isothermal. Isothermal amplification is another form of nucleic acid amplification which does not rely on the thermal denaturation of the target DNA during the amplification reaction and hence does not require sophisticated machinery. Isothermal nucleic acid amplification methods can therefore be carried out in primitive sites or operated easily outside of a laboratory environment. A non-limiting list of isothermal nucleic acid amplification methods is Strand Displacement Amplification, Transcription Mediated Amplification, Nucleic Acid Sequence Based Amplification, Recombinase Polymerase Amplification, Rolling Circle Amplification, Ramification Amplification, Helicase-Dependent Isothermal DNA Amplification and Loop-Mediated Isothermal Amplification, for example.

Isothermal nucleic acid amplification methods, can rely on alternative methods such as enzymatic nicking of DNA molecules by specific restriction endonucleases, the use of an enzyme to separate the DNA strands at a constant temperature, or single stranded segments which are generated during the amplification, for example.

The amplification reaction assay can be Strand Displacement Amplification (SDA). Strand Displacement Amplification (SDA) can rely on the ability of certain restriction enzymes to nick the unmodified strand of hemi-modified DNA and the ability of a 5′-3′ exonuclease-deficient polymerase to extend and displace the downstream strand. Exponential nucleic acid amplification can then achieved by coupling sense and antisense reactions in which strand displacement from the sense reaction serves as a template for the antisense reaction. The use of nickase enzymes which do not cut DNA in the traditional manner but produce a nick on one of the DNA strands, such as N. Alw1, N. BstNB1 and Mly1, for example, can be used in this reaction. SDA has been improved by the use of a combination of a heat-stable restriction enzyme (Ava1) and heat-stable Exo-polymerase (Bst polymerase). This combination has been shown to increase amplification efficiency of the reaction from 108 fold amplification to 1010 fold amplification so that it is possible using this technique to amplify unique single copy molecules.

The amplification reaction assay can be Transcription Mediated Amplification (TMA). The amplification reaction assay can be Nucleic Acid Sequence Based Amplification (NASBA). Transcription Mediated Amplification (TMA) and Nucleic Acid Sequence Based Amplification (NASBA) can use an RNA polymerase to copy RNA sequences but not corresponding genomic DNA. The technology can use two primers and two or three enzymes, RNA polymerase, reverse transcriptase and optionally RNase H (if the reverse transcriptase does not have RNase activity). One primer can contain a promoter sequence for RNA polymerase. In the first step of nucleic acid amplification, this primer hybridizes to the target ribosomal RNA (rRNA) at a defined site. Reverse transcriptase can create a DNA copy of the target rRNA by extension from the 3′ end of the promoter primer. The RNA in the resulting RNA:DNA duplex can be degraded by the RNase activity of the reverse transcriptase if present or the additional RNase H. Next, a second primer binds to the DNA copy. A new strand of DNA is synthesized from the end of this primer by reverse transcriptase, creating a double-stranded DNA molecule. RNA polymerase recognizes the promoter sequence in the DNA template and initiates transcription. Each of the newly synthesized RNA amplicons re-enters the process and serves as a template for a new round of replication.

The amplification reaction assay can be Recombinase Polymerase Amplification (RPA). In Recombinase Polymerase Amplification (RPA), the isothermal amplification of specific DNA fragments is achieved by the binding of opposing oligonucleotide primers to template DNA and their extension by a DNA polymerase. Heat is not always required to denature the double-stranded DNA (dsDNA) template. Instead, RPA can employ recombinase-primer complexes to scan dsDNA and facilitate strand exchange at cognate sites. The resulting structures are stabilized by single-stranded DNA binding proteins interacting with the displaced template strand, thus preventing the ejection of the primer by branch migration. Recombinase disassembly leaves the 3′ end of the oligonucleotide accessible to a strand displacing DNA polymerase, such as the large fragment of Bacillus subtilis Pol I (Bsu), and primer extension ensues. Exponential nucleic acid amplification is accomplished by the cyclic repetition of this process.

The amplification reaction assay can be Helicase-dependent amplification (HDA). Helicase-dependent amplification (HDA) mimics the in vivo system in that it uses a DNA helicase enzyme to generate single-stranded templates for primer hybridization and subsequent primer extension by a DNA polymerase. In the first step of the HDA reaction, the helicase enzyme traverses along the target DNA, disrupting the hydrogen bonds linking the two strands which are then bound by single-stranded binding proteins. Exposure of the single-stranded target region by the helicase allows primers to anneal. The DNA polymerase then extends the 3′ ends of each primer using free deoxyribonucleoside triphosphates (dNTPs) to produce two DNA replicates. The two replicated dsDNA strands independently enter the next cycle of HDA, resulting in exponential nucleic acid amplification of the target sequence.

The amplification reaction assay can be Rolling Circle Amplification (RCA). Other DNA-based isothermal techniques include Rolling Circle Amplification (RCA) in which a DNA polymerase extends a primer continuously around a circular DNA template, generating a long DNA product that consists of many repeated copies of the circle. By the end of the reaction, the polymerase generates many thousands of copies of the circular template, with the chain of copies tethered to the original target DNA. This allows for spatial resolution of target and rapid nucleic acid amplification of the signal. Up to 1012 copies of template can be generated in 1 hour. Ramification amplification is a variation of RCA and utilizes a closed circular probe (C-probe) or padlock probe and a DNA polymerase with a high processivity to exponentially amplify the C-probe under isothermal conditions.

The amplification reaction assay can be Loop-mediated isothermal amplification (LAMP). LAMP offers high selectivity and employs a DNA polymerase and a set of four specially designed primers that recognize a total of six distinct sequences on the target DNA. An inner primer containing sequences of the sense and antisense strands of the target DNA initiates LAMP. The following strand displacement DNA synthesis primed by an outer primer releases a single-stranded DNA. This serves as template for DNA synthesis primed by the second inner and outer primers that hybridize to the other end of the target, which produces a stem-loop DNA structure. In subsequent LAMP cycling one inner primer hybridizes to the loop on the product and initiates displacement DNA synthesis, yielding the original stem-loop DNA and a new stem-loop DNA with a stem twice as long. The cycling reaction continues with accumulation of many copies of target in less than an hour. The final products are stem-loop DNAs with several inverted repeats of the target and cauliflower-like structures with multiple loops formed by annealing between alternately inverted repeats of the target in the same strand.

In some embodiments, the amplification is a one step digital reverse-transcription loop-mediated isothermal amplification (dRT-LAMP) reaction for quantifying HIV-1 viral load with all reactions performed. LAMP produces a bright fluorescence signal through replacement of manganese with magnesium in calcein. In some embodiments, this fluorescence can then be detected and counted using a commercial cell phone camera.

In some embodiments, the amplification is a two-step dRT-LAMP reaction for quantifying HIV-1 viral load. The two-step dRT-LAMP decouples the reverse transcription step and the subsequent amplification step. During the reverse transcription step, a single-stranded DNA template or cDNA is synthesized from RNA. During the amplification step, LAMP reagent mixture and the remaining primers are added and amplification of the cDNA occurs. In some embodiments, Backward loop primer (BlP) is incorporated into the first step. The rate of strand displacement synthesis (e.g. release of cDNA from the RNA:cDNA hybrid) can interfere with amplification during the second step. In some embodiments, RNase H is incorporated into the second step to break up the hybrid and improve efficiency.

The amplified product can be analyzed to determine whether the anticipated amplicon (the amplified quantity of target nucleic acids) was generated. Single molecule counting using dLAMP and dRT-LAMP is attractive because it is isothermal and therefore does not require thermocycling equipment, is compatible with plastics, and provides a bright signal from the calcein detection system which should be readable by a cell phone. In some embodiments, the present invention provides a platform for a multi-step manipulation utilizing dRT-LAMP. In some embodiments, the present invention can be applied to technologies that enable multistep manipulation of many volumes in parallel, e.g. for mechanistic studies of dLAMP and other digital, single-molecule reactions. In some embodiments, the present invention can be applicable under resource-limited settings (RLS) for deploying digital single molecule amplification for diagnostics applications.

In some embodiments the amplification employed may take place in a variety of different mediums, such as for example, aqueous solution, polymeric matrix, solid support, etc.

A fluorescent region can correspond to an amplification product from a single molecule. In some embodiments multiple single molecule signals are detected and resolved in the same image. The fluorescent region can be detected. Single-molecule analysis can, in some embodiments, provide better sensitivity and a simpler method for quantification. One way single-molecule analysis can be performed is through fluorescent labeling and detection of individual molecules. Historically, counting these molecules has been a tedious procedure and it must be performed on an expensive microscope at very high magnification with a small field of view, leading to the need to raster through the sample.

The processes herein can be called binary quantification. The processes herein can be called binary analyses. The process of binary quantification begins with a sample that may contain an analyte. The analyte can be a molecule to be quantified or searched for, for instance a particular nucleic acid, a particular nucleic acid sequence, a gene, or a protein, for example. The sample can be partitioned into many separate reaction volumes. In some embodiments, the reaction volumes are separate analysis regions. In some embodiments, the separate reaction volumes are physically separated in separate wells, chambers, areas on the surface of a slide, droplets, beads, or aliquots, for example. In some embodiments, the separate reaction volumes can be in the same container, for instance, the analyte can be affixed to a substrate or attached to a bead. The reaction volumes can be on beads, on the surface of a slide, or attached to a substrate. The sample is distributed to many separate reaction volumes such that each individual reaction volume contains either zero individual molecules of the analyte, or one or more individual molecules of the analyte. One or more molecules can mean a non-zero number of molecules. One or more molecules can mean one molecule. In some embodiments, one or more molecules can mean one molecule, two molecules, three molecules, four molecules . . . etc. In some embodiments, each separate reaction volume is contained in a well. In some embodiments, the sample is distributed such that each reaction volume, on average comprises less than one individual molecule of the analyte. In some embodiments, the sample is distributed such that most reaction volumes comprise either zero or one molecules of the analyte. Next, a qualitative “yes or no” test can be done to determine whether or not each reaction volume contains one or more analyte molecules by reading the pattern of discrete positive and negative reaction volumes. A positive reaction volume can be a reaction volume determined to contain one or more analyte molecules. A positive reaction volume can be a reaction volume determined to have a signal that correlates to the presence of one or more analyte molecules. A positive reaction volume can be a reaction volume determined to have a signal above a threshold that correlates to the presence of one or more analyte molecules. In some embodiments, a positive reaction volume is quantified as 1, or a simple multiple of 1 such as 2, 3, etc. while a negative reaction volume is quantified as 0. In some embodiments, a positive reaction volume is quantified as 1 and a negative reaction volume is quantified as 0. A negative reaction volume can be a reaction volume determined to contain zero analyte molecules. A negative reaction volume can be a reaction volume that does not have a signal that correlates to the presence of one or more analyte molecules. A negative reaction volume can be a reaction volume that does not have a signal above the threshold that correlates to the presence of one or more analyte molecules. The determination and/or designation of each reaction volume as a positive or a negative reaction volume can be referred to as a binary assay or a digital assay. This “yes or no test” or test like this can be referred to as a binary assay. This qualitative analysis of which reaction volume are negative reaction volume and which reaction volume are positive reaction volume can then be translated into a quantitative concentration of analyte in the sample using Poisson analysis. A high dynamic range can be achieved through using many reaction volumes. A high dynamic range can be achieved by using a device that has reaction volume of different sizes. A high dynamic range can be achieved by partitioning the sample into many wells and/or into wells of different sizes. This overall process can be called binary quantification of nucleic acids. This process can be called counting molecules of analyte. In some embodiments, binary quantification is the process of partitioning a sample into a plurality of reaction volume such that each reaction volume contains either zero or a non-zero number of analyte molecules; determining and/or designating which reaction volume are positive reaction volume and which reaction volume are negative reaction volume with respect to the analyte molecule; and translating the information about positive and negative reaction volume into information about the quantity or concentration of the analyte molecule in the sample. In some embodiments, the absolute number of analyte molecules is determined. In some embodiments, the translation of the information about which reaction volume are positive reaction volume and which reaction volume are negative reaction volume to information about the amount, absolute number of molecules, or concentration of the analyte in the sample is called digital quantification of the analyte. In some embodiments, the analyte is a nucleic acid. In some embodiments, the binary quantification of nucleic acids is achieved. In some embodiments, binary quantification of a nucleic acid analyte is determined wherein the sample is partitioned into several reaction volumes, wherein the reaction volumes are on a SlipChip.

In some embodiments, a binary quantification of analyte molecules in a sample can be achieved without spatially separating the sample into multiple reaction volumes. In these embodiments, the analyte molecules can be counted by informational separation. In some embodiments, analyte molecules in the sample undergo a binary quantification through a process wherein the analyte molecules are tagged with a pool of information-carrying molecules, amplified or copied, and the number of distinct information-carrying molecules that were amplified or copied is counted in to get a quantification of the starting number of analyte molecules (see e.g. WO 2012148477). In some embodiments, the information-carrying molecule can be a pool of chemical barcodes. In some embodiments, the information-carrying molecule can be a set of nucleic acid sequences.

Digital analyses can be achieved using the polymerase chain reaction (PCR), recombinant polymerase amplification (RPA), and loop mediated amplification (LAMP) as a way of quantifying RNA or DNA concentrations. Amplifications such as RPA and LAMP, which can use isothermal chemistries, can be well suited for home and limited-resource setting use. LAMP chemistry in particular is an attractive candidate for use in a home or limited-resource setting platform as it can have a relatively broad temperature tolerance range, can work with simple and cheap chemical-based heaters and phase-change materials, and can have a fluorescence gain with positive wells.

Described herein, in certain embodiments, are a device for and methods of analyzing fluorescent patterns using a mobile communication device, and transmitting and processing information. Such capability is valuable for many purposes, including the analysis of digital nucleic acid amplification reactions.

### Robustness

Robustness can be the degree to which a series of repeated quantitative measurements provides a set of similar measurements under varying experimental conditions. For example a cell phone camera may be used to successfully perform similar measurements on a SlipChip under a variety of conditions found in the real world. Similar measurements can be identical measurements. Similar measurements can be the same diagnosis. Similar measurements can be the same answer. Similar measurements can mean more than one measurement within experimental error of each other. Similar measurements can yield a consistent outcome with statistical significance. Similar measurements can be of similar numerical size, for instance within 5%, 10%, 15%, 20%, 25%, 30%, 40%, 50%, 60%, 70%, 80%, 90%, 100%, 200%, 1,000% of each other. Robust assays can produce similar measurements more often than 25%, 30%, 40%, 50%, 60%, 70%, 80%, 85%, 90%, 95%, 96%, 97%, 98%, 99%, 99.5%, 99.9%, 99.99%, for example, of instances measured under a given set of conditions.

Different types of assays can be robust assays. A nucleic acid amplification and quantification assay can be robust. An assay to detect a protein or other target such as a cell, exosome, liposome, bacteria, virus, etc. can be robust. A LAMP assay can be robust. A RT-LAMP assay can be robust. A dRT-LAMP assay can be robust. A binary LAMP reaction can be robust. A binary, two-step LAMP reaction can be robust. A PCR reaction can be robust. A qPCR assay can be robust. A quantitative nucleic acid amplification reaction can be robust. A qualitative nucleic acid amplification reaction can be robust. A method to diagnosis a health outcome based on the amplification of a nucleic acid sequence can be robust. A process within a SlipChip can be robust. The imaging and analysis of a SlipChip after a LAMP reaction can be a robust process.

The absolute efficiency of dRT-LAMP can be increased over 10-fold, e.g. from ˜2% to ˜28%, by i) using a more efficient reverse transcriptase, ii) introducing RNase H to break up the DNA-RNA hybrid, and iii) adding only the BIP primer during the RT step. dRT-LAMP can be compatable with a plastic SlipChip device and used this two-step method to quantify HIV RNA. The dRT-LAMP quantification results were in some cases very sensitive to the sequence of the patient's HIV RNA.

Assays can be robust with respect to experimental variables. An assay can be robust with respect to a given temperature range. An assay can be robust of over a temperature range. Some non-limiting ranges, over which an assay can be robust include 1° C., 2° C., 3° C., 4° C., 5° C., 6° C., 7° C., 8° C., 9° C., 10° C., 11° C., 12° C., 16° C., 20° C., 24° C., 28° C., 32° C., 40° C., 50° C., 60° C., 80° C., 100° C., 150° C., 200° C., 250° C., or 300° C., for example. The temperature range of which an assay is robust can be centered on temperature on an absolute temperature scale. Some non-limiting temperatures that could be the center of the temperature range that an assay is robust to include −40° C., −30° C., −20° C., −10° C., 0° C., 10° C., 20° C., room temperature, 25° C., 30° C., 35° C., body temperature, 37° C., 40° C., 45° C., 50° C., 55° C., 60° C., 65° C., 70° C., 80° C., 90° C., 100° C., 110° C., 150° C., or 200° C., for example. In some embodiments, a binary LAMP assay is used to amplify and subsequently image and quantify a nucleic acid sequence in a sample. In these embodiments, the assay can be a robust quantification of a nucleic acid sequence with over a temperature range of 9° C. centered at about 60° C. A binary LAMP assay used to amplify and subsequently image and quantify a nucleic acid sequence in a sample can be robust over the temperature range from about 55° C. to about 66° C. In some embodiments, a SlipChip can be imaged and the data can be processed to give robust findings over a range of a temperature from about 5° C. to about 70° C.

An assay can be robust with respect to time. An assay can give consistent results over a range of time points. An assay can require only end-point readout. A binary DNA amplification experiment can require only end-point readout. The endpoint read out can be obtained near the completion of amplification, or at a time after this time point. A robust DNA amplification assay can give consistent results at a time point near the end of the reaction and/or at a timepoint after the reaction is complete. A non-limiting range of reaction time that an assay could be robust over includes 0.01 min, 0.1 min, 0.5 min, 1 min, 2 min, 3 min, 4 min, 5 min, 6 min, 7 min, 8 min, 9 min, 10 min, 12 min, 14 min, 16 min, 20 min, 24 min, 28 min, 32 min, 40 min, 45 min, 50 min, 1.0 hour, 2 hour, 3 hour, 4 hour, 5 hour, 6 hour, 8 hour, 10 hour, 12 hour, 16 hour, 18 hour, 1 day, 2 day, 3 day, 7 days, 1 month, or 1 year, for example. In some cases, binary DNA amplification experiments do not require exact knowledge of time. The output of a binary DNA amplification can be robust to variation in reaction time beyond the optimal reaction time. In some embodiments, a d-LAMP assay on a SlipChip is robust over a 20 minute time period between 40 minutes and 60 minutes after the LAMP reaction begins, for example.

An assay can be robust with respect to variations in atmospheric humidity. In some embodiments, an assay can be robust regardless of the atmospheric humidity. In some embodiments, an assay can be robust over a range of atmospheric humidity. The range of humidity can be from about 0% to 100% relative humidity. The range of atmospheric humidity at which an assay can be robust can be from about 0 to about 40 grams water per cubic meter of air at about 30° C. In some embodiments, an assay can be robust from about 0% humidity to about 40%, 50%, 60%, 70%, 80%, 90%, or 100% humidity, for example. In some embodiments, an assay can be robust over a humidity range of about 40%, 50%, 60%, 70%, 80%, 90%, or 100% humidity. In some embodiments, a d-LAMP assay run in a SlipChip can be imaged and analyzed as a robust assay over a range of humidity from about 0% to about 100% atmospheric humidity.

An assay can be robust with respect to equipment used to perform the experiment. For example, an assay can be robust with respect to the type of camera used. An assay can be robust with respect to the number of pixels in the image recorded by the camera. An assay can be robust with respect to the software system running on the device that captures the data. An assay can be robust with respect to the sample container. An assay can be robust with respect to using a cellphone with a built in camera versus using specialized equipment. An assay can be robust with respect to the type of camera flash present on the camera device used. An assay can be robust with respect to having imaging performed with non-quantitative consumer electronic devices such as cell phones, tablets, or small handheld computers. An assay can be robust with respect to an external excitation light source.

An assay can be robust with respect to camera flash inconsistency. An assay can be robust with respect to mechanism of flash. For example, an assay could yield robust and consistent result with a Xenon flash or an LED flash. An assay can be robust with respect to flash size. An assay can be robust with respect to flash direction. An assay can be robust with respect to the flash direction. In some embodiments, the direction the flash is pointed can yield consistent results. In some embodiments, the timing of the flash can be inconsistent, and the assay can be robust over a range of potential flash timings.

An assay can be robust with respect to external light source inconsistency. An assay can be robust with respect to the orientation of an external light source. An assay can be robust with respect to the type of light source used to generate the signal, such as, for example, light emitting diodes, compact fluorescent lights, incandescent lights, xenon flashes, etc. An assay can be robust with respect to the external light source intensity. An assay can be robust with respect to the color of an external light source.

An assay can be robust with respect to variations in the amount of background light present during imaging. In some embodiments, whether conducted in a dark room or in the presence of background light, an assay can give consistent results. In some embodiments, a d-LAMP assay can be robust over a range of background lighting. Some non-limiting examples of ranges of background lighting that an assay can be robust over can be from about 0 lux, 0.1, 0.2, 0.5, 0.8, 1.0 to about 1, 2, 3, 4, 5, 6, 7, 8, 9, 10, 12, 14, 16, 20, 24, 28, 32, 36, 40, 50, 60, 70, 80, 90, 100, 200, 300, 400, 500, 600, 700, 800, 900, or 1000 lux, for example. An assay can be robust with respect to ambient daylight. In some embodiments, an assay can be robust whether in a dark room, or carried out with a cell phone placed in a shoe box.

In some embodiments, the assay provides a quantitative analytical measurement. For instance, the invention can measure and display the amount and/or the concentration of a nucleic acid sequence within a sample as a quantitative amount. This measurement can be robust with respect to the experimental conditions present during the chemical amplification of the nucleic acid sequence, during the measurement of the optical data, and/or during the processing of the data, for instance. Examples of experimental perturbations or varying experimental conditions include, but are not limited to, for example variation of temperature of several degrees Celsius., variations in atmospheric humidity, imaging performed with non-quantitative consumer electronic devices such as cell phones, variations in assay time, camera flash inconsistency, sampling errors, variations in the amount of background light present during imagining. In some embodiments, a binary LAMP assay is used to amplify and subsequently image and quantify a nucleic acid sequence in a sample. In these embodiments, an accurate and reproducible quantification of the sequence can be obtained with a variation of temperature from about 55° C. to about 66° C., over a time period of 15 min-1.5 hours, in the presence of 0-100% atmospheric humidity, when the measurement is obtained with a cell phone camera that is not confined to a dark room. An assay can be robust with respect to variation of multiple experimental variables within a single experiment. For example, a binary LAMP assay taking place in a SlipChip can be robust and yield consistent results over a range of reaction temperature, reaction time, and amount background light presence during imaging for a given sample. For example, a binary LAMP assay taking place in a SlipChip can be robust and yield similar results when data is obtained from imaging with a cellphone in a shoebox, with reaction time varying from 40 min, 50 min to 60 min, over a six-degree temperature range (temperature range 55-66° C.).

A sample can be contained or received by a sample container, e.g. a SlipChip. A SlipChip is a device that can hold the sample. A SlipChip holding a sample can be imaged. In some embodiments, a SlipChip is composed of two pieces of glass slides with complementary patterns were made with using standard photolithographic and wet chemical etching techniques. Soda-lime glass plates with chromium and photoresist coating were obtained from. Telic Company (Valencia, Calif.). The glass plate with photoresist coating was aligned with a photomask containing the design of the microducts and areas using a Karl Suss, MJBB3 contact alighner. The photomask may also contain marks to align the mask with the plate. The glass plate and photomask were then exposed to UV light for 1 min. The photomask was removed, and the glass plate was developed by immersing it in 0.1 mol/L NaOH solution for 2 min. Only the areas of the photoresist that were exposed to the UV light dissolved in the solution. The exposed underlying chromium layer was removed using a chromium etchant (a solution of 0.6:0.365 M HClO₄/(NH₄)₂Ce(N₃)₆). The plate was rinsed with Millipore water and dried with nitrogen gas, and the back of the glass plate was taped with PVC sealing tape (McMaster-Carr) to protect the back side of glass. The taped glass plate was then carefully immersed in a plastic container with a buffered etching agent composed of 1:0.5:0.75 mol/L. HF/NH₄F/HNO₃ to etch the soda-lime glass at the temperature of 40° C. The etching speed was controlled by the etching temperature, and the area and duct depth was controlled by the etching time. After etching, the tape was removed from the plates. The plate was then thoroughly rinsed with Millipore water and dried with nitrogen gas. The remaining photoresist was removed by rinsing with ethanol, and the remaining chromium coating was removed by immersing the plate in the chromium etchant. The surface of the glass plate were rendered hydrophobic by silanization with tridecafluoro-1,1,2,2-tetrahydrooctyl-1-trichlorosilane (United Chemical Technologies, Inc.), Access holes were drilled with a 0.76 mm diameter diamond drill bit.

One method to establish fluidic communication between two or more areas of the SlipChip includes the use of a channel with at least one cross-sectional dimension in the nanometer range, a nanochannel, which can be embedded in the SlipChip. The nanochannels can be embedded into multilayer SlipChip. The height of nanochannel can be varied with nanometer scale resolution. The height of the nanochannedl can prohibit transfer of micron sized cells between the wells, but enable transfer of proteins, vesicles, micelles, genetic material, small molecules, ions, and other molecules and macromolecules, including cell culture media and secreted products. The width, length, and tortuosity of the nanochannels can also be manipulated in order to control transport dynamics between wells. Nanochannels can be fabricated as described in Bacterial metapopulations nanofabricated landscapes, Juan E. Keymer, Peter Galajda, Muldoon, Sungsu Park, and Robert H. Austin, PNAS Nov. 14, 2006 vol. 103 no. 46 17290-17295, or by etching nanochannels in the first glass piece and bringing it in contact with the second glass piece, optionally followed by a bonding step. Applications include filtration, capturing of cells and particles, long term cell culture, and controlling interactions among cells and cellular colonies and tissues.

SlipChip devices of the PDMS/Glass type may also be made using soft lithography, similarly as described previously. The device used contains two layers, each layer was composed of a thin membrane of PDMS with ducts and areas, and a 1 mm thick microscope glass slides with size of 75 mm×25 min. To make the device, the glass slides were cleaned and subjected to an oxygen plasma treatment. Dow-Corning Sylgard 184 A and B components were mixed at a mass ratio of 5:1, and poured onto the mold of the SlipChip. A glass slide was placed onto the PDMS before cure. A glass bottom with iron beads were place onto the glass slides to make the PDMS membrane thinner. The device were pre-cured for 7 hour at room temperature, then move to 60° C. oven and cured overnight. After cure, the device were peeled off the mold and silanized with tridecafluoro-1,1,2,2-tetrahydrooctyl-1-trichlorosilane. Access holes were drilled with a 076 mm diameter diamond drill bit.

Polymeric materials suitable for use with the invention may be organic polymers. Such polymers may be homopolymers or copolymers, naturally occurring or synthetic, crosslinked or uncrosslinked. Specific polymers of interest include, hut are not limited to, polyimides, polycarbonates, polyesters, polyamides, polyethers, polyurethanes, polyfluorocarbons, polystyrenes, poly(acrylonitrile-butadiene-styrene)(ABS), acrylate and acrylic acid polymers such as polymethyl methacrylate, and other substituted and unsubstituted polyolefins, and copolymers thereof. Generally, at least one of the substrate or a portion of the SlipChip device comprises a biofouling-resistant polymer when the microdevice is employed to transport biological fluids. Polyimide is of particular interest and has proven to be a highly desirable substrate material in a number of contexts. Polyimides are commercially available, e.g., under the tradename Kapton®, (DuPont, Wilmington, Del.) and Upilex® (Ube Industries, Ltd., Japan). Polyetheretherketones (PEEK) also exhibit desirable biofouling resistant properties. Polymeric materials suitable for use with the invention include silicone polymers, such as polydimethylsiloxane, and epoxy polymers.

The SlipChip devices of the present invention may also be fabricated from a. “composite,” i.e., a composition comprised of unlike materials. The composite may be a block composite, e.g., an A-B-A block composite, an A-B-C block composite, or the like. Alternatively, the composite may be a heterogeneous combination of materials, i.e., in which the materials are distinct from separate phases, or a homogeneous combination of unlike materials. As used herein, the term “composite” is used to include a “laminate” composite. A “laminate” refers to a composite material formed from several different bonded layers of identical or different materials. Other preferred composite substrates include polymer laminates, polymer-metal laminates, e.g., polymer coated with copper, a ceramic-in-metal or a polymer-in-metal composite. One preferred composite material is a polyimide laminate formed from a first layer of polyimide such as Kapton®, that has been co-extruded with a second, thin layer of a thermal adhesive form of polyimide known as KJ®, also available from DuPont (Wilmington, Del.).

The device can be fabricated using techniques such as compression molding, injection molding or vacuum molding, alone or in combination. Sufficiently hydrophobic material can be directly utilized after molding. Hydrophilic material can also be utilized, but may require additional surface modification. Further, the device can also be directly milled using CNC machining from a variety of materials, including, but not limited to, plastics, metals, and glass. Microfabrication techniques can be employed to produce the device with sub-micrometer feature sizes. These include, but are not limited to, deep reactive ion etching of silicon, KOH etching of silicon, and HF etching of glass. Polydimethylsiloxane devices can also be fabricated using a machined, negative image stamp. In addition to rigid substrates, flexible, stretchable, compressible and other types of substrates that may change shape or dimensions may be used as materials for certain embodiments of the Slip Chip. In certain embodiments, these properties may be used to, for example, control or induce slipping.

In some instances, the base, plate and substrate of the SlipChip device may be made from the same material. Alternatively, different materials may be employed. For example, in some embodiments the base and plate may be comprised of a ceramic material and the substrate may be comprised of a polymeric material.

In some embodiments, the SlipCip device can be modified to include four etched circles that direct the placement of the four red alignment markers. In some embodiments, the device can contain from about 10 to about 10,000 small containers to hold the sample. Prior to attaching the two sides of the device, the containers can be located on either side of the chip. In some embodiments, about 1,000 to about 2,000 containers are used on either half of the chip. In some embodiments, each container has a volume of 4 to 10 nL. In some embodiments, when the two halves are manipulated to combine the reagents and initiate reactions, 10 to 10,000 individual reactions are initiated. In some embodiments, 600 to 2,000 individual reactions are initiated.

In some embodiments, other features may be included on the device to ensure proper manipulation including, but not limited to, for example: detection of proper and complete filling, detection of proper slipping between the plate and the base, detection of errors during slipping, detection of an expired or defective device, detection of bad reagents, etc. for example.

The SlipChip device may contain electrically conductive material. The material may be formed into at least one area or patch of any shape to form an electrode. The at least one electrode may be positioned on one surface on the base such that in a first position, the at least one electrode is not exposed to at least one first area on the opposing surface on the plate, but when the two parts of the device, base and plate, are moved relative to one another to a second position, the at least one electrode overlaps the at least one area. The at least one electrode may be electrically connected to an external circuit. The at least one electrode may be used to carry out electrochemical reactions for detection and/or synthesis. If a voltage is applied to at least two electrodes that are exposed to a substance in an area or a plurality of areas in fluidic communication or a combination of areas and ducts in fluidic communication, the resulting system may be used to carry out electrophoretic separations, and/or electrochemical reactions and/or transport. Optionally, at least one duct and/or at least one area may be present on the same surface as the at least one electrode and may be positioned so that in a first position, none of the at least one duct and the at least one electrode are exposed to an area on the opposing surface, but when the two parts of the device, base and plate, are moved relative to one another to a second position, the at least one duct and/or at least one area and the at least one electrode overlaps the at least one area.

In some embodiments the elements of an sample containing device, e.g. the SlipChip, are configured to be imageable by a camera, e.g. a iPhone. For example, high contrast materials can be used. For example, components can be constructed to be visible in a single plane. In some embodiments of the windows or transparent materials are used to allow imaging from a predetermined orientation. By imaging various components of the device a image can be generated which can be used to determine if the device is in suitable condition for further analysis. In some embodiments a computer is configured to determine whether components of the device are in proper orientation for analysis of an image to analyze a sample.

Several embodiments of the current invention require movement of a substance through, into, and/or across at least one duct and/or area. For example movement of a substance can be used for washing steps in immunoassays, removal of products or byproducts, introduction of reagents, or dilutions.

Loading of a substance may be performed by a number of methods, as described herein. Loading may be performed either to fill the ducts and areas of the device, for example by designing the outlets to increase flow resistance when the substance reaches the outlets. This approach is valuable for volume-limited samples or to flow the excess volume through the outlets, while optionally capturing analyte from the substance. Analytes can be essentially any discrete material which can be flowed through a microscale system Analyte capture may be accomplished for example by preloading the areas of the device with capture elements that are trapped in the areas (such as particles, beads or gels, retained within areas via magnetic forces or by geometry or with relative sizes of beads and ducts or with a membrane), thus whatever absorbs, adsorbs, or reacts with these beads or gels is also trapped. These areas will then retain an amount or component or analyte of the substances they are exposed to. This can also be done by functionalization of the surface of an area, deposition of a material on an area, attaching a monomer in a polymerization reaction (such as peptide or DNA synthesis) to an area, etc.

Other examples of capture elements include antibodies, affinity-proteins, aptamers, beads, particles and biological cells. Beads may be for example, polymer beads, silica beads, ceramic beads, clay beads, glass beads, magnetic beads, metallic beads, inorganic beads, and organic beads can be used. The beads or particles can have essentially any shape, e.g., spherical, helical, irregular, spheroid, rod-shaped, cone-shaped, disk shaped, cubic, polyhedral or a combination thereof. Capture elements are optionally coupled to reagents, affinity matrix materials, or the like, e.g., nucleic acid synthesis reagents, peptide synthesis reagents, polymer synthesis reagents, nucleic acids, nucleotides, nucleobases, nucleosides, peptides, amino acids, monomers, cells, biological samples, synthetic molecules, or combinations thereof. Capture elements optionally serve many purposes within the device, including acting as blank particles, dummy particles, calibration particles, sample particles, reagent particles, test particles, and molecular capture particles, e.g., to capture a sample at low concentration. Additionally the capture elements may be used to provide particle retention elements. Capture elements are sized to pass or not pass through selected ducts or membranes (or other microscale elements). Accordingly, particles or beads will range in size depending on the application.

A substance may be introduced to fill the majority of reaction areas and ducts. Filling may be continued further to provide excess sample, larger than the volume of areas and ducts. Introducing a volume of substance which is greater than the volume of areas and ducts will increase the amount of analyte which may be captured within the capture. Introducing a wash fluid after the introduction of a substance may be performed to wash the capture elements and analytes which are bound to the capture elements. Subsequent further slipping may be performed to conduct reactions and analysis of the analytes.

The approach described above is beneficial when analyzing samples with low concentrations of analytes, for example rare nucleic acids or proteins, markers and biomarkers of genetic or infectious disease, environmental pollutants, etc. (See e.g., U.S. Ser. No. 10/823,503, incorporated herein by reference). Another example includes the analysis of rare cells, such as circulating cancer cells or fetal cells in maternal blood for prenatal diagnostics. This approach may be beneficial fir rapid early diagnostics of infections by capturing and further analyzing microbial cells in blood, sputum, bone marrow aspirates and other bodily fluids such as urine and cerebral spinal fluid. Analysis of both beads and cells may benefit from stochastic confinement (See e.g., PCT/US08/71374, incorporated herein by reference).

A barcode is an optical machine-readable representation of data or information. A barcode can be a linear barcode. Some non-limiting examples of linear barcodes include, Codabar, Code 25, Code 11, Code 39, Code 93, Code 128, Code 128A, Code 128B, Code 128C, CPC Binary, DUN 14, EAN 2, EAN 5, EAN-8, EAN-13, Facing Identification Mark, GS1-128, EAN 128, USC 128, GS1 DataBar, RSS, HIBC, HIBCC, Intelligent Mail barcode, ITF-14, JAN, Latent image barcode, MSI, Pharmacode, PLANET, Plessey, PostBar, POSTNET, RM4SCC/KIX, Telepen, U.P.C. for instance.

A barcode can be a two dimensional barcode, or matrix such as a QR code. Some non-limiting examples of linear barcodes include, 3-DI, ArrayTag, AugTag, Aztec Code, Small Aztec Code, Codablock, Code 1 Code 16k, Code 49, ColorCode, Color Construct Code, Compact Matrix Code, CP Code, CyderCode, d-touch, DataGlyphs, Data Matrix, Datastrip Code, digital paper, Dot Code A, EZcode, Grid Matrix Code, HD Barcode, High Capacity Color Barcode, HueCode, INTACTA.CODE, InterCode, JAGTAG, MaxiCode, mCode, MiniCode, MicroPDF417, MMCC, Nintendo e-reader#Dot code, Optar, PaperDisk, PDF417, PDMark, QR Code, QuickMark Code, Secure Seal, SmartCode, Snowflake Code, ShotCode, SPARQCode, Stickybits, SuperCode, Trillcode, UltraCode, UnisCode, VeriCode, VSCode, WaterCode. A barcode can be a three dimensional such as a holograph.

One or more barcodes can be attached to the sample. One or more barcodes can be attached to a device that contains a portion of the sample. A barcode can be attached to a container that holds at least a portion of the sample. The barcode can be embedded within the material of an object or device that can hold the sample. In some embodiments, a barcode can be on the surface of an object or device that holds the sample. The barcode can be permanently affixed, reversibly attached, engraved, etched, drawn, or printed.

In some embodiments, a device can comprise a plurality of spatially-distinct analysis regions, wherein each analysis region holds a portion of the sample. In these embodiments, the machine-readable representation of data can be the shape, color, quantity, and/or spatial distribution the analysis regions on the device, for instance.

A barcode can contain data or information regarding the sample. The information regarding the sample can include information such as the date, time, and/or location from which the sample was obtained. A barcode can contain information regarding the organism from which the sample was obtained. In some embodiments, the sample can be obtained from a person, and a barcode can contain information regarding the person's name, the person's age, the person's weight, the person's height, time of sample collection, type of cells in sample, type of bodily fluid in sample, concentration of sample, batch number of sample, name of medical provider, expected results, previous sample information and/or other medical records.

A barcode can contain information regarding the contents of device to which it attached, for instance: the number, color, and/or spatial distribution of analysis regions on or within the device. A barcode can contain information regarding the contents of the analysis regions, for instance: the types of reagents or chemical species, enzymes, dyes, solvents, and/or nucleic acids. The barcode can contain information regarding the amplification of nucleic acids in the sample for instance: reaction time, reaction temperature, identification of reagents present, quantity of reagents.

It is to be understood that the exemplary methods and systems described herein may be implemented in various forms of hardware, software, firmware, special purpose processors, or a combination thereof. These instructions and programs can be executed by and/or stored on non-transitory computer readable media. Methods herein can be implemented in software as an application program tangibly embodied on one or more program storage devices. The application program may be executed by any machine, device, or platform comprising suitable architecture. It is to be further understood that, because some of the systems and methods depicted in the Figures are implemented in software, the actual connections between the system components (or the process steps) may differ depending upon the manner in which the present invention is programmed.

Background correction can be performed using software. In some embodiments a first image or series of images is taken to establish the amount of background, e.g. an amount of ambient light or auto fluorescence. This image or images can be used to correct for background in in an image of a sample. In some embodiments the first image or images are taken prior to taking the image or images of a sample. In some embodiments the first image or images are taken contemporaneously to taking the image or images of a sample. In some examples the first image or images are taken by using a separate set of detectors (e.g. detectors in a different wavelength) or using a separate set of filters. For example a green channel can be used to detect and correct for background when a red channel is being used to image a sample.

An image and/or a processed image and/or resulting data can be transmitted to a centralized computer for further analysis, e.g. for background correction.

Shape detection can be performed using one or more shapes to determine image fidelity. For example the shape of a well can be imaged and compared to a predicted shape. This comparison can be used to determine the quality of the imaging. Shape detection using one or more shapes can be used to determine the region to be analyzed. For example the boundary of a well can be determined prior to analysis. Shape detection using one or more algorithms to determine positive regions on an imaging device.

Processing and/or analyzing images and/or data analysis can take place on a centralized computer. Processing and/or analyzing images and/or data analysis can take place on a cloud computer Processing and/or analyzing images and/or data analysis can take place on the same device that performs the imaging, e.g. a cell phone.

In some embodiments the images and/or data are archived locally or on a remote database. The archived images can be used, for example, to check for quality of a batch or lot of devices which have been distributed to multiple users. In some embodiments quality control data is assessed free of information related to the source of a sample, e.g. any personally identifying data can be removed prior to analysis of the data for quality control.

Applying Poisson statistical analysis to quantify the number of fluorescent and non-fluorescent regions. Combining the results from wells of different volumes fully minimizes the standard error and provides high-quality analysis across a very large dynamic range. Recognizing two different concentrations and take into account both false positives and false negatives.

Applying Poisson statistical analysis to quantify concentration based on the number of fluorescent and non-fluorescent regions

The computer components, software modules, functions, data stores and data structures described herein may be connected directly or indirectly to each other in order to allow the flow of data needed for their operations. It is also noted that the meaning of the term module includes but is not limited to a unit of code that performs a software operation, and can be implemented for example as a subroutine unit of code, or as a software function unit of code, or as an object (as in an object-oriented paradigm), or as an applet, or in a computer script language, or as another type of computer code. The software components and/or functionality may be located on a single computer or distributed across multiple computers depending upon the situation at hand. In yet another aspect, a computer readable medium is provided including computer readable instructions, wherein the computer readable instructions instruct a processor to execute the methods described herein. The instructions can operate in a software runtime environment. In yet another aspect, a data signal is provided that can be transmitted using a network, wherein the data signal includes data calculated in a step of the methods described herein. The data signal can further include packetized data that is transmitted through wired or wireless networks. In an aspect, a computer readable medium comprises computer readable instructions, wherein the instructions when executed carry out a calculation of the probability of a medical condition in a patient based upon data obtained from the sample. The computer readable instructions can operate in a software runtime environment of the processor. In some embodiments, a software runtime environment provides commonly used functions and facilities required by the software package. Examples of a software runtime environment include, but are not limited to, computer operating systems, virtual machines or distributed operating systems although several other examples of runtime environment exist. The computer readable instructions can be packaged and marketed as a software product, app, or part of a software package. For example, the instructions can be packaged with an assay kit.

The computer readable medium may be a storage unit. Computer readable medium can also be any available media that can be accessed by a server, a processor, or a computer. The computer readable medium can be incorporated as part of the computer-based system, and can be employed for a computer-based assessment of a medical condition.

In some embodiment, the calculations described herein can be carried out on a computer system. The computer system can comprise any or all of the following: a processor, a storage unit, software, firmware, a network communication device, a display, a data input, and a data output. A computer system can be a server. A server can be a central server that communicates over a network to a plurality of input devices and/or a plurality of output devices. A server can comprise at least one storage unit, such as a hard drive or any other device for storing information to be accessed by a processor or external device, wherein the storage unit can comprise one or more databases. In an embodiment, a database can store hundreds to millions of data points corresponding to a data from hundreds to millions of samples. A storage unit can also store historical data read from an external database or as input by a user. In an embodiment, a storage unit stores data received from an input device that is communicating or has communicated with the server. A storage unit can comprise a plurality of databases. In an embodiment, each of a plurality of databases corresponds to each of a plurality of samples. In another embodiment, each of a plurality of databases corresponds to each of a plurality of different imaging devices, for example different consumer based cell phones. An individual database can also comprise information for a plurality of possible sample containment units. Further, a computer system can comprise multiple servers. A processor can access data from a storage unit or from an input device to perform a calculation of an output from the data. A processor can execute software or computer readable instructions as provided by a user, or provided by the computer system or server. The processor may have a means for receiving patient data directly from an input device, a means of storing the subject data in a storage unit, and a means for processing data. The processor may also include a means for receiving instructions from a user or a user interface. The processor may have memory, such as random access memory. In one embodiment, an output that is in communication with the processor is provided. After performing a calculation, a processor can provide the output, such as from a calculation, back to, for example, the input device or storage unit, to another storage unit of the same or different computer system, or to an output device. Output from the processor can be displayed by data display. A data display can be a display screen (for example, a monitor or a screen on a digital device), a print-out, a data signal (for example, a packet), an alarm (for example, a flashing light or a sound), a graphical user interface (for example, a webpage), or a combination of any of the above. In an embodiment, an output is transmitted over a network (for example, a wireless network) to an output device. The output device can be used by a user to receive the output from the data-processing computer system. After an output has been received by a user, the user can determine a course of action, or can carry out a course of action, such as a medical treatment when the user is medical personnel. In some embodiments, an output device is the same device as the input device. Example output devices include, but are not limited to, a telephone, a wireless telephone, a mobile phone, a PDA, a flash memory drive, a light source, a sound generator, a computer, a computer monitor, a printer, and a webpage. The user station may be in communication with a printer or a display monitor to output the information processed by the server.

A client-server, relational database architecture can be used in embodiments of the invention. A client server architecture is a network architecture in which each computer or process on the network is either a client or a server. Server computers are typically powerful computers dedicated to managing disk drives (file servers), printers (print servers), or network traffic (network servers). Client computers include PCs (personal computers), cell phones, or workstations on which users run applications, as well as example output devices as disclosed herein. Client computers rely on server computers for resources, such as files, devices, and even processing power. In some embodiments of the invention, the server computer handles all of the database functionality. The client computer can have software that handles all the front-end data management and can also receive data input from users.

Subject data can be stored with a unique identifier for recognition by a processor or a user. In another step, the processor or user can conduct a search of stored data by selecting at least one criterion for particular patient data. The particular patient data can then be retrieved. Processors in the computer systems can perform calculations comparing the input data to historical data from databases available to the computer systems. The computer systems can then store the output from the calculations in a database and/or communicate the output over a network to an output device, such as a webpage, a text, or an email. After a user has received an output from the computer system, the user can take a course of medical action according to the output. For example, if the user is a physician and the output is a probability of cancer above a threshold value, the physician can then perform or order a biopsy of the suspected tissue. A set of users can use a web browser to enter data from a biomarker assay into a graphical user interface of a webpage. The webpage is a graphical user interface associated with a front end server, wherein the front end server can communicate with the user's input device (for example, a computer) and a back end server. The front end server can either comprise or be in communication with a storage device that has a front-end database capable of storing any type of data, for example user account information, user input, and reports to be output to a user. Data from each user can be then be sent to a back end server capable of manipulating the data to generate a result. For example, the back end server can calculate a corrections for similar cell phones or compile data generated from similar sample collection units. The back end server can then send the result of the manipulation or calculation back to the front end server where it can be stored in a database or can be used to generate a report. The results can be transmitted from the front end server to an output device (for example, a computer with a web browser or a cell phone) to be delivered to a user. A different user can input the data and receive the data. In an embodiment, results are delivered in a report. In another embodiment, results are delivered directly to an output device that can alert a user.

The information from the assay can be quantitative and sent to a computer system of the invention. The information can also be qualitative, such as observing patterns or fluorescence, which can be translated into a quantitative measure by a user or automatically by a reader or computer system. In an embodiment, the subject can also provide information other than sample assay information to a computer system, such as race, height, weight, age, gender, eye color, hair color, family medical history, identity, location and any other information that may be useful to the user.

In some embodiments additional information is provided by sensors associated with the device. For example global positioning data, acceleration data, air pressure, or moisture levels may be measured by a device comprising the image sensor. This additional information can be used by the computer systems of the invention.

Information can be sent to a computer system automatically by a device that reads or provides the data from image sensor. In another embodiment, information is entered by a user (for example, the subject or medical professional) into a computer system using an input device. The input device can be a personal computer, a mobile phone or other wireless device, or can be the graphical user interface of a webpage. For example, a webpage programmed in JAVA can comprise different input boxes to which text can be added by a user, wherein the string input by the user is then sent to a computer system for processing. The subject may input data in a variety of ways, or using a variety of devices. Data may be automatically obtained and input into a computer from another computer or data entry system. Another method of inputting data to a database is using an input device such as a keyboard, touch screen, trackball, or a mouse for directly entering data into a database.

In an embodiment, a computer system comprises a storage unit, a processor, and a network communication unit. For example, the computer system can be a personal computer, laptop computer, or a plurality of computers. The computer system can also be a server or a plurality of servers. Computer readable instructions, such as software or firmware, can be stored on a storage unit of the computer system. A storage unit can also comprise at least one database for storing and organizing information received and generated by the computer system. In an embodiment, a database comprises historical data, wherein the historical data can be automatically populated from another database or entered by a user.

In an embodiment, a processor of the computer system accesses at least one of the databases or receives information directly from an input device as a source of information to be processed. The processor can perform a calculation on the information source, for example, performing dynamic screening or a probability calculation method. After the calculation the processor can transmit the results to a database or directly to an output device. A database for receiving results can be the same as the input database or the historical database. An output device can communicate over a network with a computer system of the invention. The output device can be any device capable delivering processed results to a user.

Communication between devices or computer systems of the invention can be any method of digital communication including, for example, over the internet. Network communication can be wireless, ethernet-based, fiber optic, or through fire-wire, USB, or any other connection capable of communication. In an embodiment, information transmitted by a system or method of the invention can be encrypted.

It is further noted that the systems and methods may include data signals conveyed via networks (for example, local area network, wide area network, internet), fiber optic medium, carrier waves, wireless networks for communication with one or more data processing or storage devices. The data signals can carry any or all of the data disclosed herein that is provided to or from a device.

Additionally, the methods and systems described herein may be implemented on many different types of processing devices by program code comprising program instructions that are executable by the device processing subsystem. The software program instructions may include source code, object code, machine code, or any other stored data that is operable to cause a processing system to perform methods described herein. Other implementations may also be used, however, such as firmware or even appropriately designed hardware configured to carry out the methods and systems described herein.

A computer system may be physically separate from the instrument used to obtain values from the subject. In an embodiment, a graphical user interface also may be remote from the computer system, for example, part of a wireless device in communication with the network. In another embodiment, the computer and the instrument are the same device.

An output device or input device of a computer system can include one or more user devices comprising a graphical user interface comprising interface elements such as buttons, pull down menus, scroll bars, fields for entering text, and the like as are routinely found in graphical user interfaces known in the art. Requests entered on a user interface are transmitted to an application program in the system (such as a Web application). In one embodiment, a user of user device in the system is able to directly access data using an HTML interface provided by Web browsers and Web server of the system.

A graphical user interface may be generated by a graphical user interface code as part of die operating system or server and can be used to input data and/or to display input data. The result of processed data can be displayed in the interface or a different interface, printed on a printer in communication with the system, saved in a memory device, and/or transmitted over a network. A user interface can refer to graphical, textual, or auditory information presented to a user and may also refer to the control sequences used for controlling a program or device, such as keystrokes, movements, or selections. In another example, a user interface may be a touch screen, monitor, keyboard, mouse, or any other item that allows a user to interact with a system of the invention.

In yet another aspect, a method of taking a course of medical action by a user is provided including initiating a course of medical action based on sample analysis. The course of medical action can be delivering medical treatment to said subject. The medical treatment can be selected from a group consisting of the following: a pharmaceutical, surgery, organ resection, and radiation therapy. The pharmaceutical can include, for example, a chemotherapeutic compound for cancer therapy. The course of medical action can include, for example, administration of medical tests, medical imaging of said subject, setting a specific time for delivering medical treatment, a biopsy, and a consultation with a medical professional. The course of medical action can include, for example, repeating a method described above. A method can further include diagnosing the medical condition of the subject by said user with said sample. A system or method can involve delivering a medical treatment or initiating a course of medical action. If a disease has been assessed or diagnosed by a method or system of the invention, a medical professional can evaluate the assessment or diagnosis and deliver a medical treatment according to his evaluation. Medical treatments can be any method or product meant to treat a disease or symptoms of the disease. In an embodiment, a system or method initiates a course of medical action. A course of medical action is often determined by a medical professional evaluating the results from a processor of a computer system of the invention. For example, a medical professional may receive output information that informs him that a subject has a 97% probability of having a particular medical condition. Based on this probability, the medical professional can choose the most appropriate course of medical action, such as biopsy, surgery, medical treatment, or no action. In an embodiment, a computer system of the invention can store a plurality of examples of courses of medical action in a database, wherein processed results can trigger the delivery of one or a plurality of the example courses of action to be output to a user. In an embodiment, a computer system outputs information and an example course of medical action. In another embodiment, the computer system can initiate an appropriate course of medical action. For example, based on the processed results, the computer system can communicate to a device that can deliver a pharmaceutical to a subject. In another example, the computer system can contact emergency personnel or a medical professional based on the results of the processing. Courses of medical action a patient can take include self-administering a drug, applying an ointment, altering work schedule, altering sleep schedule, resting, altering diet, removing a dressing, or scheduling an appointment and/or visiting a medical professional. A medical professional can be for example a physician, emergency medical personnel, a pharmacist, psychiatrist, psychologist, chiropractor, acupuncturist, dermatologist, urologist, proctologist, podiatrist, oncologist, gynecologist, neurologist, pathologist, pediatrician, radiologist, a dentist, endocrinologist, gastroenterologist, hematologist, nephrologist, ophthalmologist, physical therapist, nutritionist, physical therapist, or a surgeon.

The image can be uploaded to the cloud. In some embodiments, the image can be automatically uploaded to the cloud without user interaction. The images uploaded to the cloud can be sent to one or more local computers or devices. The images can be synced between multiple computers and/or devices. The uploading and syncing of images can be controlled by softward. For instance, the Symbian software on which the Nokia 808 camera runs has access to the cloud-based storage service Skydrive, produced by Microsoft, and the uploaded files are then instantly synced with all computers that have the Skydrive application installed and are logged into the same account. The can be accomplished on other platforms. For instance, the images can be automatically uploaded to the cloud and synced using Android or iOS architectures. Non-limiting examples of existing software solutions include box.net, dropbox, skydrive, and iCloud. By using a cloud-based architecture for the automatic transfer of images from the mobile device to a computer, virtually any available smartphone on the market can be tied into our automatic analysis software without any fine-tuning or tweaking of the software for the various operating systems and handsets available on the market today. Using a cloud-based service to extract the images from the cell phone can allow for easy archiving and traceability of the images and raw data.

In some embodiments, the images are maintained on the device comprising the image sensor, and not sent to the cloud or synced. Software can be written to do direct image analysis on the device comprising the image sensor. Handling the processed images offsite also allows for the saving of the processed images without having to deal with bandwidth for transmitting those from the phone, or having a cell phone with a limited size run out of room for additional files. Partial or complete image processing on the cell phone can also be directly performed.

Image analysis is performed in a custom written Labview program with the following workflow. Once an image is taken on the cell phone, it is automatically transferred to any computer in the world via the Skydrive cloud. Meanwhile, the Labview program has been written to “watch” any folder on the computer for new files that fit into a specific filtered category (i.e., *.jpg, *.png, *.tiff) and automatically analyze those files. The program is multithreaded such that the “watcher” and the “analyzer” of the software can run simultaneously without disruption. Upon a new file being added to the watched folder (via cloud syncing), it is added to a queue that the analyzer watches. The queue can have multiple files waiting in it, so it is not a problem if images are being photographed faster than the software can handle, or in the case of simply adding to the watched folder a set of files that have not previously been analyzed. Thus the analysis software is not tied to any specific platform either and can be easily modified to analyze images from any device whether it be cellular phone, compact camera, dslr, microscope, etc.

Once the uploaded file has been added to the queue, it enters the analysis portion of the software. The software will then take the RGB image and split it into three channels based on color. In our case, the blue channel is not used, as that color is filtered out before reaching the CMOS imaging sensor. The devices have been etched with four 4 mm-diameter circles, each of which has a piece of red tape that has been cut to those dimensions placed on them. The tape is red so that it does not interfere with the fluorescence imaging, which is green. These 4 circles are then used to determine if the full image has been taken by searching for 4 different circles of a certain size in the red channel. The circles are then sorted in a way that the software can understand, before then having any tilt in the image be corrected by rotating the image until the line between two dots are parallel to the image axis. After this correction, the portion of the chip that contains the wells is then determined based upon distances from the dots.

As we are using calcein as the fluorescent compound, the fluorescence signal shows up in the green channel, and the red channel contains the scattered light pattern. Therefore, we can use a normalized subtraction of the red channel from the green channel to obtain a background corrected image of the positive wells. The image is then filtered in three different ways to increase the intensity of the positive wells before thresholding, namely, an averaging filter to blur out any overexposed pixels, a detail-highlighting filter to make the positive wells brighter, and then a median filter to drop the intensity of the negative wells. A threshold is then performed to remove the majority of the negative wells from the image, followed by an algorithm to remove small defects. The image is then converted back from binary using a lookup table before doing a pattern match against the features left in the portion of the image that has been determined previously to contain wells to determine which are positive.

Once the number of positive wells has been determined, that number is processed using Poisson statistics and prior knowledge about the chip in question to determine the original concentration of sample in the chip. This information is then automatically sent via email to any valid email account and is then received by the original person who took the image regardless of where they are in the world relative to the computer that performs the image analysis. The time that elapses between the taking of the image and the receipt of email confirmation has been performed in well under 1 minute, although actual time is subject to the upload speed on the network of the cell phone and download speed on the network of the computer. This is important, because if an error is detected in the course of an analysis, such as not being able to find all 4 spots, the user needs to be quickly alerted that another image must be taken. The software has been programmed to do such, and the user typically knows in under 1 minute to take another image. Having the ability to notify by email can give the ability to notify via text. Cell phone providers can have a service that will send the body of an email as a text to specific users. Other servers that can be leveraged as SMS messengers. The analysis process can use computer automation to notify a user if the image can be used. The notification can be an SMS message, email message, phone call, web posting, or electronic message for example. In some embodiments, the amount of time from the uploading of the image until the user is notified can be referred to as the analysis process. The analysis process can take less than 5 min, 4 min, 3 min, 2 min, 1 min, 50 sec, 45 sec, 40 sec, 30 sec, 20 sec, 10 sec, 9 sec, 8 sec, 7 sec, 6 sec, 5 sec, 4 sec, 3 sec, 2 sec, 1 sec, 0.5 sec, 0.4 sec, 0.3 sec, 0.2 sec, or 0.1 sec, for example. In some embodiments, the analysis process takes less than 1 min.

At least one calibration source for providing a calibration emission, and at least one calibration photodiode for sensing the calibration emission wherein the control circuitry has a differential circuit for subtracting the calibration photodiode output from each of the detection photodiode outputs.

A communication interface can be a universal serial bus (USB) connection such that the outer casing is configured as a USB drive.

In some instances the information is transmitted back to the mobile device which was used for imaging. For example a image may be obtained, send to a separate computer for analysis, and then the image or date related to the image can be transmitted back to the mobile device. In some embodiments an image and/or a processed image and/or resulting data the user is transmitted to a separate device, e.g. a physicians mobile device may receive the information. In some instances two or sets of information are transmitted to two or more devices. The two or more sets of information can be the same information, or in some embodiments, separate data is sent to each user. For example a patient may receive some information related to an image while the patient's doctor receives information more suitable for a physician's analysis.

While offloading the analysis of images to “the cloud” provides a number of benefits, including traceability and archiving of raw data, global access, and compatibility with virtually all smartphone operating systems, it requires a wireless data connection of sufficiently high bandwidth; thus, direct on-phone analysis could be preferable in some scenarios.

In some embodiments chemical heaters are used to heat the sample. For example a chemical heater can heat a sample containing device (e.g. a SlipChip) prior to or during imaging. Chemical heaters can function using a exothermal reaction. Exothermic reaction are reactions that produce heat, e.g. Mg+2H2O→Mg(OH)2+H2+heat, CaO(s)+H2O(l)→Ca(OH)2(s), or CaO(s)+H2O(l)→Ca(OH)2(s). The reaction can comprise mixed metallic iron particles and table salt (NaCl) with the magnesium particles (see e.g. U.S. Pat. Nos. 4,017,414 and 4,264,362). In some embodiments a chemical heater is capable of being imaged and can have indicia of whether heating has appropriately occurred.

A kit can include a SlipChip device, and a supply of a reagent selected to participate in nucleic acid amplification. In some embodiments, the reagent can be disposed in a container adapted to engage with a conduit of the first component, the conduit of the second component, Of both. Such a container can be a pipette, a syringe, and the like. In some embodiments, the kit includes a heater.

In some embodiments, the devices and/or kits can also include a device capable of supplying or removing heat from the first and second components. Such devices include heaters, refrigeration devices, infrared or visible light lamps, and the like, some embodiments, the kit can also include a device capable of collecting an image of at least some of the first population of wells, the second population of wells, or both. In some embodiments, the device includes a mobile communication device or a tablet. In some embodiments, the kit can include accessories that would aid the device in collecting an image. In some embodiments, the kit can include codes that allow access to software for analysis over a mobile device or tablet. In some embodiments, a kit comprises a SlipChip, reagents for an amplification reaction, and instructions to process a sample. In some embodiments, a kit comprises a SlipChip, reagents for an amplification reaction, software to carry out the imaging of a sample, and instructions to process a sample.

Some embodiments of the device use a homogeneous protein detection assay to detect specific proteins within a crude cell lysate or purified protein in certain buffer. These assays can utilize antibodies or aptamers to capture the target protein.

In one type of assay, an aptamer which binds to a particular protein is labeled with two different fluorophores or luminophores and which can function as a donor and an acceptor in a fluorescence resonance energy transfer (FRET) or electrochemiluminescence resonance energy transfer (ERET) reaction. Both donor and acceptor are linked to the same aptamer, and the change in separation is caused by a change in conformation upon binding to the target protein. For example, an aptamer in the absence of the target forms a conformation where the donor and acceptor are in close proximity; upon binding to the target, the new conformation results in a larger separation between the donor and acceptor. When the acceptor is a quencher and the donor is a luminophore, the effect of binding to the target is an increase in light emission 250 or 862.

A second type of assay uses two antibodies or two aptamers that must independently bind to different, non-overlapping epitopes or regions of the target protein. These antibodies or aptamers are labelled with different fluorophores or luminophores and which can function as a donor and an acceptor in a fluorescence resonance energy transfer (FRET) or electrochemiluminescence resonance energy transfer (ERET) reaction. The fluorophores or luminophores and form part of a pair of short complementary oligonucleotides attached to the antibodies or aptamers via long, flexible linkers. Once the antibodies or aptamers bind to the target protein, the complementary oligonucleotides find each other and hybridize to one another. This brings the donors and acceptors and in close proximity to one another resulting in efficient FRET or ERET that is used as a signal for target protein detection.

To ensure there is no, or very little, background signal as a result of the oligonucleotides attached to the two antibodies or aptamers hybridizing to one another in the absence of their binding to the protein, it is necessary to carefully choose the length and sequence of the complementary oligonucleotides so that the dissociation constant (kd) for the duplex is relatively high (^{˜}5 μM). Thus when free antibodies or aptamers labelled with these oligonucleotides are mixed at nanomolar concentrations, well below that of their kd, the likelihood of duplex formation and a FRET or ERET signal being generated is negligible. However, when both antibodies or both aptamers bind to the target protein, the local concentration of the oligonucleotides will be much higher than their kd resulting in almost complete hybridization and generation of a detectable FRET or ERET signal.

Crude cell lysates are often turbid and may contain substances which autofluoresce. In such cases, the use of molecules with long-lasting fluorescence or electrochemiluminescence and donor-acceptor pairs and which are optimized to give maximal FRET or ERET is desired. One such pair is europium chelate and Cy5, which has previously been shown to significantly improve signal-to-background ratio in such a system when compared with other donor-acceptor pairs, by allowing the signal to be read after interfering background fluorescence, electrochemiluminescence or scattered light has decayed. Europium chelate and AlexaFluor or terbium chelate and Fluorescein FRET or ERET pairs also work well. The sensitivity and specificity of this approach is similar to that of enzyme-linked immunosorbent assays (ELISAs), but no sample manipulation is required.

Some embodiments of the device use a heterogeneous protein detection assay to detect specific proteins within a crude cell lysate or purified protein in certain buffer. These assays can utilize antibodies or aptamers to capture the target protein. One of the antibodies or one of the aptamers is attached to the base of the well or magnetic beads and the protein lysate is combined with the other antibody or aptamer during lysis within the chemical lysis section to facilitate binding to the first antibody or aptamer prior to entering the well. This increases the subsequent speed with which a detectable signal is generated as only one conjugation or hybridization event is required within the proteomic assay chamber. To generate a signal for readout, one or more enzyme molecules, fluorophores, oligos or nanoparticles are attached to the second antibody or aptamer. A signal is then generated which can, for example, be visualized as fluorescence, chemiluminescence, ability to scatter light, etc. (Rissin, David M., et al. “Simultaneous detection of single molecules and singulated ensembles of molecules enables immunoassays with broad dynamic range.” Analytical chemistry 816 (2011): 2279-2285.; Walt, David R. “Optical Methods for Single Molecule Detection and Analysis.” Analytical chemistry 85.3 (2012): 1258-1263.; Shon, Min Ju, and Adam E. Cohen. “Mass Action at the Single-Molecule Level.” Journal of the American Chemical Society 134.35 (2012): 14618-14623.; Kan, Cheuk W., et al. “Isolation and detection of single molecules on paramagnetic beads using sequential fluid flows in microfabricated polymer array assemblies.” Lab on a Chip 12.5 (2012): 977-985.; Zhang, Huaibin, et al. “Oil-sealed femtoliter fiber-optic arrays for single molecule analysis.” Lab on a chip 12.12 (2012): 2229-2239.)

Some embodiments of the device could be used to detect different biological targets such as, for example, proteins, bacteria, viruses, infectious agents etc., using nucleic acid labels. In some embodiments the target is tagged with an oligonucleotide which can be used for detection. The oligonucleotide tag can be further amplified using any one of a number of different nucleic acid amplification strategies, such as for example, PCR, LAMP, RPA, NASBA, RCA, etc. The oligonucleotide tag could also be visualized using fluorescent probes for example as shown by Chen (Huang, Suxian, and Yong Chen.

“Polymeric Sequence Probe for Single DNA Detection.” Analytical chemistry 83.19 (2011): 7250-7254.)

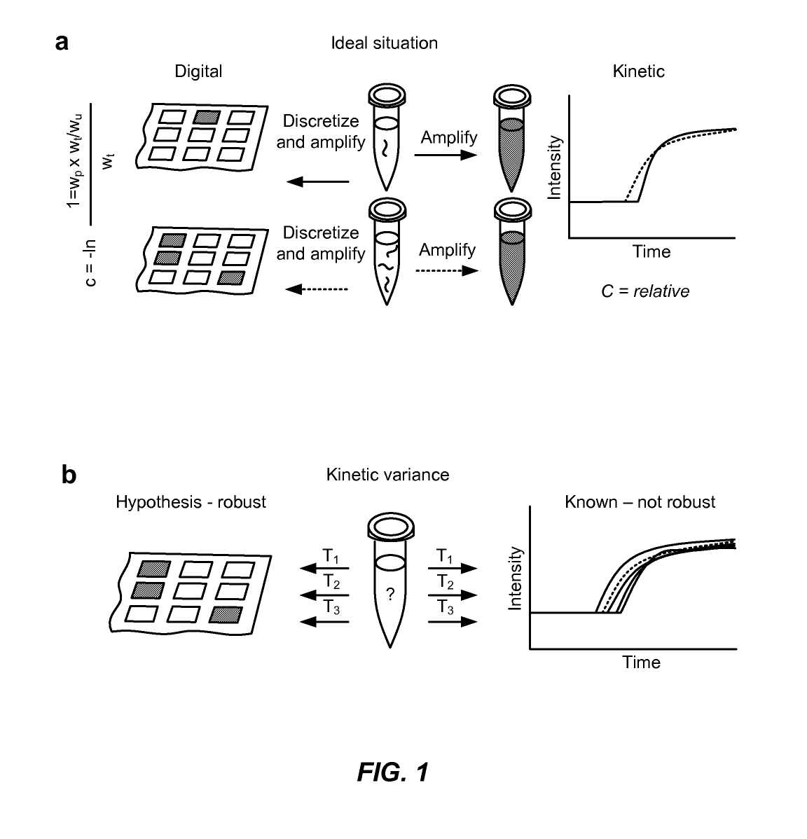

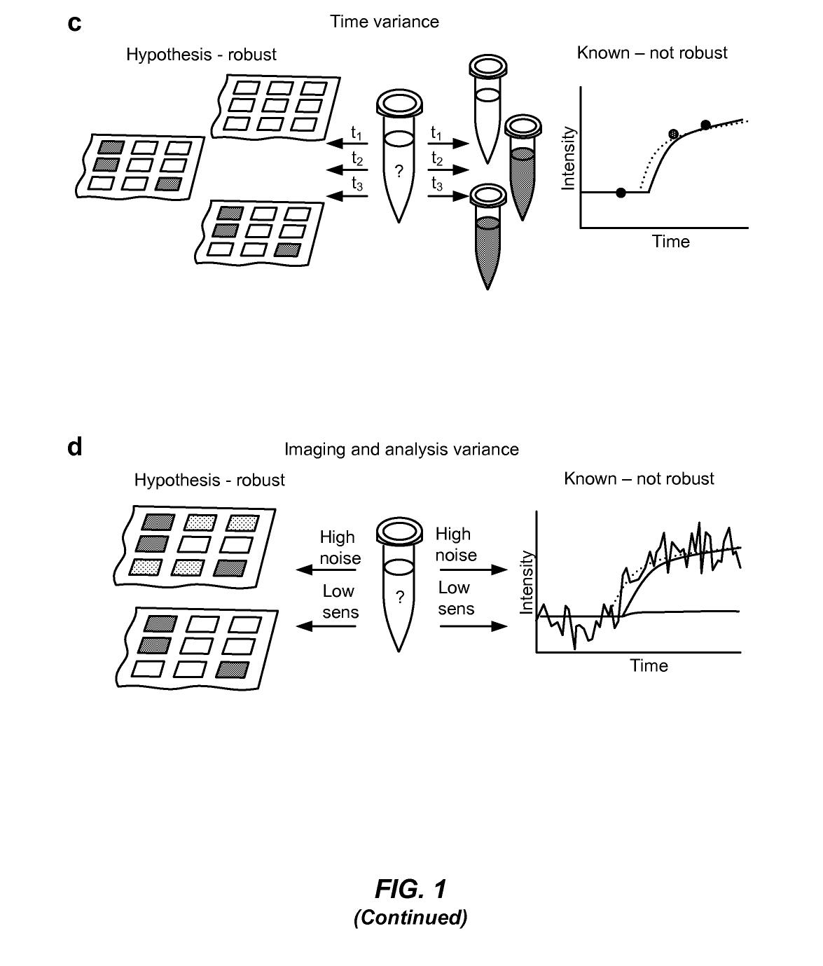

At present, the majority of quantitative analytical measurements are performed in a kinetic format, and known to be not robust to perturbation that affects the kinetics itself, or the measurement of kinetics. The inventors demonstrated that the same measurements performed in a “digital” (single-molecule) format show increased robustness to such perturbations (FIG. 1).

In some embodiments, the inventor selected HIV-1 RNA as a target molecule and selected isothermal digital reverse transcription-loop-mediated amplification (dRT-LAMP) as the amplification chemistry. LAMP amplification chemistry was chosen for three reasons: i) When performed with a qualitative readout, in at least one example it is known to tolerate a number of perturbations, so the question of robustness with a quantitative readout is a meaningful one; ii) While it is an autocatalytic, exponential amplification chemistry, its mechanism is sufficiently complex that it was not obvious whether its initiation phase or propagation phase, and therefore the digital or kinetic format, would be more affected by perturbations; iii) Digital LAMP has been recently demonstrated on various microfluidic platforms. The inventors used a microfluidic SlipChip device_ENREF_{—}41 because it is well-suited for simple confinement and amplification of single molecules, it is convenient for performing multi-step reactions on single molecules, and because it has been validated with dRT-LAMP. A two-step RT-LAMP protocol was used because it can be more efficient than one-step RT-LAMP for the specific sequences used. Also, RT-LAMP is an attractive amplification chemistry under limited resource settings because it does not require thermocycling equipment and can be run using chemical heaters that do not require electricity. Furthermore, it is compatible with highly fluorescent calcein-based readout chemistry.

In some embodiments, the present invention can be performed using any microfluidic platforms that support digital single-molecule manipulations. In some embodiments, the present invention can be applied to study of biological systems, e.g., robustness of circadian clocks to temperature fluctuations. In some embodiments, the present invention can be used for quantitative measurements under limited resource settings because it is ultra-rapid, specific, provide bright positive and dim negative signals, and is robust to experimental perturbations.

### EXAMPLES

These examples are provided for illustrative purposes only and not to limit the scope of the claims provided herein.

### Example 1

### Formation of a SlipChip

The procedure of fabricating desired glass SlipChips using soda lime glass was based on previous work. The two-step exposing-etching protocol was adapted to create wells of two different depths (5 μm for thermal expansion wells, 55 μm for all the other wells). After etching, the glass plates were thoroughly cleaned with piranha acid and DI water, and dried with nitrogen gas. The glass plates were then oxidized in a plasma cleaner for 10 minutes and immediately transferred into a desiccator for 1 hour of silanization. They were rinsed thoroughly with chloroform, acetone, and ethanol, and dried with nitrogen gas before use.

Plastic polycarbonate SlipChip devices were directly oxidized in a plasma cleaner for 15 minutes after they were received from microfluidic ChipShop GmbH, and then transferred into a desiccator for 90 minutes of silanization. They were soaked in tetradecane for 15 minutes at 65° C. and then rinsed thoroughly with ethanol, then dried with nitrogen gas before use. Plastic SlipChip devices were not reused.

The SlipChips were assembled under de-gassed oil (mineral oil: tetradecane 1:4 v/v; Fisher Scientific). Both top and bottom plates were immersed into the oil phase and placed face to face. The two plates were aligned under a stereoscope (Leica, Germany) as shown and fixed using binder clips. Two through-holes were drilled in the top plate to serve as fluid inlets. The reagent solution was loaded through the inlet by pipetting.

### Example 2

### Single-Molecule Amplification in a SlipChip

A digital reverse-transcription loop-mediated isothermal amplification (dRT-LAMP) reaction was used for quantifying HIV-1 viral load. LAMP produces a bright fluorescence signal through replacement of manganese with magnesium in calcein.

Digital LAMP experiments have been described previously. Primers targeting the p24 gene were used. Quantifying viral load is necessary to monitor the effectiveness of antiretroviral therapy (ART). HIV virus quickly mutates under pressure of drug therapy due to its error-prone reverse transcriptase, which converts viral RNA to cDNA. These multiple mutations allow for the sudden appearance of drug-resistant strains that could be controlled by switching to another ART.

The steps of a digital LAMP experiment include loading samples onto a SlipChip device consisted of two glass plates with etched wells and channels lubricated with a layer of hydrocarbon oil enabled loading, compartmentalize, incubate, and mixing of reagents. At first slip, a solution containing template, one of the primers, and RT enzyme was compartmentalized (stochastically confined) into wells after loading. This stochastic confinement effectively increases the concentration of active RNA concentration in each well, enabling the reaction to be very efficient in each well. cDNA was synthesized from RNA in each compartment during the reverse transcription step. After a short incubation, a second slip allowed a second solution consisting of LAMP reagents with the rest of the primers to be loaded. Finally, LAMP reaction was initiated by the third slip, and the entire device was incubated at 63° C. for 1 hour.

For the 40-well SlipChip design, the concentration of each primer in solutions used for loading was 0.15 μM. The primer solution was flowed in Teflon tubing with 200 μm ID (Weico Wire & Cable Inc., Edgewood, N.Y.) ended with a thinner PTFE tubing with 50 μm ID (Zeus Industrial Products Inc., Raritan, N.J.). Solution was driven by 50 μL Hamilton glass syringe filled with tetradecane. A volume of 0.1 μL of primer solution, controlled by a Harvard syringe pump, was deposited into each circular well. PCR mix containing template solution in concentration of 100 pg/μL was injected to the channels for the reaction.

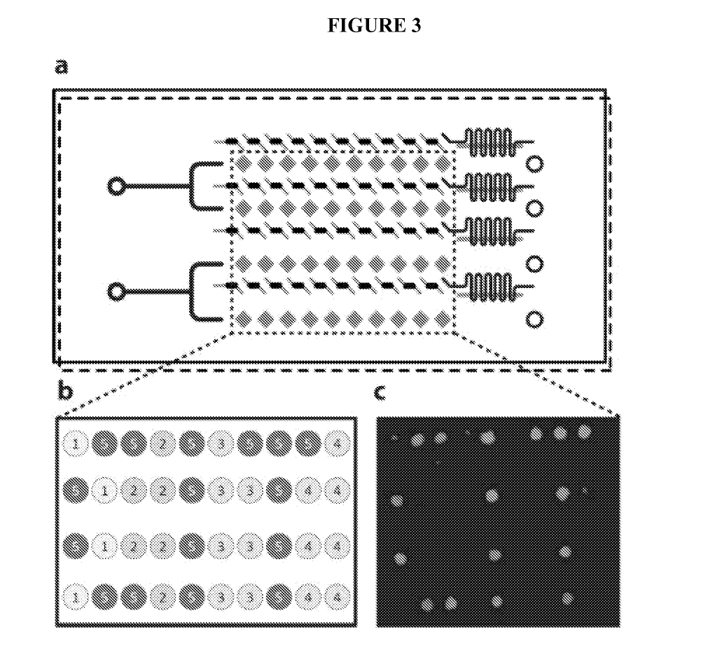

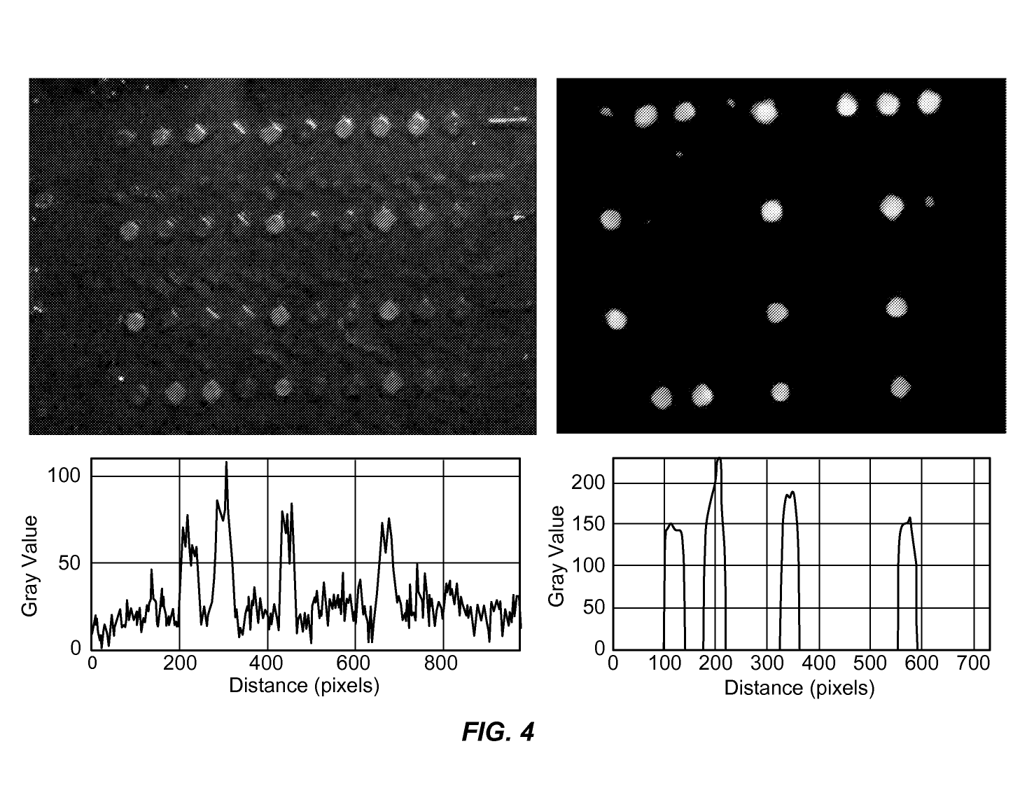

For the 40-well SlipChip design (FIG. 3), primer 1 was E. coli nlp gene (F: ATA ATC CTC GTC ATT TGC AG; R: GACTTC GGGTGA TTG ATA AG); primer 2 was Pseudomonas aeruginosa vic gene (F: TTC CCT CGC AGA GAA AAC ATC; R: CCT GGT TGA TCA GGT CGA TCT); primer 3 was Candida albicans calb (F: TTT ATC AAC TTG TCA CAC CAG A; R: ATC CCG CCT TAC CAC TAC CG); primer 4 was Pseu general 16S (F: GAC GGG TGA GTA ATG CCT A; R: CAC TGG TGT TCC TTC CTA TA); primer 5 was Staphylococcus aureus nuc gene (F: GCGATTGATGGTGATACGGTT; R:AGCCAAGCCTTGACGAACTAAAGC). Primers were ordered from Integrated DNA Technologies (Coralville, Iowa).

An initial step of 5 min at 95° C. was used to activate the enzyme for the reaction. Next, a total 38 cycles of amplification were performed as follows: a DNA denaturation step of 1 min at 95° C., a primer annealing step of 30 sec at 55° C., and a DNA extension step of 45 sec at 72° C. After the final cycle, the DNA extension step was performed for 5 min at 72° C. Then, the SlipChip was kept in the cycler at 4° C. before imaging.

### Example 3

### Imaging of SlipChip with Cell-Phone Camera

After incubation, the device from Example 2 was placed in a shoebox with a small window to mimic a dark room and imaged with a Nokia 808 cell phone.

A Nokia Pureview 808 cell phone was used to image and count microwells that contained the amplification product. This cell phone features a CMOS sensor with a Xenon flash which generates over 100,000 lux with a pulse with (PW) of 100-450 μs. The Nokia 808 PureView's large 1/1.4″ CMOS sensor has a 41 MP resolution, outputting a maximum of 38 MP (at 4:3 aspect ratio); pixel size is 1.4 μm. The camera has a Carl Zeiss F2.4 8.02 mm lens. Images captured in the PureView modes are created by oversampling from the sensor's full resolution. Pixel oversampling bins many pixels to create a much larger effective pixel, thus increasing the total sensitivity of the pixel.

The camera has focus distance of 15 cm in close-up mode, so a cell phone objective lens was used to bring the camera in close proximity to the imaged device. To excite fluorescence by the camera flash, two additive dichroic filters 1F1B (Thorlabs, Newton, N.J.) were placed in front of the cell phone's flash. These filters were >85% transmission for 390-480 nm and <1% for 540-750 nm, cut-off is 505±15 nm. To detect fluorescence, two green long-pass 5CGA-530 filters from Newport (Franklin, Mass.) were added to the objective lens. These filters had excellent blocking of >5OD and high transmission of >90% at wavelengths over 530 nm. Two excitation filters (FD1B) were stacked and attached in front of the camera flash. For fluorescence detection, two 5CGA-530 long-pass filters were inserted into magnetically mounted lens.

The highly reflective glass device was tilted by about 10 degrees relative to the cell phone lens-device axis to prevent direct reflection back to the objective and to force direct reflected light to go to the side due to tilt. Additionally, a black screen was added on the side of the device to block the scattered light from flash from oversaturating the CMOS sensor. Such geometry, combined with the color filters described above, allowed reaching S/N ratios close to 50.

### Example 4

### Image Sent to the Cloud and Synced to Other Devices

The image captured in Example 3 was initially stored on the cell phone. The Symbian software on which the Nokia 808 camera runs had access to the cloud-based storage service Skydrive, produced by Microsoft. This cloud-based service gave the option of automatically uploading, without user interaction, of all images taken with the phone to the service. This option was selected, and each image was automatically uploaded to the cloud-based storage service Skydrive, and instantly synced with all computers that have the Skydrive application installed and were logged into the same account.

### Example 5

### Processing Images on a Separate Device

A separate computer was configured to have a folder with proper login and password to receive files from the Skydrive account used in Example 4. With this configuration, each new image captured with the device of Example 3 was automatically transferred to this computer. The computer was additionally configured with a softward program written in Labview to detect new files in a folder, and to automatically analyze any new files that fit into a specific filtered category (i.e., *.jpg, *.png, *.tiff). The program was configured to detect and analyze images in the Skydrive Folder. The program was multithreaded such that the detection of the files and the analysis of the files can run simultaneously without disruption. Upon a new file being added to the watched folder (via cloud syncing), it was added to a queue that the analyzer watches. The queue was capable of having multiple files waiting in it, so it continues to function even when images are being photographed faster than the software can handle, or in the case of simply adding to the watched folder a set of files that have not previously been analyzed. Thus the analysis software was not tied to any specific platform either and can be easily modified to analyze images from any device whether it be cellular phone, compact camera, dslr, microscope, etc. The image file from Example 3 was synced to the computer running this software, and entered the queue. After the uploaded file was added to the queue, it entered the analysis portion of the software. The software took the image and split it into three monochrome 8-bit images for each individual color. The red-channel image was used to determine whether or not the entire chip had been imaged by searching for markers on the device (four red circles of tape, in this case). If all circles had been found, the image was then rotated such that the device was parallel to the top of the image box, removing any rotational bias. A background-corrected image was then generated by subtracting the red-channel monochrome image from the green-channel monochrome image, which contained the fluorescence information. The image was then subjected to a filtering process to increase the intensity of the positive wells. The filtering process included the following steps, in the following order: i) a 3×3 “local average” filter, ii) a 2×2 “median” filter, iii) an 11×11 “highlight details” filter, and iv) a 5×5 “median” filter. The filtered image was then thresholded using an entropy algorithm. After thresholding, a portion of the image (defined by the position of the markers) was analyzed and all individual spots were subjected to a size-filtering algorithm. This yielded the eventual total number of counts, which was then statistically transformed into a concentration before being emailed to the user or proper authority. The SlipChip device of Example 4 was etched with four 4 mm-diameter circles, each of which had a piece of red tape that has been cut to those dimensions placed on them. The tape was red so that it did not interfere with the fluorescence imaging, which is green. These 4 circles were then used to determine if the full image has been taken by searching for 4 different circles of a certain size in the red channel. The circles were then sorted in a way that the software can understand, before then having any tilt in the image be corrected by rotating the image until the line between two dots are parallel to the image axis. After this correction, the portion of the chip that contains the wells was then determined based upon distances from the dots.

The fluorescence signal from the calcein within the sample reaction emits in the green channel, and the red channel contains the scattered light pattern. Therefore, a normalized subtraction of the red channel from the green channel was used to obtain a background corrected image of the positive wells. The image was then filtered in three different ways to increase the intensity of the positive wells before thresholding, namely, an averaging filter to blur out any overexposed pixels, a detail-highlighting filter to make the positive wells brighter, and then a median filter to drop the intensity of the negative wells. A threshold was then performed to remove the majority of the negative wells from the image, followed by an algorithm to remove small defects.

### Example 6

### Determining the Conclusion Description from Processed Image

The image processed in Example 5, was then converted back from binary using a lookup table before doing a pattern match against the features left in the portion of the image that has been determined previously to contain wells to determine which are positive. Once the number of positive wells was determined, that number was processed using Poisson statistics and knowledge about the chip to determine the original concentration of sample in the chip.

### Example 7

### Error Alert Process

This information was then automatically sent via email to any valid email account and was then received by the original person who took the image regardless of where they were in the world relative to the computer that performed the image analysis. The time that elapsed between the taking of the image and the receipt of email confirmation had been performed in well under 1 minute, although actual time was subject to the upload speed on the network of the cell phone and download speed on the network of the computer. This was important, because if an error was detected in the course of an analysis, such as not being able to find all 4 spots, the user would need to be quickly alerted that another image must be taken. The software had been programmed to do such, and the user typically would know in under 1 minute to take another image. Text, SMS messengers and email were used as means of quickly alerting the user if an error was detected.

### Example 8

### General Workflow of Image Processing

A workflow for the processing of an image proceeds in the following steps. A raw image is acquired by cell phone. In step 1, based on the position of the four bright markers, the software recognizes the right region to be analyzed. In step 2, subtraction of the red from green channel occurs. In steps 3-5, a filtering algorithm takes place and an image is generated after processing. In step 6, “positives” counting take place. In step 7, a final image is generated with counted “positives”. If an error occurs, the user is altered via text, email or SMS messenger to retake the image.

### Example 9

### Fabrication of a SlipChip for dRT-LAMP and Multiplexed PC Experiments

The SlipChip was made from soda_lime glass plate coated with chromium and photoresist (relic Company, Valencia, Calif.). The glass plate was aligned with a photomask containing the design for the wells, and the AZ 1500 photoresist was exposed to UV light by following the standard protocol. Immediately after exposure, the areas of photoresist exposed to UV light were removed by 0.1 mol/L NaOH solution. A chromium etchant was applied to remove the exposed underlying chromium layer. Then, the glass plate was rinsed with Millipore water and dried with nitrogen gas. The glass plate was then immersed under a glass etching solution to etch the glass surface where chromium coating was removed in the previous steps. After etching, the glass plate was silanized with dichlorodimethylsilane (Sigma-Aldrich). The top and bottom plates of the SlipChip were assembled under degassed oil (mineral oil: tetradecane 1:4 v/v for dRT-LAMP and pure mineral oil for PCR). Both top and bottom plates were immersed into the oil phase and placed face to face. The two plates were aligned under a stereoscope (Leica, Germany) and fixed using binder clips. Through-holes were drilled into the top plate to serve as fluid inlets and oil outlets in dead-end filling. The reagent solutions were loaded through the inlets by pipetting.

A two-step exposing-etching protocol was adapted to create wells of two different depths (5 μm for thermal expansion wells and 55 μm for all the other wells in the dRT-LAMP device; 40 μm for the thermal expansion wells and 75 μm for all other wells in the multiplexed PCR device). After etching, the SlipChip devices were subjected to the same glass silanization process, where the glass plates were first thoroughly cleaned with piranha mix and dried with 200 proof ethanol and nitrogen gas, and then oxidized in a plasma cleaner for 10 minutes and immediately transferred into a vacuum desiccator for 1.5 hours for silanization with dimethyldichlorosilane. After silanization, the devices were rinsed thoroughly with chloroform, acetone, and ethanol, and dried with nitrogen gas before use. When a glass SlipChip needed to be reused, it was cleaned with Piranha acid first, and then subjected to the same silanization and rinsing procedure described above.

### Example 10

### SlipChip Design with Alignment Markers

The design of the SlipChip device used was the same as in Example 1, with slight modification. The device was modified to include four etched circles that direct the placement of the four red alignment markers. The device contained a total of 1,280 wells (each with a volume of 6 nL) on either half of the chip; however, when the two halves were manipulated to combine the reagents and initiate reactions, only 1,200 individual reactions were initiated.

### Example 11

### Real-Time dRT-LAMP Assay

400 μL plasma containing a modified HIV virus (5 million copies/mL, part of AcroMetrix® HIV-1 Panel Copies/mL) was loaded onto the iPrep™ PureLink® Virus cartridge. The cartridge was placed in the iPrep™ purification instrument and the purification protocol was performed according to the manufacturer's instructions. The elution volume was 50 μL. The purified HIV viral RNA was diluted 10, 10², 10³ fold in 1 mg/mL BSA solution, aliquoted and stored at −80° C. for further use. HIV viral RNA purified from patient plasma was also aliquoted and stored at −80° C. upon receipt.

### Example 12

### Digital LAMP Assay in SlipChip

HIV-1 viral RNA purification protocol from AcroMetrix® HIV-1 Panel Copies/mL was used to generate copies of HIV-1 RNA. The first solution, which was used for amplifying HIV-1 RNA using the two-step dRT-LAMP method, contained the following: 10 μL RM, 1 μL BSA, 0.5 μL EXPRESS SYBR® GreenER™ RT module (part of EXPRESS One-Step SYBR® GreenER™ Universal), 0.5 μL BIP primer (10 μM), various amounts of template, and enough nuclease-free water to bring the volume to 20 μL. The second solution contained 10 μL RM, 1 IA BSA, 2 μL EM (from LoopAmp® RNA amplification kit), 1 μL or 2 μL FD, 2 μL other primer mixture (20 μM FIP, 17.5 μM FIP, 10 μM LooP_B/Loop_F, and 2.5 μM F3), 1 μL Hybridase™ Thermostable RNase H, and enough nuclease-free water (Fisher Scientific) to bring the volume to 20 μL. The first solution was loaded onto a SlipChip device and incubated at 50° C. for 10 min, and then the second solution was loaded onto the same device and mixed with the first solution. The entire filled device was incubated at 60° C. for 60 minutes. The reaction was repeated at 57° C. and 63° C. for 60 minutes.

### Example 13

### Two-Step RT-LAMP Assay

For two-step RT-LAMP amplification, a first solution (20 μL) containing 10 μL RM, 1 μL BSA, 0.5 μL EXPRESS SYBR® GreenER™ RT module, 0.5 μL BIP primer (10 μM), various amounts of template, and nuclease-free water, was first incubated at 50° C. for 10 min and then mixed with a second solution (20 μL), containing 10 μL RM, 1 μL BSA, 2 μL EM, 1 μL or 2 μL FD, 2 μL other primer mixture, 1 μL Hybridase™ Thermostable RNase H, and nuclease-free water. The 40 μL mixture was split into 4 aliquots and loaded onto an Eco real-time PCR machine (Illumina, Inc). For one-step RT-LAMP amplification, a 40 μL RT-LAMP mix contained the following: 20 μL RM, 2 μL BSA (20 mg/mL), 2 μL EM, 2 μL FD, 2 μl of primer mixture, various amount of template solution, and nuclease-free water. The mixture was split into 4 aliquots and loaded onto the Eco real-time PCR machine. Data analysis was performed using Eco software.

### Example 14

### Two-Step dRT-LAMP Amplification on SlipChip

To perform two-step dRT-LAMP amplification on a SlipChip, the first solution (equivalent to the one described above) was loaded onto a SlipChip device and incubated at 50° C. for 10 min. Then a second solution (equivalent to the one described above) was loaded onto the same device and mixed with the first solution. The entire filled device was incubated at 60° C. for 60 min. The reaction was repeated at 57° C. and 63° C. for 60 minutes.

### Example 15

### PCR Amplification on a Multiplexed SlipChip

The PCR mixture used for amplification of Staphylococcus aureus genomic DNA on a multiplexed SlipChip contained the following: 10 μL 2× SsoFast Evagreen SuperMix (BioRad, CA), 1 μL BSA (20 mg/mL; Roche Diagnostics), 1 μl of 1 ng/μL gDNA, 0.5 μL SYBR Green (10×) and 7.5 μL nuclease-free water. Primers were pre-loaded onto the chip using a previously described technique. The PCR amplification was performed with an initial 95° C. step for 5 min, and then followed by 40 cycles of: (i) 1 min at 95° C., (ii) 30 sec at 55° C., and (iii) 45 sec at 72° C. An additional 5 min at 72° C. was performed to allow thorough dsDNA extension. Genomic DNA (Staphylococcus aureus, ATCC number 6538D-5) was purchased from American Type Culture Collection (Manassas, Va.).

### Example 16

### HIV cDNA Synthesis

HIV cDNA was created by reverse transcription of the purified AcroMetrix® HIV RNA using the SuperScript III First-Strand Synthesis SuperMix according to the manufacturer's instructions. Briefly, a mixture of purified HIV RNA (10-fold diluted from the direct elution), 100 nM B3 primer, 1× Annealing buffer, and water were heated to 65° C. for 5 minutes and then placed on ice for 1 minute. A reaction mix and SuperScript III/RNase Out enzyme mix were added to the reaction for a final volume of 40 μl, and the mixture was placed at 50° C. for 50 minutes. The mixture was then heated to 85° C. for 5 minutes to deactivate the reverse transcriptase, chilled on ice, split into 5 μL aliquots, and frozen at −20° C. until further use. Biotin-labeled DNA was created in a PCR reaction containing a 1:50 dilution of the HIV cDNA, 500 nM biotin-B3 and F3 primers, 500 μM dNTPs, 1 U/μL Phusion DNA polymerase and 1× of the associated HF buffer mix. After an initial 1 minute enzyme activation step at 98° C., the reaction was cycled 39 times at 98° C. for 10 s, 58° C. for 15 s, and 72° C. for 15 s, and finished with a 5 minute polishing step at 72° C. The resulting DNA product was run on a 1.2% agarose gel in TBE buffer stained with 0.5 μg/mL ethidium bromide. The specific band was cut out and purified using the Wizard SV gel and PCR cleanup kit according the manufacturer's instructions and eluted into 50 μl of nuclease-free water. 50 μl of streptavidin MyOne T1 magnetic beads were primed by slow-tilt rotation for 24 hours in 20 mM NaOH. The beads were washed 1 time with water and 4 times with binding buffer (5 mM Tris, 0.5 mM EDTA, 1 M NaCl, 0.05% Tween-20) and resuspended in 30 μl of 2× concentrated binding buffer. 30 μl of PCR product was added to the beads and incubated for 15 minutes while gently rotating to allow binding of the DNA to the magnetic beads. The beads were separated with a magnet, the supernatant was removed, and the beads were resuspended in 40 μL of 20 mM NaOH and incubated for 10 minutes on a rotator to separate the non-biotinylated strand. The beads were then separated with a magnet, and the supernatant containing the ssDNA was collected and mixed with 20 μl of 40 mM HCl. The resulting ssDNA was then purified using an ssDNA/RNA cleaner and concentrator kit, eluted in 20 μL water, and run on an Agilent RNA nano bioanalyzer to confirm the size and integrity of the final product.

### Example 17

### Amplification of HIV Viral RNA Using a One-Step RT-LAMP Method

To amplify HIV viral RNA using the one-step RT-LAMP method, the RT-LAMP mix contained the following: 20 μL RM, 2 μL BSA (20 mg/mL), 2 μL EM, 2 μL FD, 2 μl of primer mixture (20 μM BIP/FIP, 10 μM LooP_B/Loop_F and 2.5 μM B3/F3), various amount of template solution, and enough nuclease-free water bring the volume to 40 μL. The solution was loaded onto a SlipChip and heated at 63° C. for 60 minutes.

### Example 18

### Amplification of HIV Viral RNA Using a Two-Step RT-LAMP Method

To amplify HIV viral RNA using the two-step RT-LAMP method, the first solution contained the following: 10 μL RM, 1 μL BSA, 0.5 μL EXPRESS SYBR® GreenER™ RT module (part of EXPRESS One-Step SYBR® GreenER™ Universal), 0.5 μL BIP primer (10 μM), various amount of, and enough nuclease-free water to bring the volume to 20 μL. The second solution contained: 10 μL RM, 1 μL BSA, 2 μL DNA polymerase solution (from LoopAmp® DNA amplification kit), 1 μL or 2 μL FD, 2 μL other primer mixture (20 μM FIP, 17.5 μM FIP, 10 μM LooP_B/Loop_F and 2.5 μM F3), 1 μL Hybridase™ Thermostable RNase H, and enough nuclease-free water to bring the volume to 20 μL. The first solution was loaded onto a SlipChip device and incubated at 37° C. or 50° C., then the second solution was loaded onto the same device and mixed with the first solution, and the entire device was incubated at 63° C. for 60 minutes.

### Example 19

### Amplification of 1-DNA Using a Digital LAMP Method

To amplify λ-DNA, the LAMP mix contained the following: 20 μL RM, 2 μL BSA (20 mg/mL), 2 μL DNA polymerase, 2 μL FD, 2 μl of primer mixture (20 μM BIP/FIP, 10 μM LooP_B/Loop_F and 2.5 μM B3/F3), various amount of template solution, and enough nuclease-free water to bring the volume to 40 μL. The same loading protocol as above was performed and the device was incubated at 63° C. for 70 minutes.

### Example 20

### Amplification of ssDNA Using a Digital LAMP Method

To amplify ssDNA, the LAMP mix contained the following: 20 μL RM, 2 μL BSA, 2 μL DNA polymerase, 2 μL FD, 2 μL of primer mixture (20 μM BIP/FIP, 10 μM LooP_B/Loop_F and 2.5 μM B3/F3), various amount of template solution, and enough nuclease-free water to bring the volume to 40 μL. The same loading protocol as above was performed and the device was incubated at 63° C. for 60 minutes.

### Example 21

### dRT-PCR Amplification of HIV Viral RNA on a SlipChip

To amplify HIV viral RNA, the RT-PCR mix contained the following: 20 μL 2× Evagreen SuperMix, 2 μL BSA, 1 μL EXPRESS SYBR® GreenER™ RT module, 1 μL each primer (10 μM), 2 μL template, and enough nuclease-free water to bring the volume to 40 μL. The amplification was performed at the same conditions as reported before except for a shortened reverse transcription step of 10 minutes.

### Example 22

### Quantification of the HIV Viral RNA Results by Four Different Digital Chemistries

Quantification results of HIV viral RNA by four different digital chemistries were compared—dRT-PCR with two pairs of primers, and one- and two-step dRT-LAMP—to quantify HIV viral RNA at four dilutions using SlipChip devices. HIV viral RNA concentration was calculated based on the number of observed positive wells (“digital counts”) on a single device according to the Poisson analysis method discussed in a previous paper. All experiments were performed in duplicate and negative control experiments with no HIV viral RNA added were performed in parallel; no positive wells were observed in the negative controls.

### Example 23

### Design of a Glass SlipChip for Performing dRT-LAMP

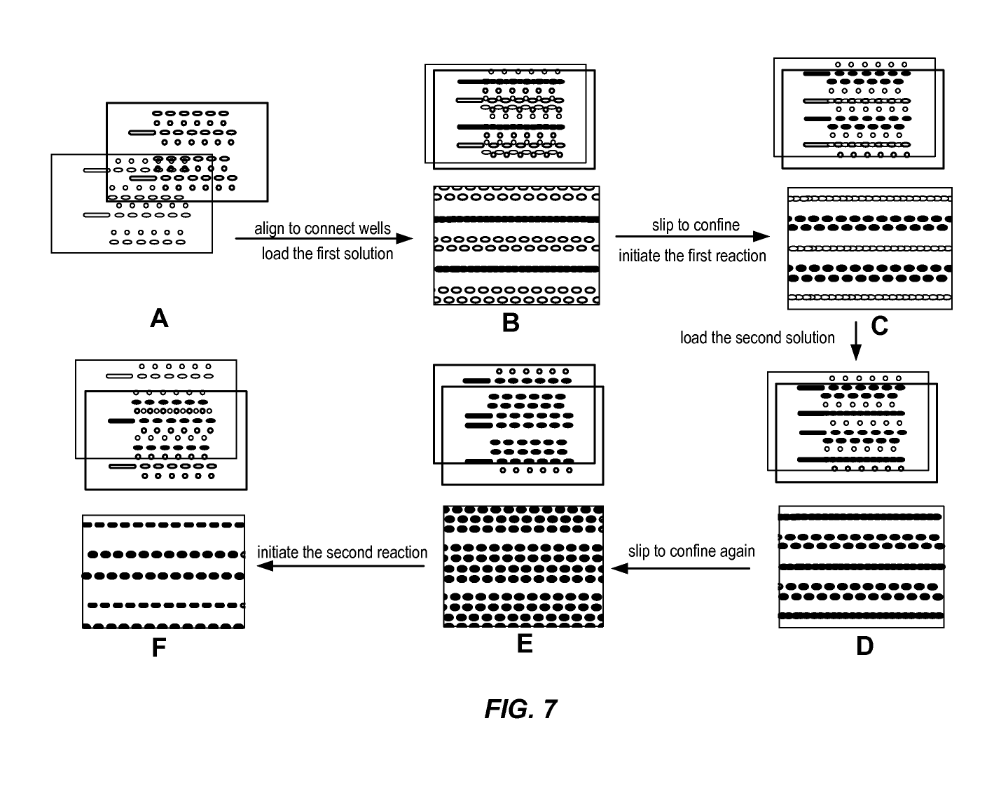

A glass SlipChip device for performing dRT-LAMP was designed in two steps. The device was composed of two glass plates with wells and channels etched on their facing sides (FIG. 7A). The plates of the chip were assembled and aligned to allow for the loading, compartmentalization, incubation, and mixing of reagents in multiple steps. This chip was reminiscent of but not the same as the chip previously described for digital RPA. First, a buffered solution containing template, primer, and RT enzyme was loaded into wells on the chip (FIG. 7B). Next, the plates of the chip were slipped relative to one another to confine single HIV viral RNA molecules into droplets (FIG. 7C). A first incubation step was performed here to allow for reverse transcription. cDNA was synthesized from RNA in each compartment during the reverse transcription step. Then a second solution containing the LAMP reagent mixture and other primers was loaded (FIG. 7D) and split into compartments by slipping (FIG. 7E). Finally, each of the compartments containing a cDNA molecule was combined with a compartment containing LAMP reagents and the entire device was incubated at 63° C. for amplification.

### Example 24

### Compatibility of dRT-LAMP Chemistry with a Plastic SlipChip Device

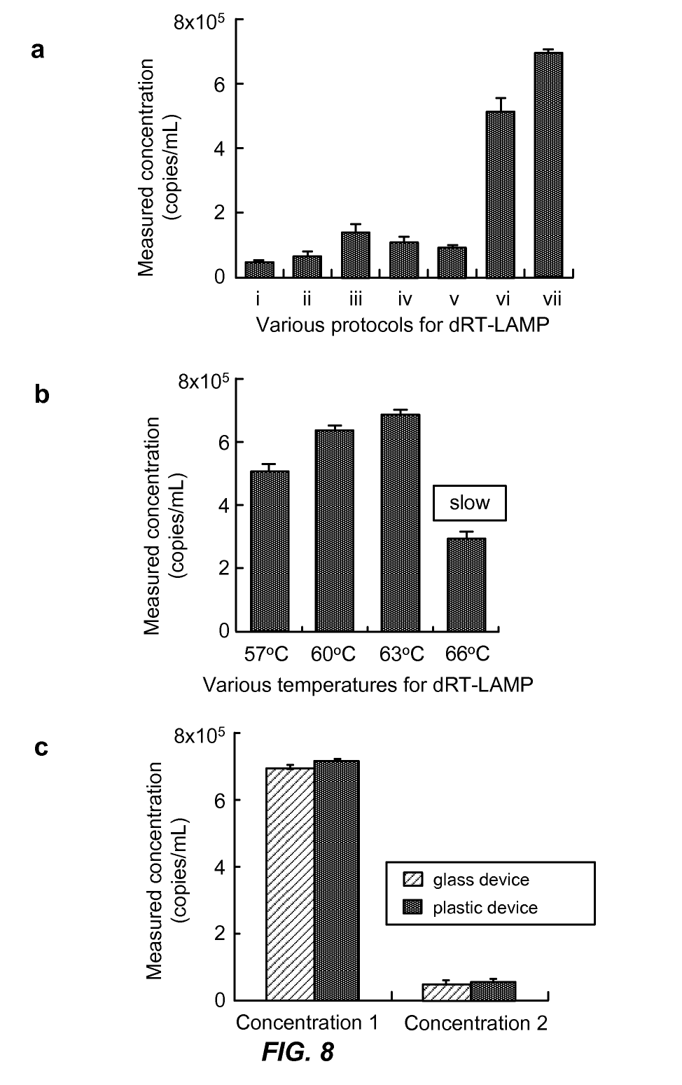

To test the compatibility of this dRT-LAMP chemistry with a plastic SlipChip device, a two-step dRT-LAMP of HIV viral RNA on a plastic SlipChip device with the same design as the glass device was used and the method of Example 18 was used (FIG. 8C).

### Example 25

### Sensitivity of dRT-LAMP in the Presence of Mutation

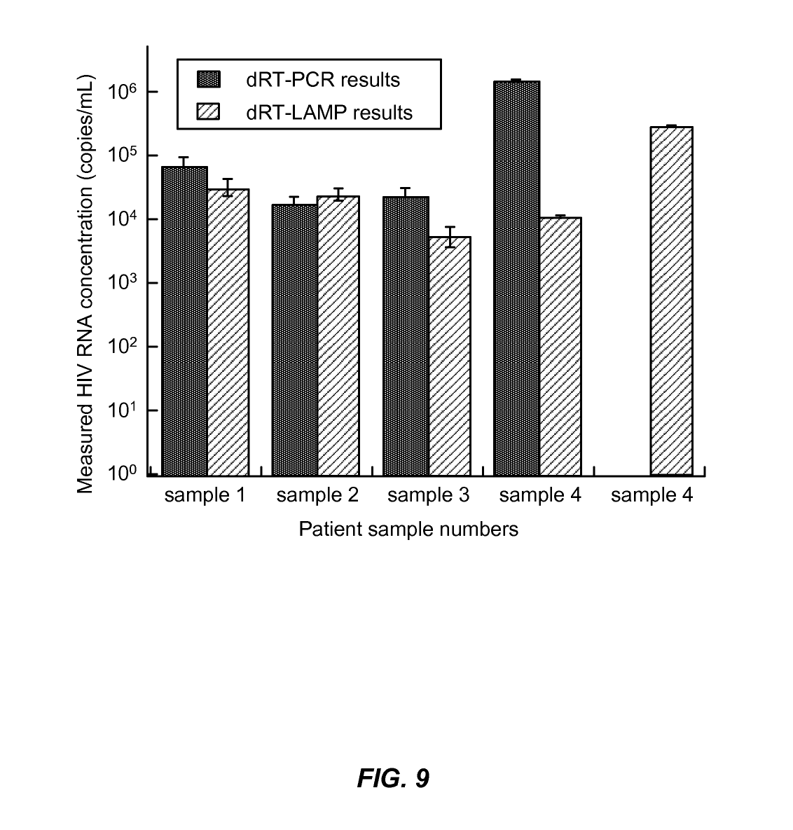

To evaluate the sensitivity of this dRT-LAMP method to the presence of mutations, the performance of two-step dRT-LAMP using HIV viral RNA purified from patient samples was tested and compared these results to measurements from dRT-PCR (FIG. 9). Plasma samples from four different patients were purified using a Roche TNAI kit. Both two-step dRT-LAMP with p24 primers and dRT-PCR with LTR primers were performed to quantify the RNA concentration. The dRT-LAMP quantification results were 46%, 134%, 24%, and 0.74% relative to the corresponding dRT-PCR results, respectively. The p24 region of the purified HIV viral RNA was sequenced. There were 3, 2, 4 and 5 point mutations in the priming regions of samples #1, 2, 3, and 4, respectively.

### Example 26

### Microscope Image Acquisition and Analysis

Fluorescence images were acquired using a Leica DMI 6000 B epi-fluorescence microscope with a 5×/0.15 NA objective and L5 filter at room temperature. The bright-field image and the fluorescence images in real-time dRT-LAMP experiments were acquired using a Leica MZ 12.5 Stereomicroscope. All the images were analyzed using MetaMorph software (Molecular Devices, Sunnyvale, Calif.). Images taken in each experiment were stitched together and a dark noise background value of 110 units was subtracted before the image was thresholded. The number of positive wells was automatically counted using the integrated morphology analysis tool based on intensity and pixel area. The concentrations of HIV-1 RNA were calculated based on Poisson distribution, as described in a previous publication.

Typical fluorescence values for the negative wells were at 80±10. Fluorescence values for the positive wells were centered at 350±100.

### Example 27

### Statistical Analysis of Data Sets Obtained at Different Temperatures

The t-test was used to evaluate whether the means of two different data sets were statistically different. The p value obtained in this process was the probability of obtaining a given result assuming that the null hypothesis was true. A 95% confidence level, which corresponded to p=0.05, or a 5% significance level, was commonly acceptable. It was typically assumed that the concentrations of two samples were different when p<0.05. Here, a p value to evaluate the performance of two-step dRT-LAMP was used in various imaging conditions—with a microscope, with a cell phone and a shoe box, and with a cell phone in dim lighting. When all data for one concentration from different temperatures were pooled and compared them to data acquired at another concentration, the highest p value among the three imaging conditions was 6.7×10⁻⁷. Thus, the two concentrations were clearly distinguishable and the null hypothesis, which stated that both concentrations were equivalent, was rejected. The two closest subsets (2×10⁵ copies/mL at 57° C. and 1×10⁵ copies/mL at 63° C.) were also compared and their p-value under each set of imaging conditions were calculated. The p-values were still below 0.05 for all three conditions.

Additionally, normal distributions were used as visual guides for data interpretation. Normal distributions instead of theoretical t-distributions were used because standard deviations from the data were determined and there was no visible overlap between the data sets corresponding to the two concentrations.

### Example 28

### Imaging of SlipChip dRT-LAMP Device with Cell-Phone Camera

Cell phone imaging of dRT-LAMP devices was performed with the devices tilted at about and or 10 degrees relative to the cell phone plane to prevent direct reflection of the flash into the lens. All images were taken using the standard cell phone camera application. The white balance was set to automatic, the ISO was set at 800, the exposure value was set at +2, the focus mode was set to “close-up,” and the resolution was adjusted to 8 MP.

### Example 29

### Imaging of SlipChip Multiplexed PCR Device with Cell-Phone Camera

Cell phone imaging of multiplexed PCR devices was performed by imaging the devices in a shoebox painted black. The white balance was set to automatic, the ISO was set at 1600, the exposure value was set at +4, the focus mode was set to “close-up,” and the resolution was adjusted to 8 MP. Images were processed using a free Fiji image processing package available on the Internet.

### Example 30

### Measuring Robustness of dRT-LAMP with Respect to Temperature

The robustness of the dRT-LAMP method to temperature variation was tested in the temperature range from 57° C. to 66° C. The first reverse transcription step was performed at 50° C. for 10 minutes in all experiments and the second step was performed at different temperatures. The device was imaged every minute using a stereomicroscope to get a real-time measurement of the digital counts. It was observed that below 63° C., the reactions proceeded quickly enough to yield observable digital counts by 60 minutes, and results were comparable over this temperature range of 57° C. to 63° C. Although the highest reaction rate was observed at 57° C., slightly higher digital counts were obtained at 63° C. At 66° C., the reaction went slower, and at 60 minutes very few positive wells were observed. After 90 minutes of reaction time, the digital counts increased but were still lower than that at 63° C. (FIG. 8B). Further extending the reaction time caused false positives. The similarity of digital counts over the 57° C. to 63° C. range suggests that digital LAMP should give reasonably robust results despite small temperature fluctuations, as was observed for RPA previously.

### Example 31

### Measuring Robustness of Real-Time RT-LAMP with Respect to Temperature

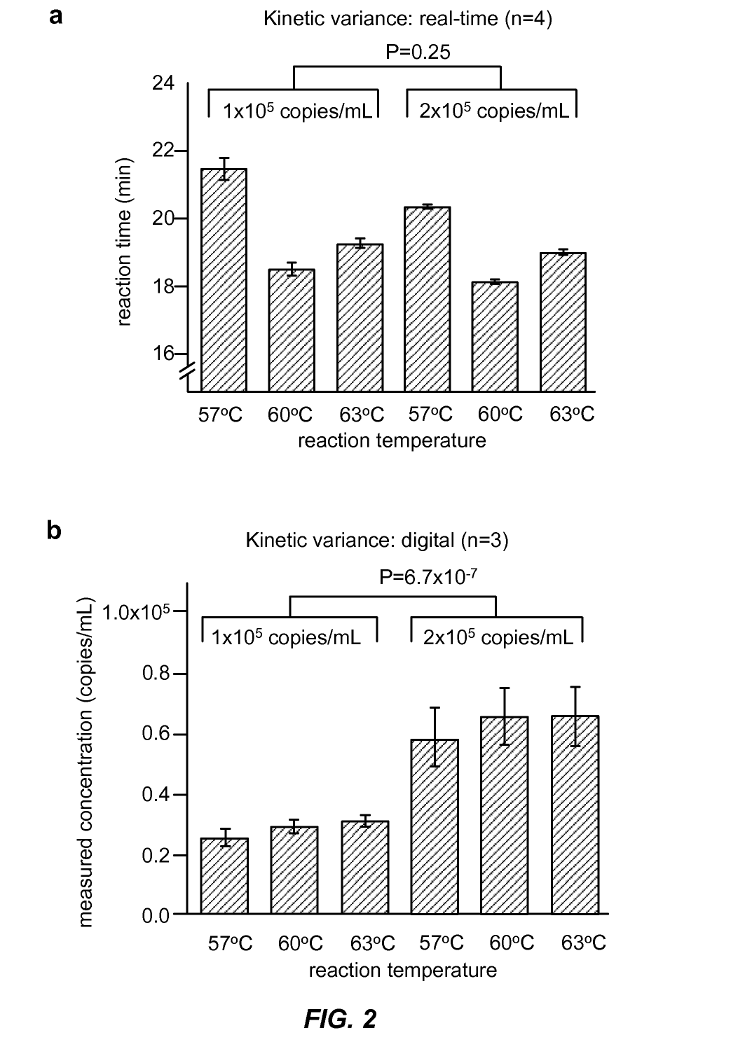

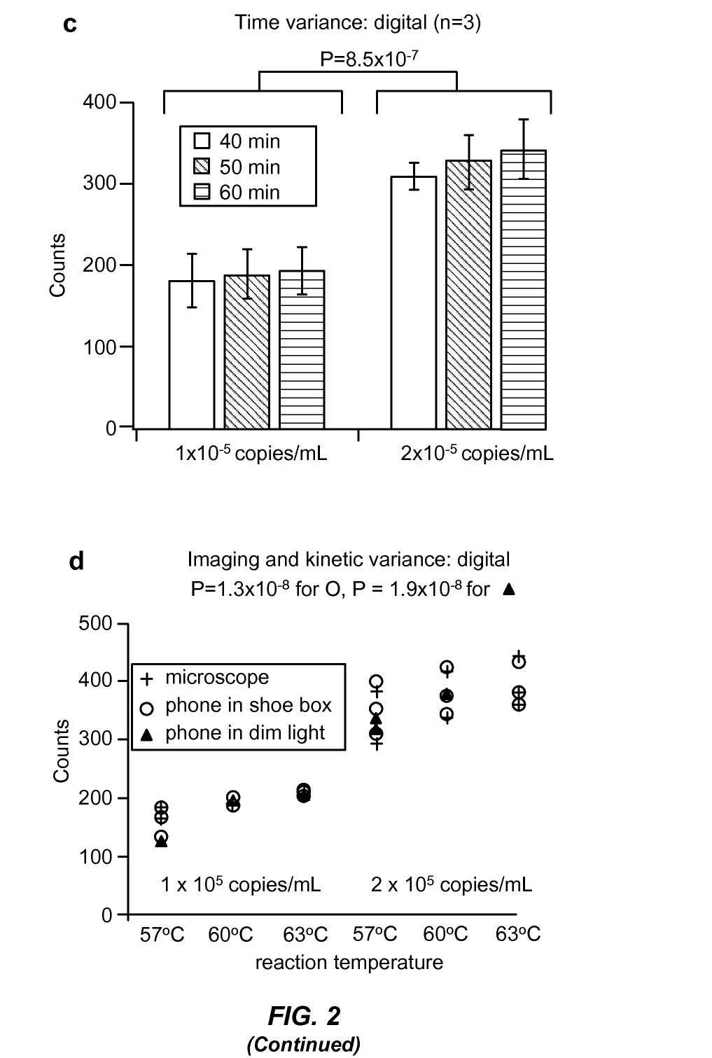

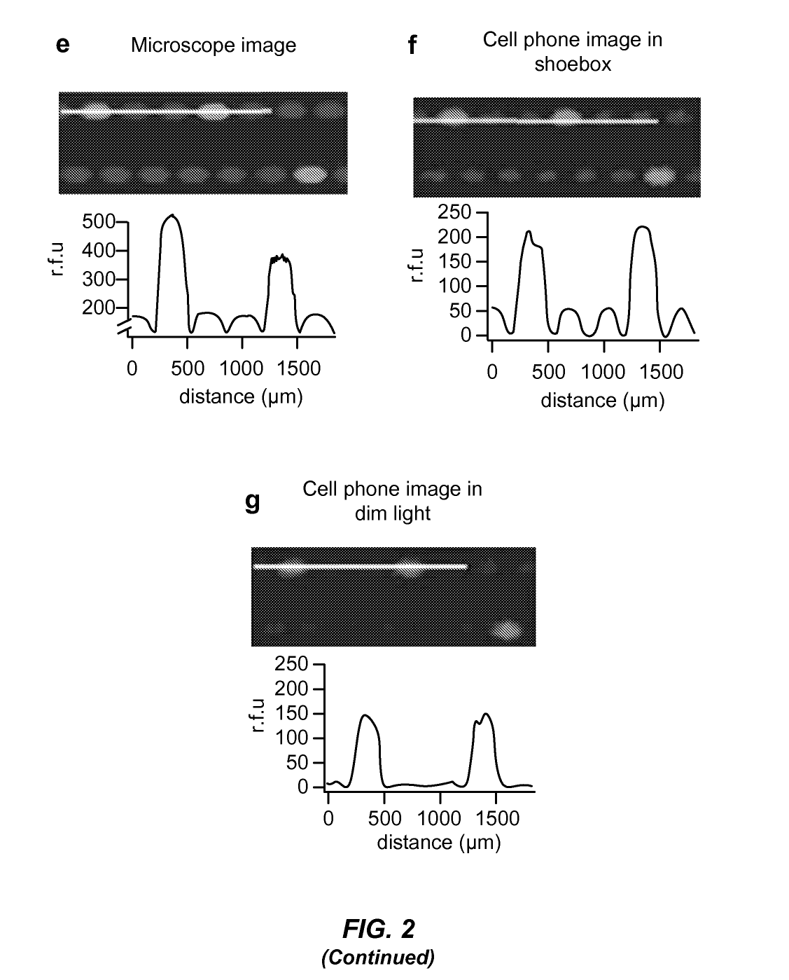

The robustness of the quantitative measurements by real-time RT-LAMP assays were tested with respect to changes in temperature. The robustness of a two-step real-time RT-LAMP assay to temperature fluctuations using a commercial instrument (FIG. 2a) was tested. The precision of the assay for measuring two concentrations (1×10⁵ copies/mL and 2×10⁵ copies/mL) of HIV-1 RNA at three temperatures over a 6-degree temperature range (57° C., 60° C., 63° C.) was tested by comparing the reaction time for these two concentrations measured on an Eco real-time PCR machine. At each individual temperature, the real-time RT-LAMP assay could successfully distinguish between the two concentrations (at 57° C. p=0.007, at 60° C. p=0.01, at 63° C. p=0.04, the null hypothesis being that the two concentrations were identical). The assay, however, was not robust to temperature fluctuations: changes of 3° C. introduced a larger change in the assay readout (reaction time) than the 2-fold change in the input concentration. Therefore, when temperature is not controlled precisely, this real-time RT-LAMP assay cannot resolve a 2-fold change in concentration of the input HIV-1 RNA.

### Example 32

### Comparison of Robustness Between dRT-LAMP and Real-Time RT-LAMP

Robustness of the digital RT-LAMP assay was compared to the real-time RT-LAMP with respect to changes in temperature (FIG. 2b). For the dRT-LAMP experiments, the concentrations of HIV-1 RNA were determined by counting the number of positive wells on each chip after a 60-min reaction and then using Poisson statistics. The dRT-LAMP assay could also distinguish between the two concentrations at each temperature (at 57° C. p=0.03, at 60° C. p=0.02, at 63° C. p=0.02). In contrast to the real-time assay, the dRT-LAMP assay was robust to these temperature changes and resolved a 2-fold change in concentration despite these fluctuations (p=7×10⁻⁷). In these experiments, a Leica DMI-6000 microscope equipped with a Hamamatsu ORCA R-2 cooled CCD camera was used to image the dRT-LAMP devices. This setup provides an even illumination field and, therefore, intensity of the positive well was not a function of position.

### Example 33

### Measuring Robustness with Respect to Reaction Time

The robustness of the dRT-LAMP assay was tested with respect to variance in reaction time. dRT-LAMP reactions were performed with concentrations of 1×10⁵ and 2×10⁵ copies/mL at a reaction temperature of 63° C. and imaged the reaction every minute using a Leica MZFLIII fluorescent stereomicroscope. At each time point, the number of positive reactions was counted, and the results were averaged over three replicates (FIG. 2c). For each of the two concentrations, the raw counts at 40-, 50-, and 60 min-reaction times were grouped together. Statistical analysis was used to reject the null hypothesis that these groups were the same (p-value of 8.5×10⁻⁷).

### Example 34

### Measuring Robustness with Respect to Imaging Conditions

The robustness of the dRT-LAMP assay to poor imaging conditions was tested using a Nokia 808 PureView cell phone with simple optical attachments. The flash function of the cell phone was used to excite fluorescence through an excitation filter attached to the phone, and the camera of the cell phone was used to image fluorescence through an emission filter also attached to the cell phone. The results obtained with the cell phone were compared with those obtained with a microscope (FIG. 2d). The cell phone's imaging abilities were tested under two lighting conditions: first, the dRT-LAMP assays were photographed in a shoe box, and second, in a dimly lit room with a single fluorescent task light in a corner. The light intensity at the point where the measurements were taken in the dimly lit room was ˜3 lux as measured by an AEMC Instruments Model 810 light meter.

To evaluate whether imaging with a cell phone yields robust results, statistical analysis of was performed on data obtained by cell phone imaging under each of the two lighting conditions. For imaging with a shoe box, all data obtained at the first concentration (1×10⁵ copies/mL) across all three temperatures were grouped into a first set, and all data obtained at the second concentration (2×10⁵ copies/mL) across all three temperatures were grouped into a second set. Next, a p-value of 1.3×10⁻⁸ for the two sets (the null hypothesis being that the two concentrations were identical) were calculated, suggesting that this imaging method could be used to differentiate between the two concentrations both at constant temperatures and even despite temperature changes. When this procedure for imaging in a dimly lit room was repeated, a p-value of 1.9×10⁻⁸ was calculated, indicating that the two concentrations could be distinguished with statistical significance in this scenario as well. Therefore, this dRT-LAMP assay was robust to the double perturbation of non-ideal imaging conditions and temperature fluctuations.

### Example 35

### Digital PCR (dPCR) Assay and Comparison to LAMP Assay

Whether other digital assays, such as digital PCR (dPCR), were sufficiently robust to poor imaging conditions to be analyzed with a cell phone. PCR amplification monitored with an intercalating dye such as Evagreen produces only a 2- to 4-fold change in fluorescence intensity as the reaction transitions from negative to positive. The absolute intensity of fluorescence in the positive reaction in dPCR was approximately 15 times lower than that in dRT-LAMP monitored with the calcein dye. When a dPCR experiment using the same reaction volumes as those in the dRT-LAMP assays were conducted, the inventors could easily distinguish positive from negative counts when the chip was imaged using a microscope, as expected, but no fluorescent signal could be observed with the cell-phone method. To confirm that this limitation was due to lack of fluorescence intensity, the cell phone's ability to image the results of a spatially multiplexed PCR chip was tested. This chip uses larger reaction volumes (78 nL as opposed to 6 nL), thus enabling more fluorescent light to be emitted and collected per well. In this chip (FIGS. 3a, b and 4), multiple primer pairs are preloaded into one set of wells, a sample is loaded into the second set of wells, and a “slip” combines the two sets of wells, thus enabling subsequent PCR amplification. Here, a five-plexed assay was used, in which one primer set was specific to the S. aureus genome (FIG. 3b). When S. aureus genomic DNA was loaded onto the device and the PCR reaction was performed, no non-specific amplification was observed and a positive result was indicated by the appearance of the pattern on the device, as designed. This pattern, formed by PCR amplification in these larger wells, could be visualized by the cell phone (FIG. 3c). These experiments indicated that the robustness of dRT-LAMP amplification to cell-phone imaging was not due to the particular characteristics of the cell phone, but rather due to the bright readout signal provide by LAMP.

### Example 36

### Robustness of dRT-LAMP Imaging with Respect to Automated Processing and Analysis

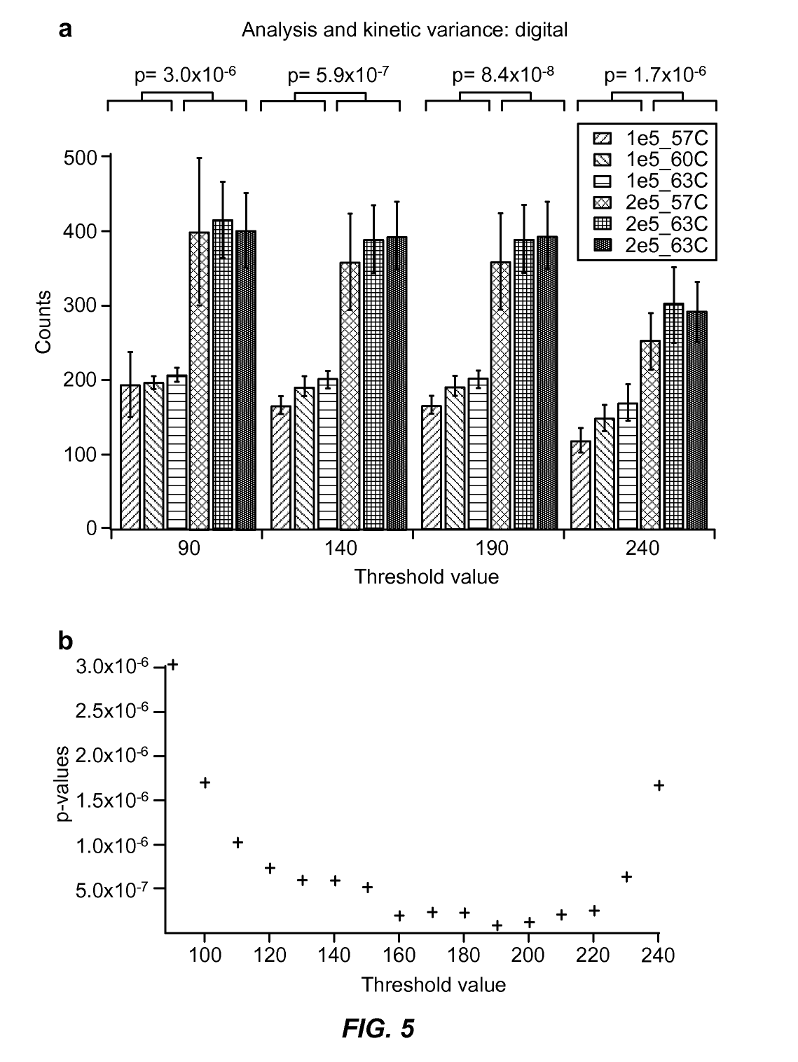

The combination of dRT-LAMP amplification chemistry and cell phone imaging was tested for robustness to automated processing of images and data analysis. When high-quality images, such as those taken with a microscope, are available, image processing and quantification of the positive signals can be performed simply by setting an intensity threshold and then counting the number of spots on the resulting image that exceed this threshold. For example, a threshold of 190 a.u. was set for the data obtained with the microscope, and similar results were obtained by adjusting that threshold by as much as 150 units (FIG. 5).

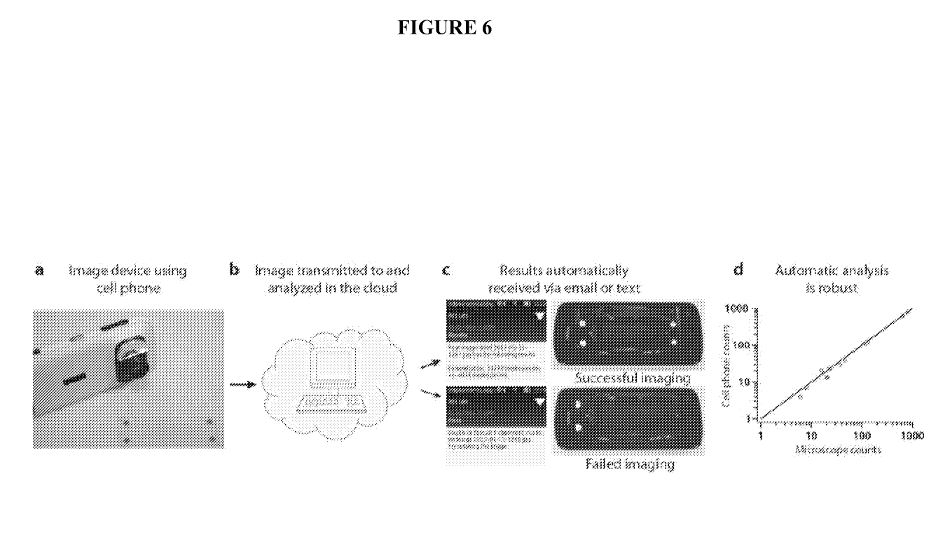

However, images taken with a cell phone were initially unsuitable for two reasons: (i) the short focal length (6 cm) creates significant variation in the illumination intensity of the flash, and (ii) the imaging sensor has a much lower signal-to-noise ratio than those typically found in scientific instrumentation. To overcome these challenges, a custom image processing algorithm was written and implemented it in Labview software. Once an image was taken, it was automatically transferred to a remote server in “the cloud” (FIG. 6b). The uploaded file was automatically analyzed by the server, and then the results were reported via email. The inventors included error detection in the custom algorithm to ensure that the image included the device in its entirety (FIG. 6c). This detection algorithm looked for four red circles on the device (FIG. 6a), and if fewer than four were found, it generated an error message (FIG. 6c, lower panel). The robustness was tested of this cell phone imaging procedure to automated processing by directly comparing microscope images results quantified with Metamorph to cell phone images quantified with Labview over more than a hundred-fold concentration range (FIG. 6d). A line of best fit of the compared data was found to have a slope of 0.968 and an R² value of 0.9997, suggesting that this digital assay is robust to automated image processing even under poor imaging conditions.

### Example 37

### Barcode Used in SlipChip Imaging and Analysis

A QR 2 dimensional barcode is designed that contains the following information: patient name, unique ID number, date of assay, type of SlipChip used, spacing of array of small reaction vessels (or analysis regions) on the SlipChip. The barcode is printed to an adhesive label and affixed to a SlipChip. A small sample is taken from the patient, and injected into the SlipChip. An assay such as DNA amplification is run in the SlipChip. A cell phone is used to take capture an image of the SlipChip and the affixed barcode. The raw image is synced through the cloud to another device. The image of the barcode is processed by software on the computer and the encoded information is saved to a database. Additional information on how to process the rest of the image is extracted from the encoded data, then used to instruct the software on how to proceed with image analysis. The image is analyzed using the methods described herein and the information decoded from the barcode to determine the conclusion of the assay. The conclusion description is stored in a database to be displayed, transmitted, or downloaded as desired.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for generating sample data comprising:
i) emitting a set of photons from a light source in a short burst, the burst lasting from about 5/1,000,000 of a second to about one second, wherein at least a portion of the photons contact the sample;
ii) collecting at least one photon with an image sensor to create sample data, wherein the collected photon had contacted the sample;
iii) processing the sample data to create a binary quantification of nucleic acids in the sample;
iv) analyzing the binary quantification of nucleic acids to generate a conclusion description relating to the sample.

2. The method according to claim 1, wherein the quantification of nucleic acids in the sample is used to detect a non-nucleic acid component of the sample.

3. The method according to claim 2, wherein the non-nucleic acid component is selected from the group comprising cells, proteins and viruses.

4. The method according to claim 1, wherein the collected photon was one of the photons emitted from the light source in a short burst.

5. The method according to claim 1, wherein the photons comprise photons in the visible spectrum.

6. The method according to claim 1, wherein the photons comprise photons in the UV spectrum.

7. The method according to claim 1, wherein the light source is a camera flash or flash bulb.

8. The method according to claim 1, wherein the light source is a Xenon flash.

9. The method according to claim 1, wherein the light source is a light emitting diode (LED).

10. The method according to claim 1, wherein the image sensor is a CMOS.

11. The method according to claim 1, wherein the image sensor is a CCD.

12. The method according to claim 1, wherein the intensity of the set of photons emitted is not constant during the length of time of the short burst.

13. The method according to claim 1, wherein the data associated with the sample is an image or set of images that capture(s) a change in optical properties of the sample relative to a previous time point or a standard sample.

14. The method according to claim 1, wherein the data associated with the sample is an image or set of images that capture(s) the presence or absence of fluorescence data.

15. The method according to claim 14, wherein the fluorescence data is the result of photons emitted from a fluorescent dye.

16. The method according to claim 15, wherein the fluorescent dye is SYTO9.

17. The method according to claim 15, wherein the fluorescent dye is calcein.

18. The method according to claim 1, wherein the data associated with the sample is an image or set of images that capture(s) the presence or absence of colorimetric data.

19. The method according to claim 1, wherein the data associated with the sample is an image or set of images that capture(s) the presence or absence of translucence data.

20. The method according to claim 1, wherein the data associated with the sample is an image or set of images that capture(s) the presence or absence of translucence versus color data.

21. The method according to claim 1, wherein the data associated with the sample is an image or set of images that capture(s) the presence or absence of opacity data.

22. The method according to claim 1, wherein the data associated with the sample is single image captured completely simultaneously.

23. The method according to claim 1, wherein the data associated with the sample comprises measurements from greater than one spatially-isolated compartment each of the compartments comprising a portion of the sample.

24. The method according to claim 1, wherein processing the data further comprises utilizing size discrimination, shape discrimination, comparison to a standard or set of standards, or comparison by color within a single image to create a digital quantification of nucleic acids in the sample.

25. The method according to claim 1, wherein processing the data further comprises:
i) examining the data associated with sample and measuring for each at least one of the following characteristic thresholds a-e:
a) at least one alignment feature is present and/or in the correct orientation;
b) the data associated with the sample comprises an image in focus;
c) the data associated with the image ensure proper usage of assay;
d) the image comprises a graphical depiction of the intended sample;
e) the dimensions of the sample match the intended dimensions; and
f) the sample was distributed in a single container over a series of containers as intended; and
ii) if one or more of the characteristic thresholds was not met, then adjusting the parameters required to exceed all characteristic thresholds and repeating all steps of claim 1 until an unmet characteristic thresholds is met.

26. The method according to claim 1, wherein the data processing is done with a local computer.

27. The method according to claim 1, wherein the data processing is done by transferring the data to a different device to be processed.

28. The method according to claim 1, wherein at least one of the emitted photons that contacted the sample is of a shifted wavelength due to fluorescence.

29. The method according to claim 1, wherein conclusion description is a description of disease.

30. The method according to claim 29, wherein the conclusion description describes the presence or absence of genetic disorder.

31. The method according to claim 29, wherein the conclusion description is a quantification of a viral load.

32. The method according to claim 29, wherein the conclusion description is a diagnosis of a presence or absence of a viral infection.

33. The method according to claim 29, wherein the conclusion description is a quantification of at least one species of bacterium.

34. The method according to claim 29, wherein the conclusion description is a diagnosis of a presence or absence of a bacterial infection.

35. The method according to claim 1, wherein conclusion description is the quantification of a gene in the sample.

36. The method according to claim 1, wherein conclusion description is determining the presence or absence of a gene or nucleic acid sequence in the sample.

37. The method according to claim 36, wherein conclusion description is determining the presence or absence of a gene in the sample.

38. The method according to claim 39, wherein conclusion description is determining the presence or absence of a DNA or RNA sequence in the sample.

39. The method according to claim 1, wherein conclusion description is determining the presence or absence of a mutation in a gene or a mutation in a nucleic acid sequence in the sample.

40. The method according to claim 1, wherein conclusion description is the quantification of a mutation in a gene or nucleic acid sequence in the sample.

41. The method according to claim 37-40, wherein the gene or nucleic acid sequence is plant derived.

42. The method according to claim 37-40, wherein the gene or nucleic acid sequence is human derived.

43. The method according to claim 37-40, wherein the gene or nucleic acid sequence is virus derived.

44. The method according to claim 37-40, wherein the gene or nucleic acid sequence is bacterium derived.

45. The method according to claim 1, further comprising displaying and/or associating in non-transitory computer readable media database the conclusion description and other information.

46. The method according to claim 45, wherein the other information is information about an organism from which the sample was collected.

47. The method according to claim 46, patient name, age, weight, height, time of sample collection, type of sample, GPS location data pertaining to sample collection and/or data collection, or medical records.

48. The method according to claim 1, further comprising displaying the conclusion description.

49. The method according to claim 48, wherein the conclusion description is displayed to the user.

50. The method according to claim 48, wherein the conclusion description is sent to a different device.

51. The method according to claim 1, wherein the sample comprises at least one nucleic acid.

52. The method according to claim 51, wherein the nucleic acid is obtained from a human.

53. The method according to claim 51, wherein the nucleic acid is obtained from a plant or plant seed.

54. The method according to claim 51, wherein the nucleic acid is obtained from an animal.

55. The method according to claim 51, wherein the nucleic acid is obtained from a bacterium.

56. The method according to claim 51, wherein the nucleic acid is obtained from a virus.

57. The method according to claim 51, wherein the nucleic acid is synthetic.

58. The method according to claim 51, wherein the nucleic acid is derived from an unknown source.

59. The method according to claim 1, wherein the sample further comprises a machine-readable label such as a barcode.

60. The method according to claim 59, the label comprising encoded information relating to the sample shape, sample size, sample type, sample orientation, organism from which the sample was obtained, number of samples in proximity to the label, or instructions for further data analysis.

61. The method according to claim 1, wherein the sample undergoes a nucleic acid amplification reaction prior to contacting the photons.

62. The method according to claim 61, wherein the nucleic acid amplification reaction is a loop mediated amplification (LAMP) reaction.

63. The method according to claim 61, wherein the nucleic acid amplification reaction is a PCR reaction.

64. The method of claim 62, wherein the method is performed at about or at a temperature range of 55-65° C.

65. The method of claim 61-64, wherein at least a portion of the sample is partitioned into an array comprising at least 2 or more containers, wherein the image comprises optical data from the location of each container.

66. The method of claim 65, wherein the optical data is a fluorescent signal or a lack of a fluorescent signal.

67. The method of claim 65, wherein the array is a SlipChip.

68. The method according to claim 61, wherein the nucleic acid that is amplified is RNA.

69. The method according to claim 61, wherein the analysis of the digital quantification of nucleic acids within a sample yields a consistent conclusion description for the sample for at least one of the reaction parameters selected from the group consisting of:
i) reaction occurs in a temperature range between 57° C. and 63° C.;
ii) reaction time between 15 min and 1.5 hours;
iii) humidity is between 0% and 100%; and
iv) background light is between 0 and 6 lux.

70. The method according to claim 69, wherein the consistent conclusion description for the sample for at least two of the reaction parameters.

71. The method according to claim 69, wherein the consistent conclusion description for the sample for at least three of the reaction parameters.

72. The method according to claim 69, wherein the consistent conclusion description for the sample for four of the reaction parameters.

73. The method according to claim 61, wherein the image sensor is part of a cell phone or tablet computer.

74. The method according to claim 1, further comprising at least one of the following steps:
a) detection of a fluorescent region using a cell phone;
b) detection of a fluorescent region using a mobile handheld device;
c) detection of a fluorescent region corresponding to an amplification product from a single molecule;
d) exciting fluorescence using a compact flash integrated with a mobile communication device;
e) transmitting an image and/or a processed image and/or resulting data to a centralized computer;
f) background correction of an image using a combination of color channels;
g) enhancement of fluorescent regions by using one or more filtering algorithms;
h) shape detection using one or more shapes to determine image fidelity;
i) shape detection using one or more shapes to determine the region to be analyzed;
j) shape detection using one or more algorithms to determine positive regions;
k) processing and/or analyzing images and/or data on the centralized computer;
l) optionally archiving the images and/or data;
m) transmitting information back to the mobile device;
n) transmitting an image and/or a processed image and/or resulting data the user;
o) transmitting an image and/or a processed image and/or resulting data to a third party;
p) applying Poisson statistical analysis to quantify the number of fluorescent and non-fluorescent regions; and
q) applying Poisson statistical analysis to quantify concentration based on the number of fluorescent and non-fluorescent regions.

75. The method of claim 1, wherein the light source has a light intensity of at least greater or equal to 100,000 lux.

76. The portable digital device of claim 1, wherein the light is emitted from a mobile phone containing a built-in camera or is a tablet containing a built-in camera.

77. The method of claim 1, wherein the light it filtered.

78. The method of claim 77, wherein the filter comprises a set of filters.

79. The method of claim 78, wherein the set of filters comprises at least one, two, three, four filters or any combination thereof.

80. The method of claim 77, wherein the filters comprises a fluorescent filter.

81. The method of claim 80, wherein the fluorescent filter comprises a dichroic filter and/or a long-pass filter.

82. The method of claim 81, wherein the dichroic filter can be greater than 85% transmission about or at 390-480 nm and less than 1% about or at 540-750 nm.

83. The method of claim 81, wherein the long-pass filter can have blocking of greater than 5 OD and transmission of greater than 90% at wavelengths about or at 530-750 nm.

84. The method of claim 1, wherein the analysis process can take less than one minute.

85. The method of claim 1, wherein the analysis process performs a background correction of an image using a data collected from a second color channel.

86. The method of claim 85, wherein the software algorithm can apply Poisson statistical analysis to quantify the number of fluorescent and non-fluorescent regions.

87. The method of claim 1, wherein the data analysis takes place locally, through a cloud-based service, through a centralized computer located remotely or any combination thereof.

88. The method of claim 1, wherein the method is providing an application for detecting nucleic acids.

89. The method of claim 1, wherein the portable digital device is tilted at an angled position when taking a picture

90. A device for generating sample data, the device comprising:
i) a light source that emits a set of photons in a short burst, the burst lasting from about 5/1,000,000 seconds to about one second, wherein at least a portion of the photons contact the sample;
ii) an image sensor not in alignment with the light source that collects at least a portion of the photons that contacted the sample to create data associated with the sample;
iii) a processor configured to process the sample data to create a binary quantification of nucleic acids in the sample or a wireless connection to transmit the sample data to a different device configured to create a binary quantification of nucleic acids in the sample; and
iv) a processor configured to analyze the binary quantification of nucleic acids to generate a conclusion description relating to the sample.

91. The device of claim 90, further comprising a filter.

92. The device of claim 91, wherein the set of filters comprises at least one, two, three, four filters or any combination thereof.

93. The device of claim 92, wherein the filters comprises a fluorescent filter.

94. The device of claim 93, wherein the fluorescent filter comprises a dichroic filter and/or a long-pass filter.

95. The device of claim 94, wherein the dichroic filter can be greater than 85% transmission about or at 390-480 nm and less than 1% about or at 540-750 nm.

96. The device of claim 95, wherein the long-pass filter can have blocking of greater than 5 OD and transmission of greater than 90% at wavelengths about or at 530-750 nm

97. The device of claim 90, further comprising a screen to display the conclusion description.

98. The device of claim 90, wherein the light source is a camera flash.

99. The device of claim 90, wherein the image sensor is CCD or CMOS.

100. A kit comprising a container comprising:
i) a plurality of small containers;
ii) components of a nucleic acid amplification reaction;
iii) and instructions for use.

101. The kit of claim 100, wherein the plurality of small containers is a SlipChip.

102. The kit of claim 100, further comprising a machine-readable label such as a barcode.

103. The kit of claim 102, the label comprising encoded information relating to the sample shape, sample size, sample type, sample orientation, organism from which the sample was obtained, number of samples in proximity to the label, or instructions for further data analysis.

104. The kit of claim 100, wherein the components of a nucleic acid amplification reaction are located within at least one of the small containers.

105. The kit of claim 100-104, further comprising the device of claim 90.
